SEQUENCE LISTING (46 sequences)

SEQ ID NO: 1 - DNA, 1586 bp, synthetic construct
  misc_feature 1..1586: note = Description of Artificial Sequence: Synthetic polynucleotide
  source 1..1586: mol_type = other DNA
gaaataattttgtttaactttaagaaggagatatacatatgagcgaacctgttccggcagcagcacgtcgtaccattccgggtagcgaacgtccgcctgttgataccgcagcagcagcccgtcaggcagttcctgcagatacccgtgttgaagcaaccgttgttctgcgtcgtcgtgcagaactgccggatggtccgggtctgctgacaccggcagaactggcagaacgtcatggtgcagatccggcagatgttgaactggttacccgtacactgaccggtctgggtgttgaagttaccgcagttgatgcagcaagccgtcgtctgcgtgttgccggtccggcaggcgttctggcagaagcatttggcaccagcctggcacaggttagcacaccggatccgagcggtgcccaggttacccatcgttatcgtgccggtgcactgagcgttccagccgaactggatggtgttgtgaccgcagttctgggtttagatgatcgtccgcaggcacgtgcgcgttttcgtgttgcaacggcagccgcagcaagcgcaggttataccccgattgaactgggtcgtgtttatagctttccggaaggtagtgatggtagcggtcagaccattgcaattattgaattaggtggtggttttgcacagagtgaactggatacctattttgcaggtctgggtattagcggtccgaccgttacagcagttggtgttgatggtggtagcaatgttgcaggtcgtgatccgcagggtgcagatggtgaagttctgctggatattgaagttgcgggtgcactggcaccgggtgccgatgttgttgtttattttgcaccgaataccgatgcaggttttctggatgcagttgcacaggcagcacatgcaaccccgactccggcagccattagcattagctggggtggtagcgaagatacctggacaggtcaggcacgtaccgcctttgatgcggcactggcagatgcagccgcactgggtgttaccaccaccgttgcagccggtgatgatggtagtaccgatcgtgcaaccgatggtaaaagccatgttgattttccggcaagcagtccgcatgcactggcctgtggtggcacccatctggatgccaatgcaaccaccggtgcagttaccagcgaagttgtttggaataatggtgcaggtaaaggtgcaaccggtggcggtgttagcaccgtttttgcccagccgagctggcaggcaagtgccggtgttccggatggccctggtggtaaacctggtcgtggtgtgccggatgttagcgcagttgccgatccgcagaccggttatcgtattcgtgtggatggtcaggatctggttattggtggtacaagcgcagtggcaccgctgtgggcagcactggttgcacgtctggttcaggcaggtcgcgcaaaactgggcctgctgcagccgaaactgtatgcagcaccgaccgcatttcgtgatattaccgaaggtgataatggcgcatatcgtgcaggtcctggttgggatgcatgtacaggcctgggcgttccggttggcaccgcactggcgagcgcactgagttga

SEQ ID NO: 2 - protein, 515 aa, Pseudonocardia sp.
MSEPVPAAARRTIPGSERPPVDTAAAARQAVPADTRVEATVVLRRRAELPDGPGLLTPAELAERHGADPADVELVTRTLTGLGVEVTAVDAASRRLRVAGPAGVLAEAFGTSLAQVSTPDPSGAQVTHRYRAGALSVPAELDGVVTAVLGLDDRPQARARFRVATAAAASAGYTPIELGRVYSFPEGSDGSGQTIAIIELGGGFAQSELDTYFAGLGISGPTVTAVGVDGGSNVAGRDPQGADGEVLLDIEVAGALAPGADVVVYFAPNTDAGFLDAVAQAAHATPTPAAISISWGGSEDTWTGQARTAFDAALADAAALGVTTTVAAGDDGSTDRATDGKSHVDFPASSPHALACGGTHLDANATTGAVTSEVVWNNGAGKGATGGGVSTVFAQPSWQASAGVPDGPGGKPGRGVPDVSAVADPQTGYRIRVDGQDLVIGGTSAVAPLWAALVARLVQAGRAKLGLLQPKLYAAPTAFRDITEGDNGAYRAGPGWDACTGLGVPVGTALASALS

SEQ ID NO: 3 - DNA, 1634 bp, synthetic construct
  misc_feature 1..1634: note = Description of Artificial Sequence: Synthetic polynucleotide
  source 1..1634: mol_type = other DNA
gaaataattttgtttaactttaagaaggagatatacatatggccgatgatagcagcccgaccaccgcagcagatcgtccgacactgcctggtagcgcacgtcgtccggttgcagcagcacaggcagcaggtccgctggatgatgcagcaccgctggaagttaccctggttctgcgtcgtcgtaccgcactgccagcaggcacaggtcgtccggcaccgatgggtcgtgcagaatttgcagaaacccatggtgcagatccggcagatgccgaaaccgttaccgcagcactgaccgcagaaggtctgcgtattaccgcagttgatctgccgagccgtcgtgttcaggttgccggtgatgttgcaacctttagccgtgtttttggtgttagcctgagccgtgttgaaagccctgatccggttgccgatcgtctggttccgcatcgtcagcgtagcggtgatctggcagttcctgctccgctggcaggcgttgtgaccgcagttctgggtttagatgatcgtccgcaggcacgtgcactgtttcgtcctgcagcagccgttgataccacctttactccgctggaactgggtcgtgtttatcgttttccgagcggtacagatggtcgtggtcagcgtctggcaattctggaattaggtggtggttatacccaggcagatctggatgcatattggaccaccattggtctggcagatccgcctaccgttacagcagttggtgttgatggtgcagcaaatgcaccggaaggtgatccgaatggtgccgatggtgaagttctgctggatattgaagttgcgggtgcactggcaccgggtgccgatctggttgtttattttgcaccgaataccgatcgtggttttctggatgccctgagcaccgcagtgcatgccgatccgacaccgaccgcagtgagcattagctggggtcagaatgaagatgaatggaccgcacaggcacgtaccgcaatggatgaagcactggcagatgcagccgcactgggtgttaccgtttgtgcagcagcgggtgatgatggtagcacagataacgcaccggatggtcaggcacatgttgattttccggcaagcagtccgcatgcgctggcatgtggtggtacaaccctgcgtgcggatccggataccggtgaagttagcagcgaaaccgtgtggtttcatggcaccggtcaaggtggtactggtggtggtgtgagcgcagtttttgcagttccggattggcaggatggtgttcgtgttccgggtgatgcagataccggtcgtcatggtcgcggtgttccggatgttagcgcagatgctgatccgagtaccggttatcaggttcgtgtggatggtacggatgcagtgtttggtggcaccagcgcagttagtccgctgtggtctgcactgacctgtcgtctggccgaagcgctgggacagcgtccgggtctgctgcagccgctgatttatgcaggtctgagcgcaggcgaagttgcagccggttttcgtgatgttaccagcggtagcaatggtgcatacgatgcaggtcctggttgggatccgtgcaccggtctgggtgtgccggatggcgaagcactgctggttcgtctgcgtacagcactgggctga

SEQ ID NO: 4 - protein, 531 aa, Modestobacter sp.
MADDSSPTTAADRPTLPGSARRPVAAAQAAGPLDDAAPLEVTLVLRRRTALPAGTGRPAPMGRAEFAETHGADPADAETVTAALTAEGLRITAVDLPSRRVQVAGDVATFSRVFGVSLSRVESPDPVADRLVPHRQRSGDLAVPAPLAGVVTAVLGLDDRPQARALFRPAAAVDTTFTPLELGRVYRFPSGTDGRGQRLAILELGGGYTQADLDAYWTTIGLADPPTVTAVGVDGAANAPEGDPNGADGEVLLDIEVAGALAPGADLVVYFAPNTDRGFLDALSTAVHADPTPTAVSISWGQNEDEWTAQARTAMDEALADAAALGVTVCAAAGDDGSTDNAPDGQAHVDFPASSPHALACGGTTLRADPDTGEVSSETVWFHGTGQGGTGGGVSAVFAVPDWQDGVRVPGDADTGRHGRGVPDVSADADPSTGYQVRVDGTDAVFGGTSAVSPLWSALTCRLAEALGQRPGLLQPLIYAGLSAGEVAAGFRDVTSGSNGAYDAGPGWDPCTGLGVPDGEALLVRLRTALG

SEQ ID NO: 5 - DNA, 1613 bp, synthetic construct
  misc_feature 1..1613: note = Description of Artificial Sequence: Synthetic polynucleotide
  source 1..1613: mol_type = other DNA
gaaataattttgtttaactttaagaaggagatatacatatggattatcagattctgcgtggtagcgaacgtagtccgctgcctggttgtaccgataccggtaaatttccggcagcacatcgtctgcgtgttctgctggcactgcgtcagccggaactggatgcagcagcagcccgtctgctggatacagccggtgatgaactgcctgcaccgctgagccgtgatgcatttgcaacccgttttgcagcagccgcagatgacctgcgtgcagttgaagcatttgcgacccagcatggtctgagcatggaacagaccctggcacatgccggtgttgcaattctggaaggtagcgttcagcagtttgatcgtgcatttcaggttgatctgcgtgattatcgtaaagatgatctgcgctatcgtggtcgtaccggtgcagttagcattccgaccgcactgcatggtgttgttagcgcagttctgggtttagatgatcgtccgcaggcacataccctgccgcaggcgcaggatgcaccagcaccagctggcgcagcagcaccgattgcacgttatacccctccgcagctggcagaactgtatggttttccggaacatgatggtgcaggtcagtgtattggtattattgcattaggtggtggttatgaacgtgcacaactggcagcatattttaccgaactgggtctgccgatgccgcagattgttgatgtactgctggcaggcgcacgtaatcagcctggtggtcagggtcgtaaagcagatattgaagttcagatggatgttcagattgccggtgcaattgcccctggtgccaaactggttgtttattttgcaccgaataccgataatggctttctggaagcaattgtgagcgcaattcatgatcgtgcccatgcaccggatgttattgcaatttcatggggttttacagaaaccctgtggaccgcacagagccgtgcagcatataatcgtgcactgcaggcagcagcgctgatgggtattaccgtttgtattgcaagcggtgatgatggcgcaagtgatggtcagccaggtctgaatgtttgttttccggcaagcagtccgtttgttctggcatgtggtggcacccgtctgcaggttgatgttcaggcacagcatgaacaggcatggtcaggcaccggtggtggccagagtcgtgtttttgcacgtccgcgttggcagcaggcactgacgctgcatggcacccagcagacagcacagccgctgagcatgcgtggtgttccggatgttgcagcaaatgcagatgcagaaaccggttattatgtgcatattgatggtcgtccggcagttatgggtggcaccagtgcagccgcaccggtttgggcagcactgttagcacgtgtttatggcctgaatggtggtcgtcgtgtgtttctgcctccgcgtctgtatgcagttgcagatgtttgtcgtgatattgtggatggtggtaatggtggttttgttgcaagccctggttgggatgcatgtaccggtctgggtgtgccggatggtggccgtattgccgcagccttaggtgccggtccgggtgcaaaaccggcaattaccccgacaggctga

SEQ ID NO: 6 - protein, 524 aa, Xanthomonas translucens
MDYQILRGSERSPLPGCTDTGKFPAAHRLRVLLALRQPELDAAAARLLDTAGDELPAPLSRDAFATRFAAAADDLRAVEAFATQHGLSMEQTLAHAGVAILEGSVQQFDRAFQVDLRDYRKDDLRYRGRTGAVSIPTALHGVVSAVLGLDDRPQAHTLPQAQDAPAPAGAAAPIARYTPPQLAELYGFPEHDGAGQCIGIIALGGGYERAQLAAYFTELGLPMPQIVDVLLAGARNQPGGQGRKADIEVQMDVQIAGAIAPGAKLVVYFAPNTDNGFLEAIVSAIHDRAHAPDVIAISWGFTETLWTAQSRAAYNRALQAAALMGITVCIASGDDGASDGQPGLNVCFPASSPFVLACGGTRLQVDVQAQHEQAWSGTGGGQSRVFARPRWQQALTLHGTQQTAQPLSMRGVPDVAANADAETGYYVHIDGRPAVMGGTSAAAPVWAALLARVYGLNGGRRVFLPPRLYAVADVCRDIVDGGNGGFVASPGWDACTGLGVPDGGRIAAALGAGPGAKPAITPTG

SEQ ID NO: 7 - DNA, 1691 bp, synthetic construct
  misc_feature 1..1691: note = Description of Artificial Sequence: Synthetic polynucleotide
  source 1..1691: mol_type = other DNA
gaaataattttgtttaactttaagaaggagatatacatatgacccgtcatccggttagcgatagcggtgcaagcaatgaacatccggttccggcaggcgcacagtgtatgggtgcatgtgatccggcagaacattttaatgttgttgttattgttcgtcgtcagagcgaacgtgcatttcgtgaactggttgaacgtattgcaacaggtgcaccgggtgcgcagccgattagccgtgaacagtatgaacagcgttttagcgcagatgcagcagatgttgcacgtgttgaagcatttgcaaaaacccatggtctggttgttgtgaaagcagatcgtgatacccgtcgtgttgttctgagcggcaccgttcagcagtataatgcagcatttggtgttgatctgcagcgttttgaacatcaggttggtaaactgaaacagcattttcgtcagccgaccggtccggttcatctgccggaagatctgcatgaagttattaccgcagttgttggtctggatagccgtgcaaaagttcagccgcattttcgcattgatagccagacaccggcaacaccgcctgaaaaagcaagccagcctggtgatggtgttgttcatgcaccgattcgtgcagcacgtgcagttagccgtagctttacaccgctgcagctggcagaactgtatgattttccgccaggtgatggtaaaggtcagtgtattgcactgattgaaatgggtggtggttatgcacagagcgatctggatgcatattttagtgcactgggtgttacccgtccgcgtgtggaagcagttagcgttgatcaggcaaccaatgcaccgagcggtgatccgaatggtccggatgccgaagttaccctggatgttgaaattgccggtgcactggctccgggtgctctgattgcagtttattttgcaccgaatagcgaagccggttttgttgatgccgttagcgcagcactgcatgatagtcagcgtaaagcagcaattattagcattagctggggtgctccggaaagcatttggagccagcagaccctgggtgcactgaatgatgcactgcagaccgcagtggccctgggtgtgaccgtttgttgtgcaagcggtgatagcggtagctcagatggtgttaccgatggtgcagatcatgtggattttccggcaagcagcccgtatgcattaggttgtggtggcacccagctgaccgcagcaaatggtcgtattacccgtgaaaccgtttggggtagcggtgccaatggtgcaaccggtggtggtgttagcgcaacctttgcagttccggcatggcagaaaggtctgaaagtgagccgtggtagtggtgccgcacgtgccctggcactggcacgtcgtggtgttccggatgttgcagccgatgcagatccggcaaccggttatgaagttcatattggtggtatggataccgttgttggtggtacaagcgcagttgctccgctgtgggcagcactggttgcccgtattaatgcaggtagcggtaaagccgcaggttttatcaatgccaaactgtatgcacgtccgggtgcatttaatgatatcaccagcggtagcaatggtgattatgcagcccgtcctggttgggatgcatgtaccggtctgggtacaccggttggtacacgtgttgcagcggcaattggtagcgcatga

SEQ ID NO: 8 - protein, 550 aa, Paraburkholderia sacchari
MTRHPVSDSGASNEHPVPAGAQCMGACDPAEHFNVVVIVRRQSERAFRELVERIATGAPGAQPISREQYEQRFSADAADVARVEAFAKTHGLVVVKADRDTRRVVLSGTVQQYNAAFGVDLQRFEHQVGKLKQHFRQPTGPVHLPEDLHEVITAVVGLDSRAKVQPHFRIDSQTPATPPEKASQPGDGVVHAPIRAARAVSRSFTPLQLAELYDFPPGDGKGQCIALIEMGGGYAQSDLDAYFSALGVTRPRVEAVSVDQATNAPSGDPNGPDAEVTLDVEIAGALAPGALIAVYFAPNSEAGFVDAVSAALHDSQRKAAIISISWGAPESIWSQQTLGALNDALQTAVALGVTVCCASGDSGSSDGVTDGADHVDFPASSPYALGCGGTQLTAANGRITRETVWGSGANGATGGGVSATFAVPAWQKGLKVSRGSGAARALALARRGVPDVAADADPATGYEVHIGGMDTVVGGTSAVAPLWAALVARINAGSGKAAGFINAKLYARPGAFNDITSGSNGDYAARPGWDACTGLGTPVGTRVAAAIGSA

SEQ ID NO: 9 - DNA, 1637 bp, synthetic construct
  misc_feature 1..1637: note = Description of Artificial Sequence: Synthetic polynucleotide
  source 1..1637: mol_type = other DNA
gaaataattttgtttaactttaagaaggagatatacatatggtgcgtcatccgctgcgtggtagcgaacgtaccattccggaagatgcacgtattctgggtgatgcacatccggcagagcagattcgtgcactggttcagctgcgtcgtccgaatgaagcagaactggatgttcgtctgagcggttttgttcatgcacatgcagcaggcaccccgagtccgacaccgctgacacgtgaagaatgggcagcacagtttggtgcagcaaccgatgatattgatgcagttcgtacctttgcacgtgaacatggtctgcaggttgccgaagttaatgttgcagcagccaccgttatgctggaaggtagcgttgaacagttttgtcgtgcatttgatacccatctgcatcgtgttgcacatggtggtagtgaatatcgtggtcgtagcggtccgctgcgcctgccggaaagcctgcaggatgttgttgttgcagttctgggtttagatagccgtccgcaggcagcaccgcattttcgttttgttccgctgccgaccggtagcgtggaacctggtggtattcgtccggcacgtgcagcaccgaccgcaagctatacaccggtgcagctggcacagctgtatggttttccgcaaggtgatggtgcaggtcagtgtattgcatttgttgaattaggtggtggttatcgcgaagatgatctgcgtgcatattttcaagaggttggtatgccgatgccgaccgttaccgcaattccggttggtcagggtgcaaatcgtccgaccggtgatccgagcggtccggatggtgaagtgatgctggatctggaagttgcgggtgcagccgcaccgggtgcaaccctggcagtgtattttaccgttaataccgatgcaggttttgtgcaggcaattaatgcagcaattcatgataccaaactgcgtccgagcgttgttagcattagctggggtgcaccggaaagcgcatggacaccgcaggcaatgcaggccgttaatgccgcactgcagagcgcagcaaccatgggtgttaccgtttgtgcagccagcggtgatagcggtagcagtgatggtcagccggatcgtgttgatcatgttgattttccggcaagcagcccgtatgcactggcatgtggtggcaccagcgttcgtgcaagcggtaatcgtattgccgaagaaaccgtttggaatgatggtgcccgtggtggtgcaggcggtggtggtgttagcaccgtttttgcactgccgagctggcagcaaggtctggcagcccagcagaccggtggtgattcagttccgctggcacgtcgtggtgttccggatgttagcgcagatgcagatccgctgaccggttatgttgttcgcgttgatggtgaaagcggtgttgttggtggtacatcagctgccgcaccgctgtgggcagccctgattgcccgtattaatgcaattaaaggccgtccggcaggttatctgcatgcacgtctgtatcagaatccgggtgcatttaatgatattaagcagggtaataatggtgcctttgccgcagcacctggttgggatgcatgtaccggtctgggtagcccgaaaggtgatgcaattgccaacctgttttga

SEQ ID NO: 10 - protein, 532 aa, unidentified
  source 1..532: note = Burkholderiaceae bacterium
MVRHPLRGSERTIPEDARILGDAHPAEQIRALVQLRRPNEAELDVRLSGFVHAHAAGTPSPTPLTREEWAAQFGAATDDIDAVRTFAREHGLQVAEVNVAAATVMLEGSVEQFCRAFDTHLHRVAHGGSEYRGRSGPLRLPESLQDVVVAVLGLDSRPQAAPHFRFVPLPTGSVEPGGIRPARAAPTASYTPVQLAQLYGFPQGDGAGQCIAFVELGGGYREDDLRAYFQEVGMPMPTVTAIPVGQGANRPTGDPSGPDGEVMLDLEVAGAAAPGATLAVYFTVNTDAGFVQAINAAIHDTKLRPSVVSISWGAPESAWTPQAMQAVNAALQSAATMGVTVCAASGDSGSSDGQPDRVDHVDFPASSPYALACGGTSVRASGNRIAEETVWNDGARGGAGGGGVSTVFALPSWQQGLAAQQTGGDSVPLARRGVPDVSADADPLTGYVVRVDGESGVVGGTSAAAPLWAALIARINAIKGRPAGYLHARLYQNPGAFNDIKQGNNGAFAAAPGWDACTGLGSPKGDAIANLF

SEQ ID NO: 11 - DNA, 1583 bp, synthetic construct
  misc_feature 1..1583: note = Description of Artificial Sequence: Synthetic polynucleotide
  source 1..1583: mol_type = other DNA
gaaataattttgtttaactttaagaaggagatatacatatgccgacctttctgctgcctggtagcgaacagacctgtccgcctggtgcacgttgtgttggtaaagcagatccgagcgcacgttttgaagttaccctggttgttcgtcagcctgcacaggatgcatttgcacgtcatctggaagcactgcatgatgttacccgtcgtcctccggcactgacccgtgaagcctatgcagcacagtatagcgcagcagcagatgattttgcagcagttgaacagtttgcagcaagcgaaggtctgcaggttgtgcgtcgtgatgcagcccagcgtaccattgttctgagcggcaccgttgcacagtttaatcatgcatttgaaatcgatctgcagaagattgaacacgagggtaaaagctatcgtggtcgtgttggtccggttcatctgccgcagcatctgaaaaccgttgttgatgcagttctgggtttagaagatctgccgctggcacgtacccattttcgtctgcagcctgcagcacgtagcgcagccggttttacaccgctggaactggcaagcatttatcagtttccggcaggcgcaggtaaaggtcaggccattgcactgattgaattaggtggtggtgttaaaaccagcgatctgaccacctattttagccagctgggtgttacccctccgcaggttaccgcagttagcgttgatcaggcaaccaatagtccgaccggtgatccgaatggtccggatggtgaagtgacactggatgttgaaattaccggtgcaattgcccctgaagcacatattgttctgtattttgcaccgaataccgaagccggtttctttaatgcagtttcagcagcagttcatgataccacacatcgtccgaccgttattagcattagctggggtggtccggaagcagcatggacccgtcagagcctggatgcctttgatcgtgcactgcaggcagccgcagcaatgggtgtgaccgtttgtgcagccagcggtgatagcggtagcagcggtagtcctggtaatggttcaccgcaggttgattttccggcaagcagtccgcatgttctggcatgtggtggcacccgtctgcatgcaagcgcaaatcgccgtgatgccgaaagcgtttggaatgatggtgcaggcggtggtgcaagtggtggtggcgttagcgcagcgtttgcactgccgagctggcaagagggcctgcaggttacagccgcagatggcaccagccaggcgctgacccagcgtggtgttccggatgttgccggtgatgcaagtccggcaagtggttatgatgttgttgtggatgcacaggccaccattgttggtggtacaagcgcagttgcaccgctgtgggcaggtctgattgcacgtctgaatgccagcctgggtaaaccgctgggttatctgaatccgattctgtatcagcatccgggtgttctgaatgatatcacccagggcgataatggtgaatttagtgcagcacctggttgggatgcatgtaccggtctgggtagcccgaatggccagaaaattgcgggtgttgcatga

SEQ ID NO: 12 - protein, 514 aa, Pandoraea thiooxydans
MPTFLLPGSEQTCPPGARCVGKADPSARFEVTLVVRQPAQDAFARHLEALHDVTRRPPALTREAYAAQYSAAADDFAAVEQFAASEGLQVVRRDAAQRTIVLSGTVAQFNHAFEIDLQKIEHEGKSYRGRVGPVHLPQHLKTVVDAVLGLEDLPLARTHFRLQPAARSAAGFTPLELASIYQFPAGAGKGQAIALIELGGGVKTSDLTTYFSQLGVTPPQVTAVSVDQATNSPTGDPNGPDGEVTLDVEITGAIAPEAHIVLYFAPNTEAGFFNAVSAAVHDTTHRPTVISISWGGPEAAWTRQSLDAFDRALQAAAAMGVTVCAASGDSGSSGSPGNGSPQVDFPASSPHVLACGGTRLHASANRRDAESVWNDGAGGGASGGGVSAAFALPSWQEGLQVTAADGTSQALTQRGVPDVAGDASPASGYDVVVDAQATIVGGTSAVAPLWAGLIARLNASLGKPLGYLNPILYQHPGVLNDITQGDNGEFSAAPGWDACTGLGSPNGQKIAGVA

SEQ ID NO: 13 - DNA, 1700 bp, synthetic construct
  misc_feature 1..1700: note = Description of Artificial Sequence: Synthetic polynucleotide
  source 1..1700: mol_type = other DNA
gaaataattttgtttaactttaagaaggagatatacatatgcgccatcgttttggtctgagcattctgtttctggttctggtgagcagcgcagttgcacaggttattgttccgcctaccagcgttcgtcgtccgggtgaacgtccgggtacagcacataccaattatcgtatctatattggtccgtggcgttttccgagcgttgatagcccgtttccggaactggcagcagcacatggtccggcagcaggtcagaccattccgggttatcatccggcagatattcgtgcagcatataatgttcctccgaatctgggcacccaggccattgcaattgttgatgcatttgatctgccgaccagcctgaatgattttaacttttttagcgcacagtttggcctgccgaccgaaccgagcggtgttgcaaccgcaagcaccaatcgtgtttttcaggttgtttatgcaagcggcaccaaaccggcaaccaatgcagattggggtggtgaaattgcactggatattgaatgggcacatgcaatggcaccgaatgcaaaaatctatctgattgaagcagatagcgatagcctgctggatctgctggcagccgttcgtgttgcagcaacccagctgagcaatgttcgtcagattagcatgagctttggtgccaatgaatttaccaatgaaagcgcaagcgatagcacctttctgggtacaaataaagttttttttgccagcagcggtgatgcaagcaatctggttagctatccggcagcgagcccgaatgttgttggtgttggtggcacccgtctggcactgagtaatggtagcgttgttagcgaaaccgcatggtcaagtgccggtggtggtccgagcagccgtgaaccgcgtccgacctatcagaatagcgttagcggtgtggttggtagcgcacgtggtacaccggatattgcagcaattgcagatccggaaaccggtgttgccgtttatgatagcaccccgattccaggtacaggtgttggttggtttgttgttggcggtacaagcctggcatgtccggtttgtgcaggtattaccaatgcacgtggttattttaccgccagcagctttagcgaactgacccgtctgtatggtctggcaggcaccagcttttttcgtgacattaccagcggcacctcaggtcagtttagtgcacgtgttggttatgattttgttaccggtctgggtagtctgctgggtatttttggtccgtttgcaaccagtccgagtagcctgagcgttgtgagcggcaccgcagttgccggtgttccgagcaatatggttgccaaagatggtcatgattatgttgttcgtagcgcaagtccggcaggcggtggtcaggttgccaccgttcagggcacctttgcaagccatccgcctgcaaaagcagttcagtttggtgcaagcgttaccgttaccgcaatgcgtaccagcggtacaaccacactgaaactgtttaatcaggcaaccagcgcatttgaaagcgttgcaaatctgaccctgggcaccaccaataccaccgtgaccgttccgattccgaatgcaccgaaatactttgcaagtgatggtacgaccaaatttcagctgaccaccacaggtcctggtacaacacagattcgctttggtgttgatcaggttctgctgaccctgacaccgacaggctga

SEQ ID NO: 14 - protein, 553 aa, Fimbriimonas ginsengisoli
MRHRFGLSILFLVLVSSAVAQVIVPPTSVRRPGERPGTAHTNYRIYIGPWRFPSVDSPFPELAAAHGPAAGQTIPGYHPADIRAAYNVPPNLGTQAIAIVDAFDLPTSLNDFNFFSAQFGLPTEPSGVATASTNRVFQVVYASGTKPATNADWGGEIALDIEWAHAMAPNAKIYLIEADSDSLLDLLAAVRVAATQLSNVRQISMSFGANEFTNESASDSTFLGTNKVFFASSGDASNLVSYPAASPNVVGVGGTRLALSNGSVVSETAWSSAGGGPSSREPRPTYQNSVSGVVGSARGTPDIAAIADPETGVAVYDSTPIPGTGVGWFVVGGTSLACPVCAGITNARGYFTASSFSELTRLYGLAGTSFFRDITSGTSGQFSARVGYDFVTGLGSLLGIFGPFATSPSSLSVVSGTAVAGVPSNMVAKDGHDYVVRSASPAGGGQVATVQGTFASHPPAKAVQFGASVTVTAMRTSGTTTLKLFNQATSAFESVANLTLGTTNTTVTVPIPNAPKYFASDGTTKFQLTTTGPGTTQIRFGVDQVLLTLTPTG

SEQ ID NO: 15 - DNA, 1697 bp, synthetic construct
  misc_feature 1..1697: note = Description of Artificial Sequence: Synthetic polynucleotide
  source 1..1697: mol_type = other DNA
gaaataattttgtttaactttaagaaggagatatacatatgagcgatatggaaaaaccgtggaaagaagaagaaaaacgcgaagttctggcaggtcatgcacgtcgtcaggcaccgcaggcagttgataaaggtccggttaccggtgatcagcgtattagcgttaccgttgttctgcgtcgtcagcgtggtgatgaactggaagcacatgttgaacgtcaggcagcactggcaccgcatgcacgtgttcatctggaacgtgaagcatttgcagcaagccatggtgcaagcctggatgattttgcagaaattcgtaaatttgccgaagcgcatggtctgaccctggatcgtgcccatgttgcagcaggtacagcagttctgagcggtccggttgatgcagttaatcaggcatttggtgttgaactgcgtcattttgatcatcctgatggtagctatcgtagctatgttggtgatgttcgtgttccggcaagcattgcaccgctgattgaagcagttttaggtctggatacccgtccggttgcacgtccgcattttcgtctgcgtcgccgtgcagaaggtgaatttgaagcacgtagccagagcgcagcaccgaccgcatatacaccgctggatgttgcacaggcatatcagtttccggaaggcctggatggtcagggtcagtgtattgcaattattgaattaggtggtggctatgatgaaaccagcctggcacagtattttgccagcctgggtgttagcgctccgcaggttgttagcgttagcgtggatggtgcaaccaatcagccgacaggtgatccgaatggtccggatggtgaagttgaactggatattgaagttgccggtgcgctggcaccgggtgcaaaaattgcagtttattttgcaccgaataccgatgccggttttctgaatgcaattaccaccgcagttcatgatccgacacataaaccgagcattgtgagcattagctggggtggtccggaagatagctgggcaccagccagcattgcagccatgaatcgtgcatttctggatgcagccgcactgggtgtgaccgtgctggcagcagccggtgatagcggtagcaccgatggtgaacaggatggtctgtatcatgttgattttccggcagcgagcccgtatgttctggcatgtggtggcacccgtctggtggcaagcgcaggtcgtattgaacgtgaaaccgtttggaatgatggtcctgatggcggttcaaccggtggtggtgttagccgtatttttccgctgccgagctggcaagaacgtgcaaatgttccgcctagcgcaaatcctggtgcaggtagcggtcgtggtgttccggatgttgccggtaatgcagatccggcaaccggttatgaagttgttattgatggtgaaaccaccgtgattggtggtacaagcgcagtggcaccgctgtttgcagccctggttgcccgtattaatcagaaactgggtaaaccggttggttatctgaatccgacactgtatcagctgcctccggaagtttttcatgatattaccgaaggcaacaacgatattgccaatcgtgcacgtatttatcaggcaggtcctggttgggatccgtgtaccggtctgggtagcccgattggtattcgtctgctgcaggcactgctgccgagtgcaagccaggcacagccgtga

SEQ ID NO: 16 - protein, 552 aa, Bacillus sp.
MSDMEKPWKEEEKREVLAGHARRQAPQAVDKGPVTGDQRISVTVVLRRQRGDELEAHVERQAALAPHARVHLEREAFAASHGASLDDFAEIRKFAEAHGLTLDRAHVAAGTAVLSGPVDAVNQAFGVELRHFDHPDGSYRSYVGDVRVPASIAPLIEAVLGLDTRPVARPHFRLRRRAEGEFEARSQSAAPTAYTPLDVAQAYQFPEGLDGQGQCIAIIELGGGYDETSLAQYFASLGVSAPQVVSVSVDGATNQPTGDPNGPDGEVELDIEVAGALAPGAKIAVYFAPNTDAGFLNAITTAVHDPTHKPSIVSISWGGPEDSWAPASIAAMNRAFLDAAALGVTVLAAAGDSGSTDGEQDGLYHVDFPAASPYVLACGGTRLVASAGRIERETVWNDGPDGGSTGGGVSRIFPLPSWQERANVPPSANPGAGSGRGVPDVAGNADPATGYEVVIDGETTVIGGTSAVAPLFAALVARINQKLGKPVGYLNPTLYQLPPEVFHDITEGNNDIANRARIYQAGPGWDPCTGLGSPIGIRLLQALLPSASQAQP

SEQ ID NO: 17 - DNA, 1805 bp, synthetic construct
  misc_feature 1..1805: note = Description of Artificial Sequence: Synthetic polynucleotide
  source 1..1805: mol_type = other DNA
gaaataattttgtttaactttaagaaggagatatacatatgatgaaaagcagcgcagcaaaacagaccgttctgtgtctgaatcgttatgcagttgttgcactgccgctggcaattgcaagctttgcagcatttggtgcaagtccggcaagcaccctgtgggcaccgaccgataccaaagcatttgttacaccggcacaggttgaagcacgtagcgcagcaccgctgctggaactggcagccggtgaaaccgcacatattgttgttagcctgaaactgcgtgatgaagcacagctgaaacagctggcacaggcagttaatcagcctggtaatgcacagtttggcaaatttctgaaacgtcgtcagtttctgagccagtttgcaccgacagaagcacaggttcaggccgttgttgcccatctgcgtaaaaatggttttgtgaacattcatgttgtgccgaatcgtctgctgattagcgcagatggtagtgccggtgcagttaaagcagcatttaatacaccgctggttcgttatcagctgaatggtaaagcaggttatgcaaataccgcaccagcgcaggttccgcaggatctgggtgaaattgttggtagcgttctgggtctgcagaatgttacccgtgcacatccgatgctgaaagttggtgaacgtagtgcagcaaaaaccctggcagcaggcaccgcaaaaggtcataatccgaccgaatttccgaccatttatgatgccagcagcgctccgaccgcagcaaataccaccgtgggtattattaccattggtggtgttagtcagaccctgcaagatctgcagcagtttaccagcgcaaatggtctggcaagcgttaatacccagacaattcagaccggtagcagcaatggtgattattcagatgatcagcaaggtcaaggtgaatgggatttagatagccagagcattgttggttcagccggtggtgcagttcagcaactgctgttttatatggcagatcagagcgccagcggtaatacaggtctgacccaggcctttaatcaggcggttagcgataatgttgccaaagttattaatgtgagcttaggttggtgtgaagcagatgcaaatgcagatggcaccctgcaggcagaagatcgtatttttgcaaccgcagcagcccagggccagacctttagcgttagcagtggtgatgaaggtgtttatgaatgcaataatcgtggttatccggatggtagcacctatagcgtgagctggcctgcaagcagcccgaatgttattgccgttggtggtacaaccctgtataccaccagtgcgggtgcatatagcaatgaaaccgtttggaatgaaggtctggatagcaatggcaaactgtgggcaaccggtggtggttatagcgtgtatgaaagcaaaccgagctggcagagcgttgttagcggtacaccgggtcgccgtctgctgccggatattagctttgatgcagcacaaggtacaggtgcactgatttataactatggtcagctgcagcagattggtggcaccagcctggcaagcccgatttttgttggtttatgggcacgtctgcagagcgcaaatagcaatagcctgggttttccggcagccagcttttatagcgcaattagcagcaccccgagcctggttcatgatgttaaatcaggtaataatggctatggtggctacggttataatgccggtacaggttgggattatccgaccggttggggtagcctggatattgcaaaactgagcgcatatattcgtagcaacggttttggtcattga

SEQ ID NO: 18 - protein, 588 aa, Pseudomonas sp.
MMKSSAAKQTVLCLNRYAVVALPLAIASFAAFGASPASTLWAPTDTKAFVTPAQVEARSAAPLLELAAGETAHIVVSLKLRDEAQLKQLAQAVNQPGNAQFGKFLKRRQFLSQFAPTEAQVQAVVAHLRKNGFVNIHVVPNRLLISADGSAGAVKAAFNTPLVRYQLNGKAGYANTAPAQVPQDLGEIVGSVLGLQNVTRAHPMLKVGERSAAKTLAAGTAKGHNPTEFPTIYDASSAPTAANTTVGIITIGGVSQTLQDLQQFTSANGLASVNTQTIQTGSSNGDYSDDQQGQGEWDLDSQSIVGSAGGAVQQLLFYMADQSASGNTGLTQAFNQAVSDNVAKVINVSLGWCEADANADGTLQAEDRIFATAAAQGQTFSVSSGDEGVYECNNRGYPDGSTYSVSWPASSPNVIAVGGTTLYTTSAGAYSNETVWNEGLDSNGKLWATGGGYSVYESKPSWQSVVSGTPGRRLLPDISFDAAQGTGALIYNYGQLQQIGGTSLASPIFVGLWARLQSANSNSLGFPAASFYSAISSTPSLVHDVKSGNNGYGGYGYNAGTGWDYPTGWGSLDIAKLSAYIRSNGFGH

SEQ ID NO: 19 - DNA, 1673 bp, synthetic construct
  misc_feature 1..1673: note = Description of Artificial Sequence: Synthetic polynucleotide
  source 1..1673: mol_type = other DNA
gaaataattttgtttaactttaagaaggagatatacatatggccaacggtaaaagcaccagtccggcaagccagtgggttccgctgcctggtagcaatcgtcagctgctgccgcagagcgttccgattggtccggcagatctgaaagcaaccgttgcactgaccgttaaagttcgtagccgtggtaaactggcagaactggatgatgcagttaaaaaagaaagcgcaaaaccgctgaaagaacgcacctatattagccgtgaagaactggcacagcgttatggtgcagatgcagatgatctggataaagttgaactgtatgccaacaaacatcatctgcgtgttgcagatcgtgatgaagcaacccgtcgtgttgttctgaaaggcaccctggaagatgcactgagcgcatttcatgcagatgttcacatgtatcagcatgcaagcggtccgtatcgtggtcgtcgtggtgaaattctggttcctgcagaactgaaagatgttgtgaccggtatttttggctttgatacccatccgaaacatcgtgcaccgcgtcgtctgatgggcaccagcagcggcaccgcaaccaatctgggtgaatttgcaagcgaatttgcgacccgttatcagtttccgaccagcagcagcagtaccaaactggatggcaccggtcagtgtattgcactgattgaattaggtggtggctatagcaataacgatctgaaaatcttttttagcgaagccggtgttccgatgccgaaagttgttgcagttagcattgatcatggtgcaaatcatccgacaccgcaaggtctggcagatggtgaagttatgctggatattgaagttgccggtgttgttgcaccgggtgccaaactggccgtttattttgcaccgaatagcgatagcggttttcaggatgcaattcgtgcagcagttcatgatggtgcacgtaaaccgagcgttgttagcattagctggggtgaacctgatgattttctgaccgcacagagcgtgcagagctatcatgaaatctttaccgaagcagcagccctgggtgttaccgtttgtgcagcaagcggtgatcatggcgttgccgatctggatgcactgcattgggataaacgtattcatgttaatcatccgtcaagcgatccgctggttctgtgttgtggtggtacacagattgataaaaatgttgatgtggtgtggaatgatggcaccccgtttgatccgcaggtttttggtggtggcggttgggccagcggtggtggtattagtccggtgtttggtgttccggattatcagaaaggtctgccgatgccgtcaagcctgagcaccagccagcctggtcgtggttgtccggatattgcaatgaccgcagataactatcgtacccgtgttcatggtgttgatggtccgagcggtggcaccagcgcagttacaccgctgatggcatgtctggttgcacgtctgaatcaggcatttgaaaaaaatctgggttttgtgaatccgctgctgtatgcaaatgcacaggcatttaccgatattacccagggcaccaatggtattaatcagaccattgaaggttatccggcaggtaaaggttgggatgcatgtaccggtctgggtgcaccgattggcaccgttctgctgcaggcactgggtaaatga

SEQ ID NO: 20 - protein, 544 aa, Variovorax sp.
MANGKSTSPASQWVPLPGSNRQLLPQSVPIGPADLKATVALTVKVRSRGKLAELDDAVKKESAKPLKERTYISREELAQRYGADADDLDKVELYANKHHLRVADRDEATRRVVLKGTLEDALSAFHADVHMYQHASGPYRGRRGEILVPAELKDVVTGIFGFDTHPKHRAPRRLMGTSSGTATNLGEFASEFATRYQFPTSSSSTKLDGTGQCIALIELGGGYSNNDLKIFFSEAGVPMPKVVAVSIDHGANHPTPQGLADGEVMLDIEVAGVVAPGAKLAVYFAPNSDSGFQDAIRAAVHDGARKPSVVSISWGEPDDFLTAQSVQSYHEIFTEAAALGVTVCAASGDHGVADLDALHWDKRIHVNHPSSDPLVLCCGGTQIDKNVDVVWNDGTPFDPQVFGGGGWASGGGISPVFGVPDYQKGLPMPSSLSTSQPGRGCPDIAMTADNYRTRVHGVDGPSGGTSAVTPLMACLVARLNQAFEKNLGFVNPLLYANAQAFTDITQGTNGINQTIEGYPAGKGWDACTGLGAPIGTVLLQALGK

SEQ ID NO: 21 - DNA, 1581 bp, synthetic construct
  misc_feature 1..1581: note = Description of Artificial Sequence: Synthetic polynucleotide
  source 1..1581: mol_type = other DNA
gaaataattttgtttaactttaagaaggagatatacatatgaaaaccagcaacaaagttgcactggcaggtagctacaaaaaagcacatagcggtgaaaccaccgccaaaattaaccgtaatacctttattgaagtgaccctgcgtattcgtcgcaaaaaaagcattgaaagcctgctgaatgcaggtaaacgtgttgatcatgccgattacgaaaaagaatttggtgcaagccagaaagatgcagatcaggttgaagcatttgcacgtcagtataaactgagcaccgttgaagttagcctgagccgtcgtagcgttattctgcgtggtagcattgcaaatatggaagcagcatttgatgtgaatctgagcaaagcagttgatagccatggtgatgatattcgtgttcgtaaaggcgatatctatattccggaagcactgaaagatgttgtggaaggtgtttttggtctggataatcgtaaagcagcacgtccgctgtttaaactgctgaaaaaagcagatggtattagtccgcaggcaagcgttagcagcagctttaccccgaatcagctggcaggcatttatggttttccggcaggttttaatggtaaaggtcagaccattgccattattgaattaggtggtggttatcgtaccaccgatctgaccaattatttcaaaaaactgggcatcaaaaaaccgtccattaaagccattctggtggacaaaggtaaaaacaatccgagcaatgcaaatagcgcagatggtgaagttatgctggatattgaagttgccggtgcagttgcaagcggtgcaaaaattgttgtgtattttagcccgaataccgacaaaggttttctggatgcaattaccaaagccgttcatgataccacacataaaccgagcgttgttagcattagctggggtggtggtgaagcagtttggacccagcagagcctgaatagttttaatgaagcctttaaagcagccgcagttctgggtgttaccgtttgtgcagcagccggtgataatggtagcagtgatggcctgaccgataatagcgttcatgttgattttccagcaagcagcccgtatgttctggcatgtggtggtacaaccctgaaagtgaaaaacaatgttattaccagcgaaaccgtttggcatgatagcaatgatagcgcaaccggtggtggcgttagcaatgtttttccgctgccggattatcagaaaaatgccggtgttccggcagcaattggcaccaactttattggtcgtggtgtgccggatgttgcaggtaatgcagatccgaatacaggttataatgttctggttgatggtcagcagctggttattggtggcaccagcgcagtggcaccgctgtttgcaggtctgattgcatgtctgaatcagaaaagcggtaaatggtcaggttttatcaatccgacactgtatgcagcaaatccgagcgtttgtcgtgatattaccgttggtaataatcgtaccgccaccggtaatgccggttatgatgcacgtgttggttgggatccgtgtaccggtctgggtgtgtttagcaaactgctga

SEQ ID NO: 22 - protein, 513 aa, Mucilaginibacter sp.
MKTSNKVALAGSYKKAHSGETTAKINRNTFIEVTLRIRRKKSIESLLNAGKRVDHADYEKEFGASQKDADQVEAFARQYKLSTVEVSLSRRSVILRGSIANMEAAFDVNLSKAVDSHGDDIRVRKGDIYIPEALKDVVEGVFGLDNRKAARPLFKLLKKADGISPQASVSSSFTPNQLAGIYGFPAGFNGKGQTIAIIELGGGYRTTDLTNYFKKLGIKKPSIKAILVDKGKNNPSNANSADGEVMLDIEVAGAVASGAKIVVYFSPNTDKGFLDAITKAVHDTTHKPSVVSISWGGGEAVWTQQSLNSFNEAFKAAAVLGVTVCAAAGDNGSSDGLTDNSVHVDFPASSPYVLACGGTTLKVKNNVITSETVWHDSNDSATGGGVSNVFPLPDYQKNAGVPAAIGTNFIGRGVPDVAGNADPNTGYNVLVDGQQLVIGGTSAVAPLFAGLIACLNQKSGKWSGFINPTLYAANPSVCRDITVGNNRTATGNAGYDARVGWDPCTGLGVFSKL

SEQ ID NO: 23 - DNA, 1724 bp, synthetic construct
  misc_feature 1..1724: note = Description of Artificial Sequence: Synthetic polynucleotide
  source 1..1724: mol_type = other DNA
gaaataattttgtttaactttaagaaggagatatacatatggcaccgaaaaccagcgttccgcattttaccacacagagccgtaccgttctgagcggtagcgaaaaagcaccggttgccgaagcacgtggtgcaaaaccggcaccgctggcagcacgtattaccgttagcgttattgttcgtcgtaaaacaccgctgaaagcagcccatattaccggtgaacagcgtctgacccgtgcacagtttaatgcaagccatgcagcagatccggcagcagttaaactggttcagggttttgccaaagaatttggtctgaccgttgatccgggtactccggcaccgggtcgtcgtaccatgaaactgaccggtacagtggcaaatatgcagcgtgcatttggtgttagcctggcacataaaaccatggatggtgttacctatcgtgttcgtgaaggtagcattaatctgcctgcagaactgcagggttatgttgttgcagttttaggtctggataatcgtccgcaggcagaaccgcattttcgtattctgggtgaacagggtgcagttgcagcacaggcagcacaaggtcagggctttgcaggtccgcatgccggtggtagcaccagctatacaccggttcaggttggtgaactgtatcagtttccgcgtggtagcagcgcaagcaatcagaccattggtattattgaattaggtggtggttttcgccagaccgatattgcagcatactttaaaaccctgggtcagaaaccgcctcaggttattgcagttccgattggtaatggtaaaaacaatccgaccaatagcaatagcgcagatggtgaagttatgctggatattgaagttgccggtgccgttgcaccgggtgcacgtattgttgtttattttgcaccgaataccgatcagggtttcgttgatgcaattgcccatgcaattcatgataccacctataaaccgagcgttattagcattagctggggtagcgcagaagttaattggaccgttcaggcaatggcagcactggatgcagcatgtcagagcgcagcagccctgggtattacaattaccgcagcaagcggtgataatggtagcagtgatgcagttgccgatggtgaaaatcatgttgattttccggcaagcagtccgcatgttctggcatgtggtggcaccaatctgcaaggtagcggtagtaccattagtgcagaaaccgtttggaatgcacagccgcaaggtggtgcgaccggtggtggtgtgagcaacatttttccgctgccgacctggcaggcaagcagcaaagttccgaaaccgacacatccgagcggtggtcgtggtgttccggatgttgcgggtgatgccgatccggcaagtggttatgtggttcgtgttgatggtcagacctttgttattggtggtacaagcgcagttgcaccgctgtgggcaggcctgattgcagttgcgaatcagcagaatggtaaatcagcaggttttattcagcctgcaatttatgcaggtcagggtaaaccggcatttcgtgataccgtgcagggtagcaatggtagctttgcagcaggcgcaggttgggatgcatgcaccggtctgggtagcccgattgcactgcagctgattaacgcaatcaaaccggcaagctcaaaaagcaaaagcaaagcgattgcagcaaaacgcaaaaccattatccgtaccaaaaaatga

SEQ ID NO: 24 - protein, 561 aa, Bradyrhizobium erythrophlei
MAPKTSVPHFTTQSRTVLSGSEKAPVAEARGAKPAPLAARITVSVIVRRKTPLKAAHITGEQRLTRAQFNASHAADPAAVKLVQGFAKEFGLTVDPGTPAPGRRTMKLTGTVANMQRAFGVSLAHKTMDGVTYRVREGSINLPAELQGYVVAVLGLDNRPQAEPHFRILGEQGAVAAQAAQGQGFAGPHAGGSTSYTPVQVGELYQFPRGSSASNQTIGIIELGGGFRQTDIAAYFKTLGQKPPQVIAVPIGNGKNNPTNSNSADGEVMLDIEVAGAVAPGARIVVYFAPNTDQGFVDAIAHAIHDTTYKPSVISISWGSAEVNWTVQAMAALDAACQSAAALGITITAASGDNGSSDAVADGENHVDFPASSPHVLACGGTNLQGSGSTISAETVWNAQPQGGATGGGVSNIFPLPTWQASSKVPKPTHPSGGRGVPDVAGDADPASGYVVRVDGQTFVIGGTSAVAPLWAGLIAVANQQNGKSAGFIQPAIYAGQGKPAFRDTVQGSNGSFAAGAGWDACTGLGSPIALQLINAIKPASSKSKSKAIAAKRKTIIRTKK

SEQ ID NO: 25 - protein, 523 aa, synthetic construct
  REGION 1..523: note = Description of Artificial Sequence: Synthetic polypeptide
MSEPVPAAARRTIPGSERPPVDTAAAARQAVPADTRVEATVVLRRRAELPDGPGLLTPAELAERHGADPADVELVTRTLTGLGVEVTAVDAASRRLRVAGPAGVLAEAFGTSLAQVSTPDPSGAQVTHRYRAGALSVPAELDGVVTAVLGLDDRPQARARFRVATAAAASAGYTPIELGRVYSFPEGSDGSGQTIAIIELGGGFAQSELDTYFAGLGISGPTVTAVGVDGGSNVAGRDPQGADGEVLLDIEVAGALAPGADVVVYFAPNTDAGFLDAVAQAAHATPTPAAISISWGGSEDTWTGQARTAFDAALADAAALGVTTTVAAGDDGSTDRATDGKSHVDFPASSPHALACGGTHLDANATTGAVTSEVVWNNGAGKGATGGGVSTVFAQPSWQASAGVPDGPGGKPGRGVPDVSAVADPQTGYRIRVDGQDLVIGGTSAVAPLWAALVARLVQAGRAKLGLLQPKLYAAPTAFRDITEGDNGAYRAGPGWDACTGLGVPVGTALASALSLEHHHHHH

SEQ ID NO: 26 - protein, 539 aa, synthetic construct
  REGION 1..539: note = Description of Artificial Sequence: Synthetic polypeptide
MADDSSPTTAADRPTLPGSARRPVAAAQAAGPLDDAAPLEVTLVLRRRTALPAGTGRPAPMGRAEFAETHGADPADAETVTAALTAEGLRITAVDLPSRRVQVAGDVATFSRVFGVSLSRVESPDPVADRLVPHRQRSGDLAVPAPLAGVVTAVLGLDDRPQARALFRPAAAVDTTFTPLELGRVYRFPSGTDGRGQRLAILELGGGYTQADLDAYWTTIGLADPPTVTAVGVDGAANAPEGDPNGADGEVLLDIEVAGALAPGADLVVYFAPNTDRGFLDALSTAVHADPTPTAVSISWGQNEDEWTAQARTAMDEALADAAALGVTVCAAAGDDGSTDNAPDGQAHVDFPASSPHALACGGTTLRADPDTGEVSSETVWFHGTGQGGTGGGVSAVFAVPDWQDGVRVPGDADTGRHGRGVPDVSADADPSTGYQVRVDGTDAVFGGTSAVSPLWSALTCRLAEALGQRPGLLQPLIYAGLSAGEVAAGFRDVTSGSNGAYDAGPGWDPCTGLGVPDGEALLVRLRTALGLEHHHHHH

SEQ ID NO: 27 - protein, 532 aa, synthetic construct
  REGION 1..532: note = Description of Artificial Sequence: Synthetic polypeptide
MDYQILRGSERSPLPGCTDTGKFPAAHRLRVLLALRQPELDAAAARLLDTAGDELPAPLSRDAFATRFAAAADDLRAVEAFATQHGLSMEQTLAHAGVAILEGSVQQFDRAFQVDLRDYRKDDLRYRGRTGAVSIPTALHGVVSAVLGLDDRPQAHTLPQAQDAPAPAGAAAPIARYTPPQLAELYGFPEHDGAGQCIGIIALGGGYERAQLAAYFTELGLPMPQIVDVLLAGARNQPGGQGRKADIEVQMDVQIAGAIAPGAKLVVYFAPNTDNGFLEAIVSAIHDRAHAPDVIAISWGFTETLWTAQSRAAYNRALQAAALMGITVCIASGDDGASDGQPGLNVCFPASSPFVLACGGTRLQVDVQAQHEQAWSGTGGGQSRVFARPRWQQALTLHGTQQTAQPLSMRGVPDVAANADAETGYYVHIDGRPAVMGGTSAAAPVWAALLARVYGLNGGRRVFLPPRLYAVADVCRDIVDGGNGGFVASPGWDACTGLGVPDGGRIAAALGAGPGAKPAITPTGLEHHHHHH

SEQ ID NO: 28 - protein, 558 aa, synthetic construct
  REGION 1..558: note = Description of Artificial Sequence: Synthetic polypeptide
MTRHPVSDSGASNEHPVPAGAQCMGACDPAEHFNVVVIVRRQSERAFRELVERIATGAPGAQPISREQYEQRFSADAADVARVEAFAKTHGLVVVKADRDTRRVVLSGTVQQYNAAFGVDLQRFEHQVGKLKQHFRQPTGPVHLPEDLHEVITAVVGLDSRAKVQPHFRIDSQTPATPPEKASQPGDGVVHAPIRAARAVSRSFTPLQLAELYDFPPGDGKGQCIALIEMGGGYAQSDLDAYFSALGVTRPRVEAVSVDQATNAPSGDPNGPDAEVTLDVEIAGALAPGALIAVYFAPNSEAGFVDAVSAALHDSQRKAAIISISWGAPESIWSQQTLGALNDALQTAVALGVTVCCASGDSGSSDGVTDGADHVDFPASSPYALGCGGTQLTAANGRITRETVWGSGANGATGGGVSATFAVPAWQKGLKVSRGSGAARALALARRGVPDVAADADPATGYEVHIGGMDTVVGGTSAVAPLWAALVARINAGSGKAAGFINAKLYARPGAFNDITSGSNGDYAARPGWDACTGLGTPVGTRVAAAIGSALEHHHHHH

SEQ ID NO: 29 - protein, 540 aa, synthetic construct
  REGION 1..540: note = Description of Artificial Sequence: Synthetic polypeptide
MVRHPLRGSERTIPEDARILGDAHPAEQIRALVQLRRPNEAELDVRLSGFVHAHAAGTPSPTPLTREEWAAQFGAATDDIDAVRTFAREHGLQVAEVNVAAATVMLEGSVEQFCRAFDTHLHRVAHGGSEYRGRSGPLRLPESLQDVVVAVLGLDSRPQAAPHFRFVPLPTGSVEPGGIRPARAAPTASYTPVQLAQLYGFPQGDGAGQCIAFVELGGGYREDDLRAYFQEVGMPMPTVTAIPVGQGANRPTGDPSGPDGEVMLDLEVAGAAAPGATLAVYFTVNTDAGFVQAINAAIHDTKLRPSVVSISWGAPESAWTPQAMQAVNAALQSAATMGVTVCAASGDSGSSDGQPDRVDHVDFPASSPYALACGGTSVRASGNRIAEETVWNDGARGGAGGGGVSTVFALPSWQQGLAAQQTGGDSVPLARRGVPDVSADADPLTGYVVRVDGESGVVGGTSAAAPLWAALIARINAIKGRPAGYLHARLYQNPGAFNDIKQGNNGAFAAAPGWDACTGLGSPKGDAIANLFLEHHHHHH

SEQ ID NO: 30 - protein, 522 aa, synthetic construct
  REGION 1..522: note = Description of Artificial Sequence: Synthetic polypeptide
MPTFLLPGSEQTCPPGARCVGKADPSARFEVTLVVRQPAQDAFARHLEALHDVTRRPPALTREAYAAQYSAAADDFAAVEQFAASEGLQVVRRDAAQRTIVLSGTVAQFNHAFEIDLQKIEHEGKSYRGRVGPVHLPQHLKTVVDAVLGLEDLPLARTHFRLQPAARSAAGFTPLELASIYQFPAGAGKGQAIALIELGGGVKTSDLTTYFSQLGVTPPQVTAVSVDQATNSPTGDPNGPDGEVTLDVEITGAIAPEAHIVLYFAPNTEAGFFNAVSAAVHDTTHRPTVISISWGGPEAAWTRQSLDAFDRALQAAAAMGVTVCAASGDSGSSGSPGNGSPQVDFPASSPHVLACGGTRLHASANRRDAESVWNDGAGGGASGGGVSAAFALPSWQEGLQVTAADGTSQALTQRGVPDVAGDASPASGYDVVVDAQATIVGGTSAVAPLWAGLIARLNASLGKPLGYLNPILYQHPGVLNDITQGDNGEFSAAPGWDACTGLGSPNGQKIAGVALEHHHHHH

SEQ ID NO: 31 - protein, 561 aa, synthetic construct
  REGION 1..561: note = Description of Artificial Sequence: Synthetic polypeptide
MRHRFGLSILFLVLVSSAVAQVIVPPTSVRRPGERPGTAHTNYRIYIGPWRFPSVDSPFPELAAAHGPAAGQTIPGYHPADIRAAYNVPPNLGTQAIAIVDAFDLPTSLNDFNFFSAQFGLPTEPSGVATASTNRVFQVVYASGTKPATNADWGGEIALDIEWAHAMAPNAKIYLIEADSDSLLDLLAAVRVAATQLSNVRQISMSFGANEFTNESASDSTFLGTNKVFFASSGDASNLVSYPAASPNVVGVGGTRLALSNGSVVSETAWSSAGGGPSSREPRPTYQNSVSGVVGSARGTPDIAAIADPETGVAVYDSTPIPGTGVGWFVVGGTSLACPVCAGITNARGYFTASSFSELTRLYGLAGTSFFRDITSGTSGQFSARVGYDFVTGLGSLLGIFGPFATSPSSLSVVSGTAVAGVPSNMVAKDGHDYVVRSASPAGGGQVATVQGTFASHPPAKAVQFGASVTVTAMRTSGTTTLKLFNQATSAFESVANLTLGTTNTTVTVPIPNAPKYFASDGTTKFQLTTTGPGTTQIRFGVDQVLLTLTPTGLEHHHHHH

SEQ ID NO: 32 - protein, 560 aa, synthetic construct
  REGION 1..560: note = Description of Artificial Sequence: Synthetic polypeptide
MSDMEKPWKEEEKREVLAGHARRQAPQAVDKGPVTGDQRISVTVVLRRQRGDELEAHVERQAALAPHARVHLEREAFAASHGASLDDFAEIRKFAEAHGLTLDRAHVAAGTAVLSGPVDAVNQAFGVELRHFDHPDGSYRSYVGDVRVPASIAPLIEAVLGLDTRPVARPHFRLRRRAEGEFEARSQSAAPTAYTPLDVAQAYQFPEGLDGQGQCIAIIELGGGYDETSLAQYFASLGVSAPQVVSVSVDGATNQPTGDPNGPDGEVELDIEVAGALAPGAKIAVYFAPNTDAGFLNAITTAVHDPTHKPSIVSISWGGPEDSWAPASIAAMNRAFLDAAALGVTVLAAAGDSGSTDGEQDGLYHVDFPAASPYVLACGGTRLVASAGRIERETVWNDGPDGGSTGGGVSRIFPLPSWQERANVPPSANPGAGSGRGVPDVAGNADPATGYEVVIDGETTVIGGTSAVAPLFAALVARINQKLGKPVGYLNPTLYQLPPEVFHDITEGNNDIANRARIYQAGPGWDPCTGLGSPIGIRLLQALLPSASQAQPLEHHHHHH

SEQ ID NO: 33 - protein, 596 aa, synthetic construct
  REGION 1..596: note = Description of Artificial Sequence: Synthetic polypeptide
MMKSSAAKQTVLCLNRYAVVALPLAIASFAAFGASPASTLWAPTDTKAFVTPAQVEARSAAPLLELAAGETAHIVVSLKLRDEAQLKQLAQAVNQPGNAQFGKFLKRRQFLSQFAPTEAQVQAVVAHLRKNGFVNIHVVPNRLLISADGSAGAVKAAFNTPLVRYQLNGKAGYANTAPAQVPQDLGEIVGSVLGLQNVTRAHPMLKVGERSAAKTLAAGTAKGHNPTEFPTIYDASSAPTAANTTVGIITIGGVSQTLQDLQQFTSANGLASVNTQTIQTGSSNGDYSDDQQGQGEWDLDSQSIVGSAGGAVQQLLFYMADQSASGNTGLTQAFNQAVSDNVAKVINVSLGWCEADANADGTLQAEDRIFATAAAQGQTFSVSSGDEGVYECNNRGYPDGSTYSVSWPASSPNVIAVGGTTLYTTSAGAYSNETVWNEGLDSNGKLWATGGGYSVYESKPSWQSVVSGTPGRRLLPDISFDAAQGTGALIYNYGQLQQIGGTSLASPIFVGLWARLQSANSNSLGFPAASFYSAISSTPSLVHDVKSGNNGYGGYGYNAGTGWDYPTGWGSLDIAKLSAYIRSNGFGHLEHHHHHH

SEQ ID NO: 34 - protein, 552 aa, synthetic construct
  REGION 1..552: note = Description of Artificial Sequence: Synthetic polypeptide
MANGKSTSPASQWVPLPGSNRQLLPQSVPIGPADLKATVALTVKVRSRGKLAELDDAVKKESAKPLKERTYISREELAQRYGADADDLDKVELYANKHHLRVADRDEATRRVVLKGTLEDALSAFHADVHMYQHASGPYRGRRGEILVPAELKDVVTGIFGFDTHPKHRAPRRLMGTSSGTATNLGEFASEFATRYQFPTSSSSTKLDGTGQCIALIELGGGYSNNDLKIFFSEAGVPMPKVVAVSIDHGANHPTPQGLADGEVMLDIEVAGVVAPGAKLAVYFAPNSDSGFQDAIRAAVHDGARKPSVVSISWGEPDDFLTAQSVQSYHEIFTEAAALGVTVCAASGDHGVADLDALHWDKRIHVNHPSSDPLVLCCGGTQIDKNVDVVWNDGTPFDPQVFGGGGWASGGGISPVFGVPDYQKGLPMPSSLSTSQPGRGCPDIAMTADNYRTRVHGVDGPSGGTSAVTPLMACLVARLNQAFEKNLGFVNPLLYANAQAFTDITQGTNGINQTIEGYPAGKGWDACTGLGAPIGTVLLQALGKLEHHHHHH

SEQ ID NO: 35 - protein, 521 aa, synthetic construct
  REGION 1..521: note = Description of Artificial Sequence: Synthetic polypeptide
MKTSNKVALAGSYKKAHSGETTAKINRNTFIEVTLRIRRKKSIESLLNAGKRVDHADYEKEFGASQKDADQVEAFARQYKLSTVEVSLSRRSVILRGSIANMEAAFDVNLSKAVDSHGDDIRVRKGDIYIPEALKDVVEGVFGLDNRKAARPLFKLLKKADGISPQASVSSSFTPNQLAGIYGFPAGFNGKGQTIAIIELGGGYRTTDLTNYFKKLGIKKPSIKAILVDKGKNNPSNANSADGEVMLDIEVAGAVASGAKIVVYFSPNTDKGFLDAITKAVHDTTHKPSVVSISWGGGEAVWTQQSLNSFNEAFKAAAVLGVTVCAAAGDNGSSDGLTDNSVHVDFPASSPYVLACGGTTLKVKNNVITSETVWHDSNDSATGGGVSNVFPLPDYQKNAGVPAAIGTNFIGRGVPDVAGNADPNTGYNVLVDGQQLVIGGTSAVAPLFAGLIACLNQKSGKWSGFINPTLYAANPSVCRDITVGNNRTATGNAGYDARVGWDPCTGLGVFSKLLEHHHHHH

SEQ ID NO: 36 - protein, 569 aa, synthetic construct
  REGION 1..569: note = Description of Artificial Sequence: Synthetic polypeptide
MAPKTSVPHFTTQSRTVLSGSEKAPVAEARGAKPAPLAARITVSVIVRRKTPLKAAHITGEQRLTRAQFNASHAADPAAVKLVQGFAKEFGLTVDPGTPAPGRRTMKLTGTVANMQRAFGVSLAHKTMDGVTYRVREGSINLPAELQGYVVAVLGLDNRPQAEPHFRILGEQGAVAAQAAQGQGFAGPHAGGSTSYTPVQVGELYQFPRGSSASNQTIGIIELGGGFRQTDIAAYFKTLGQKPPQVIAVPIGNGKNNPTNSNSADGEVMLDIEVAGAVAPGARIVVYFAPNTDQGFVDAIAHAIHDTTYKPSVISISWGSAEVNWTVQAMAALDAACQSAAALGITITAASGDNGSSDAVADGENHVDFPASSPHVLACGGTNLQGSGSTISAETVWNAQPQGGATGGGVSNIFPLPTWQASSKVPKPTHPSGGRGVPDVAGDADPASGYVVRVDGQTFVIGGTSAVAPLWAGLIAVANQQNGKSAGFIQPAIYAGQGKPAFRDTVQGSNGSFAAGAGWDACTGLGSPIALQLINAIKPASSKSKSKAIAAKRKTIIRTKKLEHHHHHH

SEQ ID NO: 37 - protein, 8 aa, synthetic construct
  REGION 1..8: note = Description of Artificial Sequence: Synthetic peptide
LEHHHHHH

SEQ ID NO: 38 - protein, 16 aa, synthetic construct
  REGION 1..16: note = Description of Artificial Sequence: Synthetic peptide
EFSWGAAGDDDGGTSA

SEQ ID NO: 39 - protein, 16 aa, synthetic construct
  REGION 1..16: note = Description of Artificial Sequence: Synthetic peptide
EFSWGASGDDCGGTSA

SEQ ID NO: 40 - protein, 16 aa, synthetic construct
  REGION 1..16: note = Description of Artificial Sequence: Synthetic peptide
EFSWGASGDSDGGTSA

SEQ ID NO: 41 - protein, 16 aa, synthetic construct
  REGION 1..16: note = Description of Artificial Sequence: Synthetic peptide
ELSFGSSGDASGGTSL

SEQ ID NO: 42 - protein, 16 aa, synthetic construct
  REGION 1..16: note = Description of Artificial Sequence: Synthetic peptide
EFSWGAAGDSDGGTSA

SEQ ID NO: 43 - protein, 16 aa, synthetic construct
  REGION 1..16: note = Description of Artificial Sequence: Synthetic peptide
ELSLGSSGDESGGTSL

SEQ ID NO: 44 - protein, 16 aa, synthetic construct
  REGION 1..16: note = Description of Artificial Sequence: Synthetic peptide
EFSWGASGDHNGGTSA

SEQ ID NO: 45 - protein, 16 aa, synthetic construct
  REGION 1..16: note = Description of Artificial Sequence: Synthetic peptide
EFSWGAAGDNDGGTSA

SEQ ID NO: 46 - protein, 16 aa, synthetic construct
  REGION 1..16: note = Description of Artificial Sequence: Synthetic peptide
EFSWGASGDNDGGTSA

(19) United States
(12) Patent Application Publication
(10) Pub. No.: US 2024/0315277 A1
(43) Pub. Date: Sep. 26, 2024

(54) USE OF PROTEOLYTIC ENZYMES TO ENHANCE PROTEIN BIOAVAILABILITY

(71) Applicant: The Regents of the University of California, Oakland, CA (US)

(72) Inventors: Justin SIEGEL, Davis, CA (US); Wai Shun MAK, Sacramento, CA (US); John Bruce GERMAN, Davis, CA (US)

(21) Appl. No.: 18/383,335

(22) Filed: Oct. 24, 2023

Related U.S. Application Data
(63) Continuation of application No. 16/767,535, filed on May 27, 2020, now abandoned.
(60) Provisional application No. 62/750,985, filed on Oct. 26, 2018.

(51) Int. Cl. A23J 3/04; A23L 33/185; A61K 38/48

(52) U.S. Cl. CPC A23J 3/04; A23L 33/185; A61K 38/48; C12Y 304/00

(57) ABSTRACT

The present disclosure provides food supplements comprising proteases that can digest a variety of food proteins to enhance their protein bioavailability in the gut.

### CROSS-REFERENCE TO RELATED APPLICATIONS

This application is a continuation of U.S. application Ser. No. 16/767,535, filed May 27, 2020, which is a US National Phase Application Under 371 of PCT/US2019/058173, filed Oct. 25, 2019, which claims priority to U.S. Provisional Application No. 62/750,985, filed Oct. 26, 2018, the disclosures of which are hereby incorporated by reference in their entireties for all purposes.

### REFERENCE TO SUBMISSION OF A SEQUENCE LISTING AS A TEXT FILE

The Sequence Listing written in file 081906-1193828-230520US_SL.txt created on May 26, 2020, 145,154 bytes, machine format IBM-PC, MS-Windows operating system, is hereby incorporated by reference in its entirety for all purposes.

### FIELD OF THE INVENTION

This disclosure relates to food supplements that enhance protein bioavailability.

### BACKGROUND

Advances in analytical techniques to measure the bioavailability of proteins have enabled us to identify high protein quality foods critical to our diets.¹⁻⁵ One of the most important determinants of protein bioavailability lies in their digestibility within the digestive systems where they are processed. Broad-spectrum proteases, including pepsin, trypsin, amino- and carboxy-peptidases, work together to digest food proteins into small peptides, typically 2-4 amino acids long, for absorption in gastrointestinal tract.⁶ However, not all food proteins from our diets are digested/absorbed and some of them are also known to be resistant to proteolytic digest in the gut, thereby limiting the nutritional values.⁷⁻⁹ In addition, this problem is not limited to foods known to be resistant to proteolytic digestion. For example, whey protein is known to be highly bioavailable and fast-digesting.¹⁰ However, studies have shown that whey protein hydrolysates possess a higher bioavailability than intact whey when the proteins/peptides are given within diet-relevant concentrations.¹¹ These results suggests that our digestive systems cannot take advantage of all the proteins in our meal even with protein sources of highest quality. Furthermore, another study has shown that administering specific proteolytic enzymes known to be active on whey protein isolate enhances the concentration of postprandial total serum amino acids.¹²

There is a demand for a broad spectrum of proteases to enhance food protein bioavailability in situ. The present disclosure addresses these and other needs.

### BRIEF SUMMARY

The present disclosure provides proteases that can digest a variety of food proteins to enhance their protein bioavailability.

The disclosure provides methods of improving the digestion of proteins in a food product by a subject. The methods comprise ingesting with the food product a food supplement comprising one or more proteases having an amino acid sequence at least substantially identical to an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, and SEQ ID NO: 24.

In some embodiments, the proteases comprise an active site sequence at least substantially identical to the active site sequence in a protease having an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, and SEQ ID NO: 24.

In some embodiments, the food product comprises:
a) a legume source protein and the food supplement comprises one or more proteases having an amino acid sequence at least substantially identical to an amino acid sequence selected from the group consisting of (SEQ ID NO: 2), (SEQ ID NO: 4), (SEQ ID NO: 8), (SEQ ID NO: 10), (SEQ ID NO: 12), (SEQ ID NO: 14), (SEQ ID NO: 16), (SEQ ID NO: 18), (SEQ ID NO: 20), (SEQ ID NO: 22), and (SEQ ID NO: 24); or
b) a non-legume plant source protein and the food supplement comprises one or more proteases having an amino acid sequence at least substantially identical to an amino acid sequence selected from the group consisting of (SEQ ID NO: 2), (SEQ ID NO: 4), (SEQ ID NO: 8), (SEQ ID NO: 10), (SEQ ID NO: 12), (SEQ ID NO: 14), (SEQ ID NO: 16), (SEQ ID NO: 18), (SEQ ID NO: 22), and (SEQ ID NO: 24); or
c) an animal source protein and the food supplement comprises one or more proteases having an amino acid sequence at least substantially identical to an amino acid sequence selected from the group consisting of (SEQ ID NO: 2), (SEQ ID NO: 4), (SEQ ID NO: 8), (SEQ ID NO: 10), (SEQ ID NO: 12), (SEQ ID NO: 14), (SEQ ID NO: 16), (SEQ ID NO: 18), (SEQ ID NO: 22), and (SEQ ID NO: 24).

In some embodiments, the food product comprises:
a) a legume source protein and the food supplement comprises one or more proteases having an active site sequence at least substantially identical to the active site sequence in a protease having an amino acid sequence selected from the group consisting of (SEQ ID NO: 2), (SEQ ID NO: 4), (SEQ ID NO: 8), (SEQ ID NO: 10), (SEQ ID NO: 12), (SEQ ID NO: 14), (SEQ ID NO: 16), (SEQ ID NO: 18), (SEQ ID NO: 20), (SEQ ID NO: 22), and (SEQ ID NO: 24); or
b) a non-legume plant source protein and the food supplement comprises one or more proteases having an amino acid sequence at least substantially identical to the active site sequence in a protease having an amino acid sequence selected from the group consisting of (SEQ ID NO: 2), (SEQ ID NO: 4), (SEQ ID NO: 8), (SEQ ID NO: 10), (SEQ ID NO: 12), (SEQ ID NO: 14), (SEQ ID NO: 16), (SEQ ID NO: 18), (SEQ ID NO: 22), and (SEQ ID NO: 24); or
c) an animal source protein and the food supplement comprises one or more proteases having an active site sequence at least substantially identical to the active site sequence in a protease having an amino acid sequence selected from the group consisting of (SEQ ID NO: 2), (SEQ ID NO: 4), (SEQ ID NO: 8), (SEQ ID NO: 10), (SEQ ID NO: 12), (SEQ ID NO: 14), (SEQ ID NO: 16), (SEQ ID NO: 18), (SEQ ID NO: 22), and (SEQ ID NO: 24).

In some embodiments, the food product comprises:
a) mung bean protein and the food supplement comprises one or more proteases having an amino acid sequence at least substantially identical to an amino acid sequence selected from the group consisting of SEQ ID NO: 18, SEQ ID NO: 2, SEQ ID NO: 16, and SEQ ID NO: 4; or
b) green bean protein and the food supplement comprises one or more proteases having an amino acid sequence at least substantially identical to an amino acid sequence selected from the group consisting of SEQ ID NO: 18, SEQ ID NO: 12, SEQ ID NO: 16, and SEQ ID NO: 4; or
c) kidney bean protein and the food supplement comprises one or more proteases having an amino acid sequence at least substantially identical to an amino acid sequence selected from the group consisting of SEQ ID NO: 18, SEQ ID NO: 12, SEQ ID NO: 8, SEQ ID NO: 16, SEQ ID NO: 4, and SEQ ID NO: 10; or
d) pea, broccoli, kamut, or asparagus protein and the food supplement comprises one or more proteases having an amino acid sequence selected at least substantially identical to an amino acid sequence from the group consisting of SEQ ID NO: 18, SEQ ID NO: 12, SEQ ID NO: 22, SEQ ID NO: 14, SEQ ID NO: 8, SEQ ID NO: 2, SEQ ID NO: 16, SEQ ID NO: 24, SEQ ID NO: 4, and SEQ ID NO: 10; or
e) pinto bean and lentil bean protein and the food supplement comprises one or more proteases having an amino acid sequence at least substantially identical to an amino acid sequence selected from the group consisting of SEQ ID NO: 18, SEQ ID NO: 12, SEQ ID NO: 16, and SEQ ID NO: 4; or
f) black bean, field pea, cow pea, adzuki bean, lady cream pea, navy pea, black-eyed pea, cranberry bean, yogurt, chlorella, or pistachio protein and the food supplement comprises a protease having an amino acid sequence at least substantially identical to the amino acid sequence of SEQ ID NO: 18; or
g) chick pea protein and the food supplement comprises one or more proteases having an amino acid sequence selected at least substantially identical to an amino acid sequence from the group consisting of SEQ ID NO: 18, SEQ ID NO: 12, SEQ ID NO: 22, SEQ ID NO: 8, SEQ ID NO: 2, SEQ ID NO: 16, SEQ ID NO: 24, SEQ ID NO: 4, SEQ ID NO: 20, and SEQ ID NO: 10; or
h) lupine bean protein and the food supplement comprises one or more proteases having an amino acid sequence selected at least substantially identical to an amino acid sequence from the group consisting of SEQ ID NO: 22, SEQ ID NO: 2, SEQ ID NO: 16, SEQ ID NO: 24, and SEQ ID NO: 4; or
i) baby lima bean protein and the food supplement comprises one or more proteases having an amino acid sequence selected at least substantially identical to an amino acid sequence from the group consisting of SEQ ID NO: 18, SEQ ID NO: 12, SEQ ID NO: 14, SEQ ID NO: 8, SEQ ID NO: 2, SEQ ID NO: 16, SEQ ID NO: 4, and SEQ ID NO: 10; or
j) crowder pea protein and the food supplement comprises one or more proteases having an amino acid sequence selected at least substantially identical to an amino acid sequence from the group consisting of SEQ ID NO: 18, SEQ ID NO: 22, and SEQ ID NO: 24; or
k) pink bean protein and the food supplement comprises one or more proteases having an amino acid sequence selected at least substantially identical to an amino acid sequence from the group consisting of SEQ ID NO: 18, SEQ ID NO: 12, SEQ ID NO: 2, and SEQ ID NO: 4; or
l) cannellini bean protein and the food supplement comprises one or more proteases having an amino acid sequence selected at least substantially identical to an amino acid sequence from the group consisting of SEQ ID NO: 18, SEQ ID NO: 16, and SEQ ID NO: 4; or
m) pigeon pea, yellow split pea, white bean, pork, pea protein powder, buckwheat, barley, or turkey protein and the food supplement comprises one or more proteases having an amino acid sequence selected at least substantially identical to an amino acid sequence from the group consisting of SEQ ID NO: 18, SEQ ID NO: 12, SEQ ID NO: 14, SEQ ID NO: 8, SEQ ID NO: 2, SEQ ID NO: 16, SEQ ID NO: 4, and SEQ ID NO: 10; or
n) Indian red lentil bean, whey, peanut, cashew, or chicken egg protein and the food supplement comprises one or more proteases having an amino acid sequence at least substantially identical to an amino acid sequence selected from the group consisting of SEQ ID NO: 18, and SEQ ID NO: 4; or
o) great northern bean, hemp protein powder, almond, or beef protein and the food supplement comprises one or more proteases having an amino acid sequence at least substantially identical to an amino acid sequence selected from the group consisting of SEQ ID NO: 18, SEQ ID NO: 16, and SEQ ID NO: 4; or
p) fava bean or salmon protein and the food supplement comprises one or more proteases having an amino acid sequence at least substantially identical to an amino acid sequence selected from the group consisting of SEQ ID NO: 18, SEQ ID NO: 2, SEQ ID NO: 16, and SEQ ID NO: 4; or
q) chicken protein and the food supplement comprises one or more proteases having an amino acid sequence at least substantially identical to an amino acid sequence selected from the group consisting of SEQ ID NO: 18, SEQ ID NO: 8, SEQ ID NO: 2, SEQ ID NO: 16, and SEQ ID NO: 4; or
r) flounder protein and the food supplement comprises one or more proteases having an amino acid sequence at least substantially identical to an amino acid sequence selected from the group consisting of SEQ ID NO: 18, SEQ ID NO: 22, SEQ ID NO: 16, and SEQ ID NO: 4; or
s) casein and the food supplement comprises one or more proteases having an amino acid sequence selected at least substantially identical to an amino acid sequence from the group consisting of SEQ ID NO: 18, SEQ ID NO: 12, SEQ ID NO: 22, SEQ ID NO: 14, SEQ ID NO: 8, SEQ ID NO: 16, and SEQ ID NO: 10; or
t) quinoa protein and the food supplement comprises one or more proteases having an amino acid sequence at least substantially identical to an amino acid sequence selected from the group consisting of SEQ ID NO: 16, and SEQ ID NO: 4; or
u) chia seed protein and the food supplement comprises one or more proteases having an amino acid sequence selected at least substantially identical to an amino acid sequence from the group consisting of SEQ ID NO: 18, SEQ ID NO: 12, SEQ ID NO: 22, SEQ ID NO: 16, SEQ ID NO: 4, and SEQ ID NO: 10; or
v) soy bean protein and the food supplement comprises one or more proteases having an amino acid sequence at least substantially identical to an amino acid sequence selected from the group consisting of SEQ ID NO: 18, SEQ ID NO: 12, SEQ ID NO: 22, SEQ ID NO: 14, SEQ ID NO: 8, SEQ ID NO: 2, SEQ ID NO: 16, SEQ ID NO: 24, SEQ ID NO: 4, SEQ ID NO: 20, and SEQ ID NO: 10; or
w) rye berry protein and the food supplement comprises one or more proteases having an amino acid sequence at least substantially identical to an amino acid sequence selected from the group consisting of SEQ ID NO: 18, SEQ ID NO: 22, SEQ ID NO: 2, and SEQ ID NO: 4; or
x) amaranth protein and the food supplement comprises one or more proteases having an amino acid sequence at least substantially identical to an amino acid sequence selected from the group consisting of SEQ ID NO: 18, SEQ ID NO: 2, SEQ ID NO: 16, and SEQ ID NO: 4; or
y) spirulina protein and the food supplement comprises one or more proteases having an amino acid sequence at least substantially identical to an amino acid sequence selected from the group consisting of SEQ ID NO: 18, SEQ ID NO: 12, SEQ ID NO: 14, SEQ ID NO: 8, SEQ ID NO: 2, SEQ ID NO: 16, SEQ ID NO: 4, and SEQ ID NO: 10; or
z) sunflower seed protein and the food supplement comprises a protease having an amino acid sequence at least substantially identical to the amino acid sequence of SEQ ID NO: 4.

The food supplement may be ingested simultaneously with the food product, or just before or just after ingestion. In some embodiments, the food supplement is incorporated into the food product.

The disclosure also provides a food supplement or food product comprising one or more proteases of the disclosure and optionally one or more food proteins disclosed here. The food supplement or food product may further comprise one or more of a bulking agent, a carrier, a sweetener, a coating, a preservative, a binding agent, a desiccant, a lubricating agent, a filler, a solubilizing agent, an emulsifier, a stabilizer, or a matrix modifier.

The food supplement may be in the form of a tablet, capsule, powder, granule, pellet, soft gel, hard gel, controlled release form, liquid, syrup, suspension, or emulsion.

The disclosure also provides methods of making the food supplement of the disclosure. The methods comprising mixing one or more proteases of the disclosure with one or more of a bulking agent, a carrier, a sweetener, a coating, a preservative, a binding agent, a desiccant, a lubricating agent, a filler, a solubilizing agent, an emulsifier, a stabilizer, or a matrix modifier. In some embodiments, the proteases are recombinantly produced, for example using E. coli. The proteases of the disclosure can be recombinantly produced using an expression cassette comprising a nucleic acid sequence at least substantially identical to an open reading from SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, or SEQ ID NO: 23.

### Definitions

The terms “identical” or percent “identity,” in the context of two or more nucleic acids or polypeptide sequences, (e.g., two proteases of the disclosure and polynucleotides that encode them) refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

In the typical embodiment, Promals3D is used for sequence alignment and sequence comparisons. See, e.g., Pei, et al. Nucleic Acids Res. 2008 36(7):2295-2300, which is incorporated herein by reference. Other algorithms that are suitable for determining percent sequence identity and sequence similarity include the BLAST and BLAST 2.0 algorithms, which are described in Altschul et al., J. Mol. Biol. 215:403-410, 1990 and Altschuel et al., Nucleic Acids Res. 25:3389-3402, 1977, respectively. Software for performing BLAST analyses is publicly available through the National Center for Biotechnology Information.

The phrase “substantially identical,” in the context of two polynucleotides or polypeptides of the disclosure, refers to two or more sequences or subsequences that have at least 60%, 65%, 70%, 75%, 80%, 85%, 90%, 95% or 99% nucleotide or amino acid residue identity, when compared and aligned for maximum correspondence, as measured using one of the above sequence comparison algorithms or by visual inspection. In the typical embodiment, the sequences are at least about 80% identical, usually at least about 90% identical, and often at least 95% identical. Substantial identity can be determined over a subsequence in a given polynucleotide or polypeptide (e.g., in the case of SSEs) or over the entire length of the molecule.

“Operably linked” indicates that two or more DNA segments are joined together such that they function in concert for their intended purposes. For example, coding sequences are operably linked to promoter in the correct reading frame such that transcription initiates in the promoter and proceeds through the coding segment(s) to the terminator.

A “polynucleotide” is a single- or double-stranded polymer of deoxyribonucleotide or ribonucleotide bases typically read from the 5′ to the 3′ end. Polynucleotides include RNA and DNA, and may be isolated from natural sources, synthesized in vitro, or prepared from a combination of natural and synthetic molecules. When the term is applied to double-stranded molecules it is used to denote overall length and will be understood to be equivalent to the term “base pairs”.

A “polypeptide” or “protein” is a polymer of amino acid residues joined by peptide bonds, whether produced naturally or synthetically. Polypeptides of less than about 75 amino acid residues are also referred to here as peptides or oligopeptides.

The term “promoter” is used herein for its art-recognized meaning to denote a portion of a gene containing DNA sequences that provide for the binding of RNA polymerase and initiation of transcription of an operably linked coding sequence. Promoter sequences are typically found in the 5′ non-coding regions of genes.

### BRIEF DESCRIPTION OF THE DRAWINGS

FIG. 1 is a computer molecular model showing the position of active site residues in the proteases of the disclosure. Structural alignment of protein molecular models was performed using the TM-align algorithm (TMalign.f). See, Y. Zhang & J. Skolnick, Nucleic Acids Research, 33: 2302-2309 (2005); Y. Zhang & J. Skolnick, Proteins, 57: 702-710 (2004); and J. Xu & Y. Zhang, Bioinformatics, 26, 889-895 (2010). The algorithm is also described in Zhang and Skolnick, Nucleic Acids Research, 33(7):2302, 2005. The position numbering refers to the corresponding amino acid positions in the alignment shown in FIG. 2.

FIG. 2 is a sequence alignment which shows active site amino acid identities and similarities shared by the proteases of the disclosure.

FIG. 3 is a heat map on the activities of the 12 proteases tested against 56 food substrates. Light color denotes that the protease degraded the more than 70% of the major protein species in the food source into smaller peptides after a 24-hour incubation with 0.1 mg/ml of the protease at 37° C. Dark color denotes that the protease degrades less than 70% of the major protein species or are inactive on the food proteins tested.

FIG. 4 shows an alignment of the predicted secondary structure elements in the 12 exemplified proteases.

FIG. 5 shows a pairwise comparison of the active site sequences of the 12 exemplified proteases.

### DETAILED DESCRIPTION

The present disclosure provides proteases that can digest a variety of food proteins under acidic conditions of the gut to enhance their protein bioavailability. In particular, the disclosure is based, at least in part, on the discovery of proteases and/or groups of proteases that are particularly active against certain target food proteins or classes of target food proteins. Thus, the present disclosure provides combinations of food proteins and one or more proteases that are selected for the ability to hydrolyse the target food proteins.

### Proteases

The proteases, also referred to as endopeptidases, useful in the present disclosure are enzymes, typically derived from a microbial source, which are capable of hydrolyzing proteins into small peptides, typically 2-4 amino acids long, for absorption in the gastrointestinal tract. Such proteases are active in an acidic pH environment (pH from about 2 to about 6) of the gut. Proteases suitable for use in the present disclosure can be prepared by known methods using publically available sequence information.

The proteases of the disclosure may be defined by their degree of sequence identity to the exemplified proteases (SEQ ID NO: 2, 4, 6, 8, 10, 12, 14, 16, 18, 20, 22, or 24). In the typical embodiment, the amino acid sequences of the proteases of the disclosure are at least substantially identical (as defined above) to the sequence of one or more of the exemplified proteases.

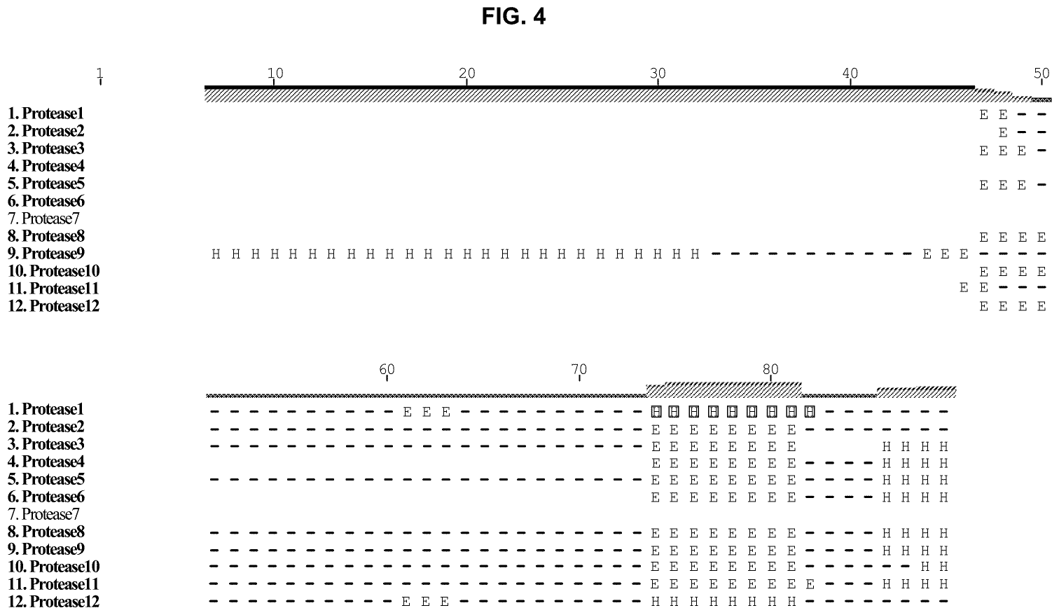

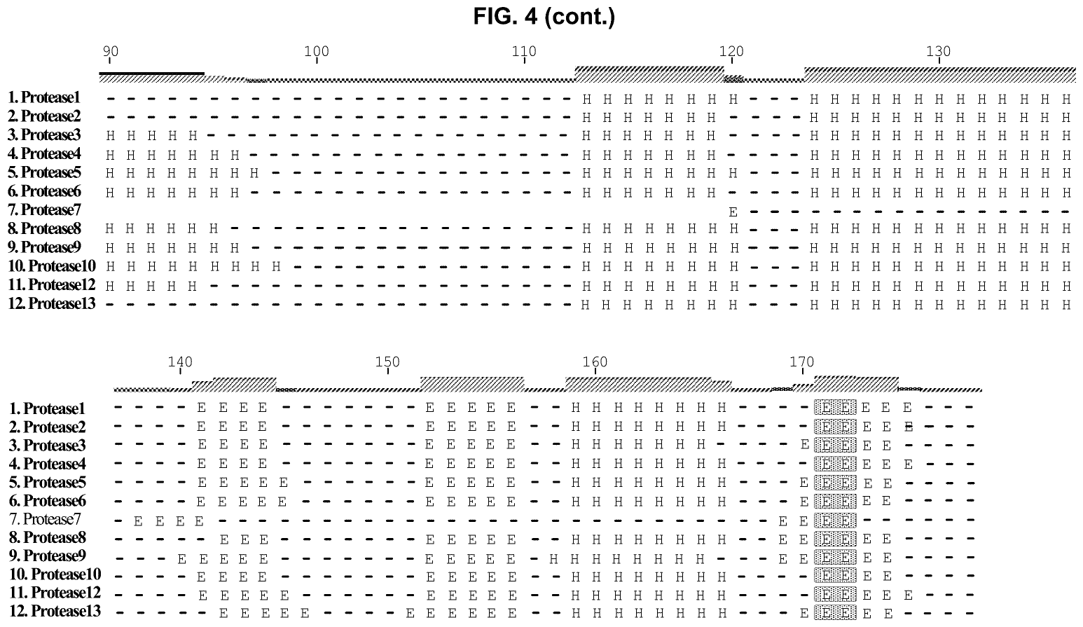

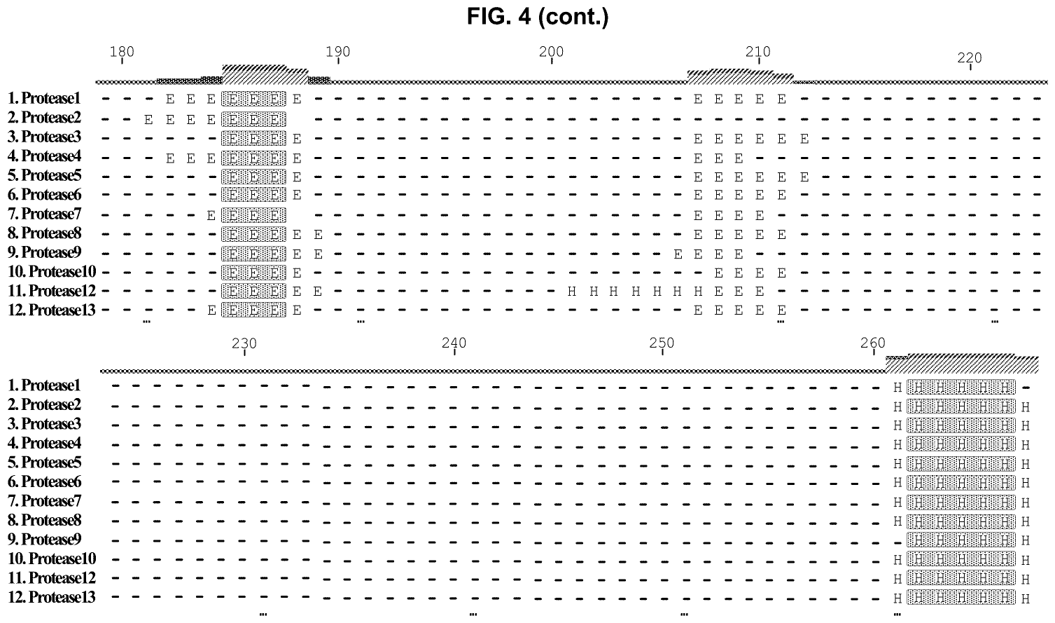

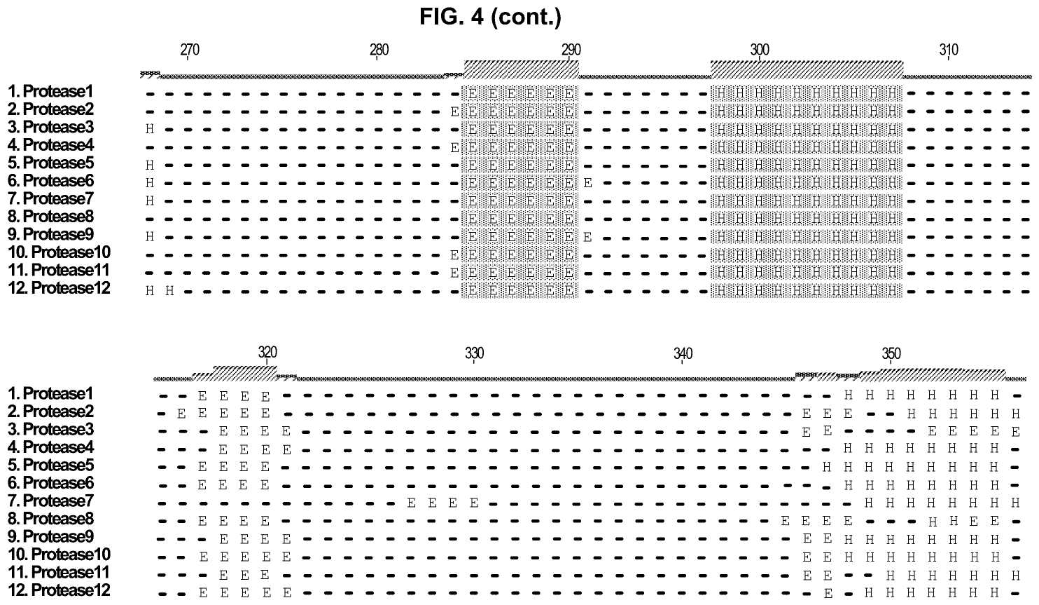

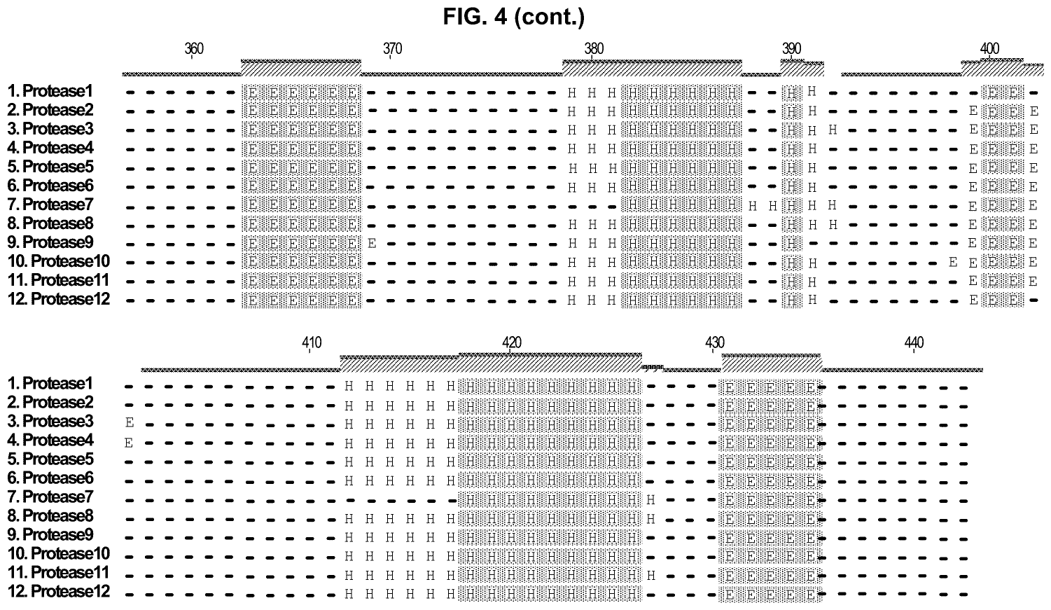

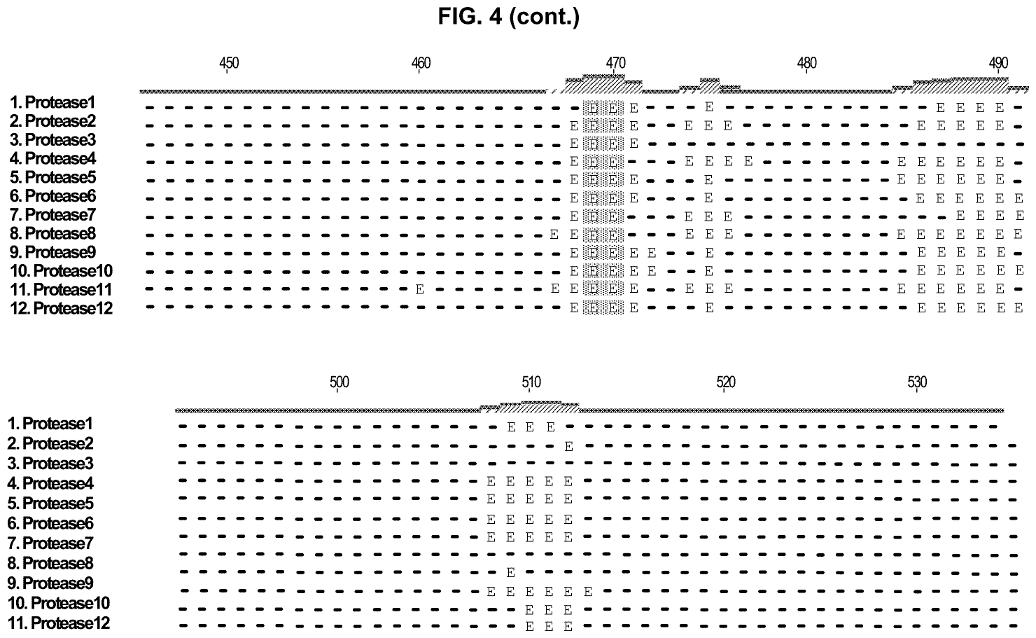

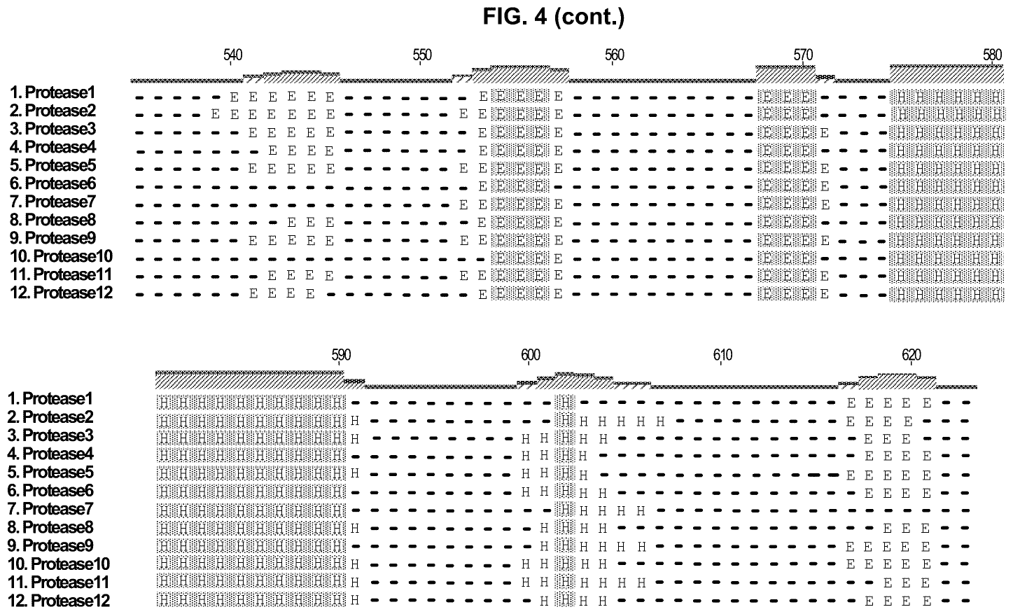

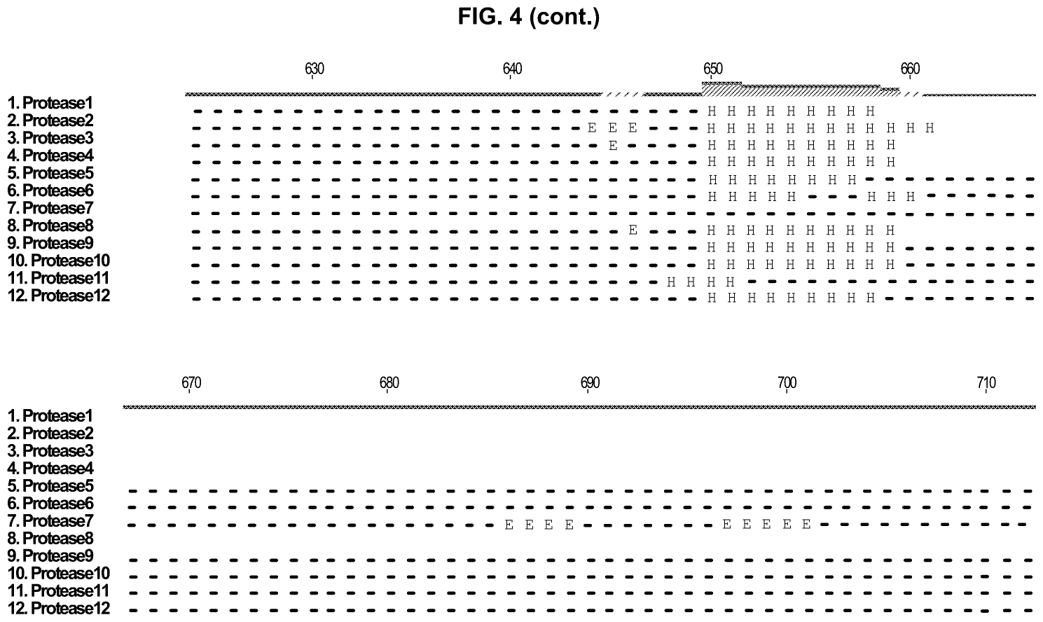

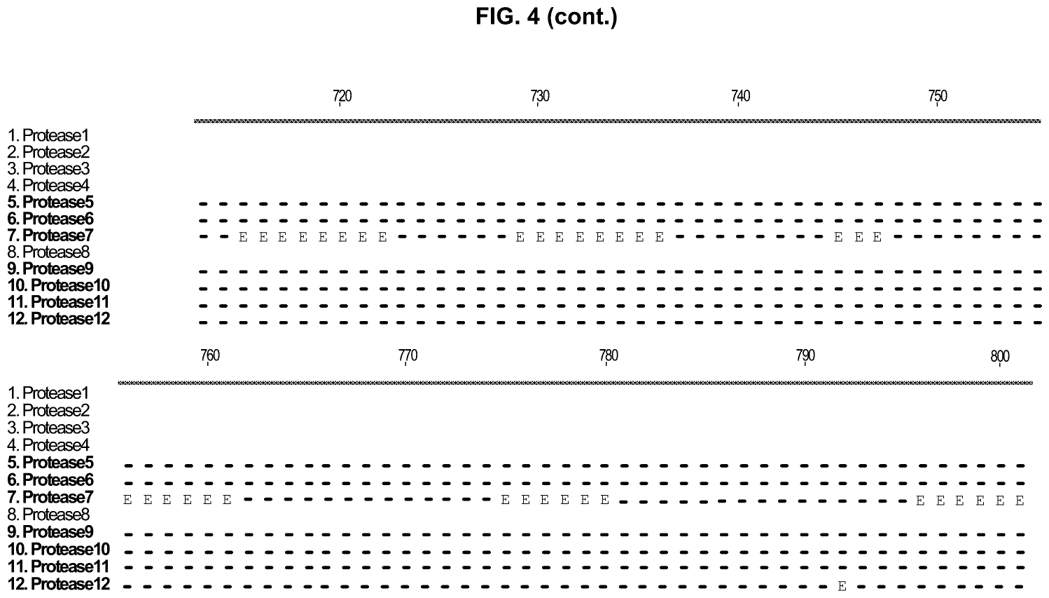

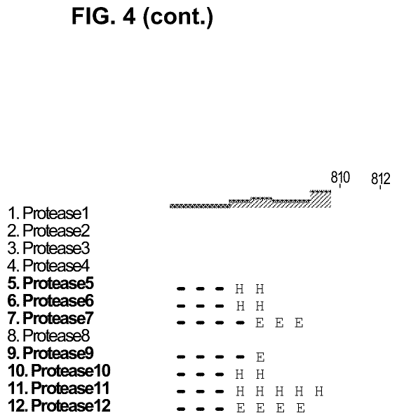

Proteases of the disclosure can also be identified by sequence comparisons that take into account the secondary structure elements (SSEs) in the protein. SSEs can be identified using, for example, Jpre4 (on the internet at compbio.dundee.ac.uk/jpred). The algorithm is also described in Drozdetskiy et al., Nucleic Acids Research, 43:W1, W389-W394, 2015. FIG. 4 shows an alignment of the predicted secondary structure elements in the 12 exemplified proteases. The highlighted residues are the 80 structurally conserved residues that define the protease enzyme scaffold of the exemplified proteases. For example, the following 80 residues make up the SSE sequences of SEQ ID NO: 18 (Protease 9): 163-164 (E), 171-173 (E), 227-231 (H), 245-250 (E), 258-267 (H), 313-318 (E), 332-338 (H), 346-347 (E), 366-374 (H), 379-383 (E), 415-416 (E), 489-491 (E), 496-498 (E), 503-518 (H), 530 (H). (E=beta-sheet, H=alpha-helix).

“SSE sequence identity” is determined by aligning a test protein sequence with a protease of the disclosure (the reference sequence) using the alignment tools described above. The SSE sequence identity is then determined by calculating the percent sequence identity for the test SSE sequences relative to the reference SSE sequences. Usually, the SSE sequences are at least substantially identical (as defined above) to the SSE sequences of one or more of the exemplified proteases.

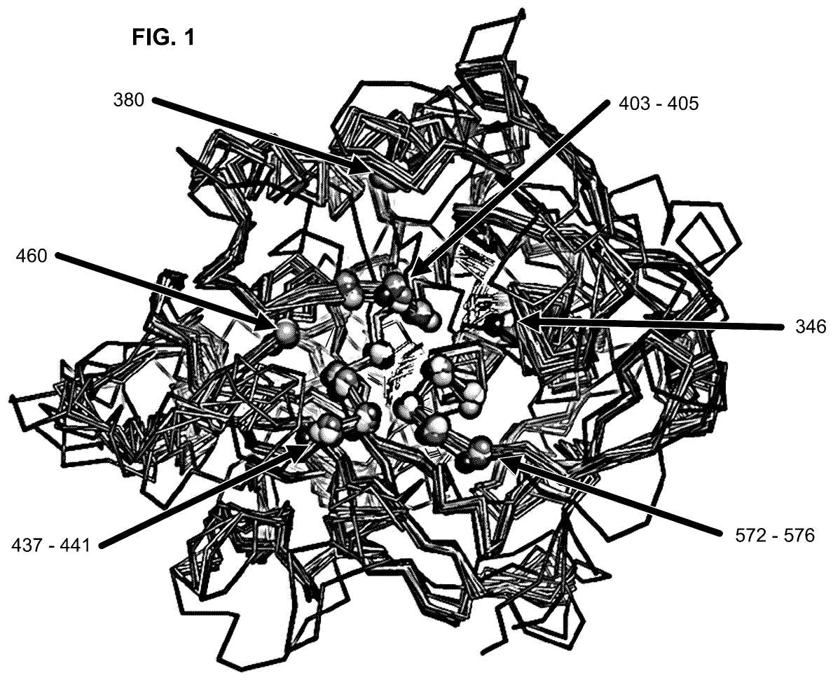

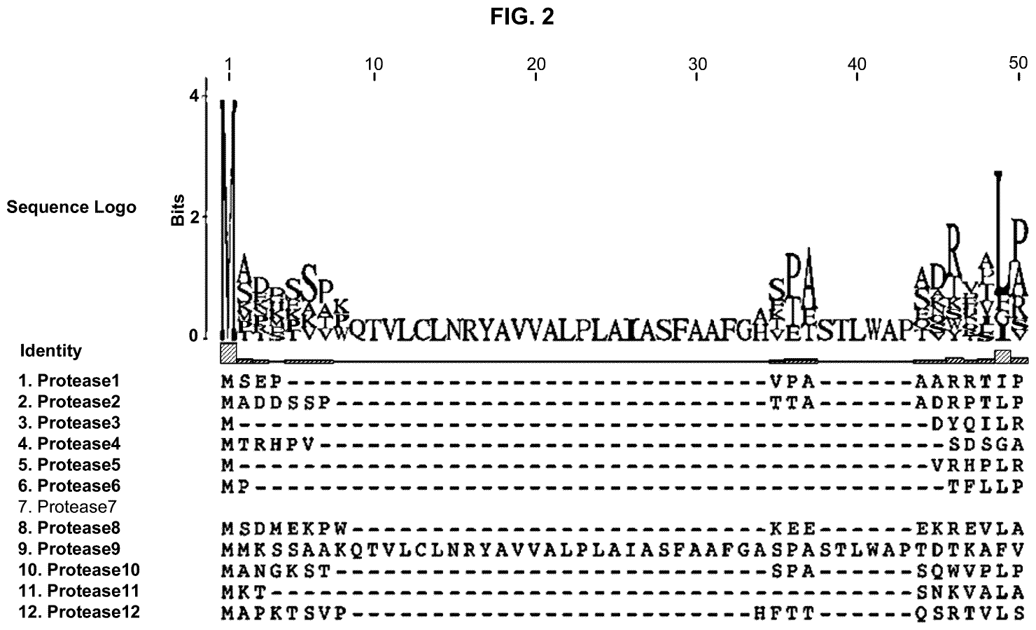

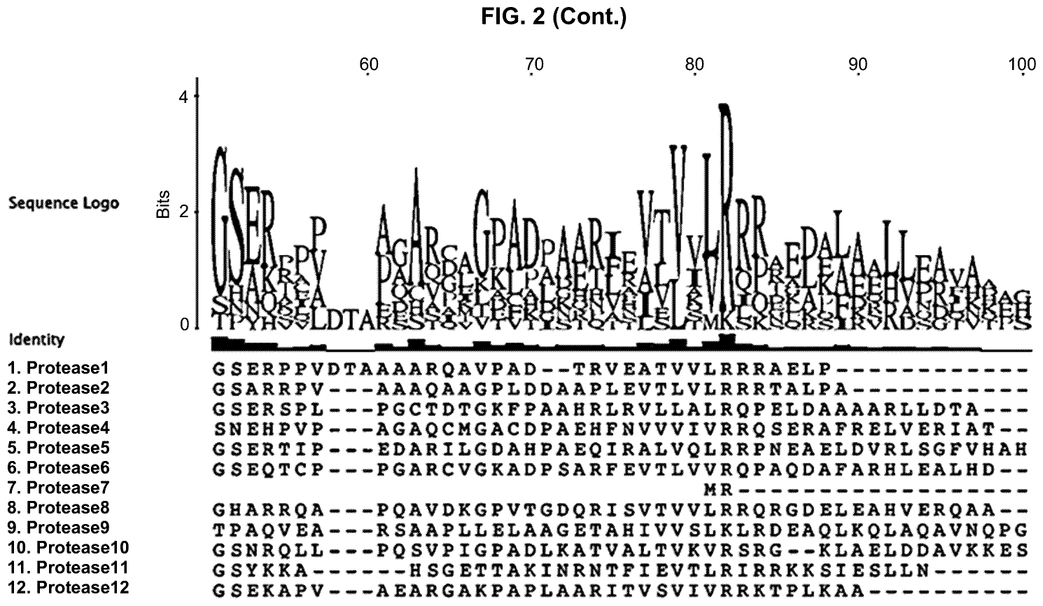

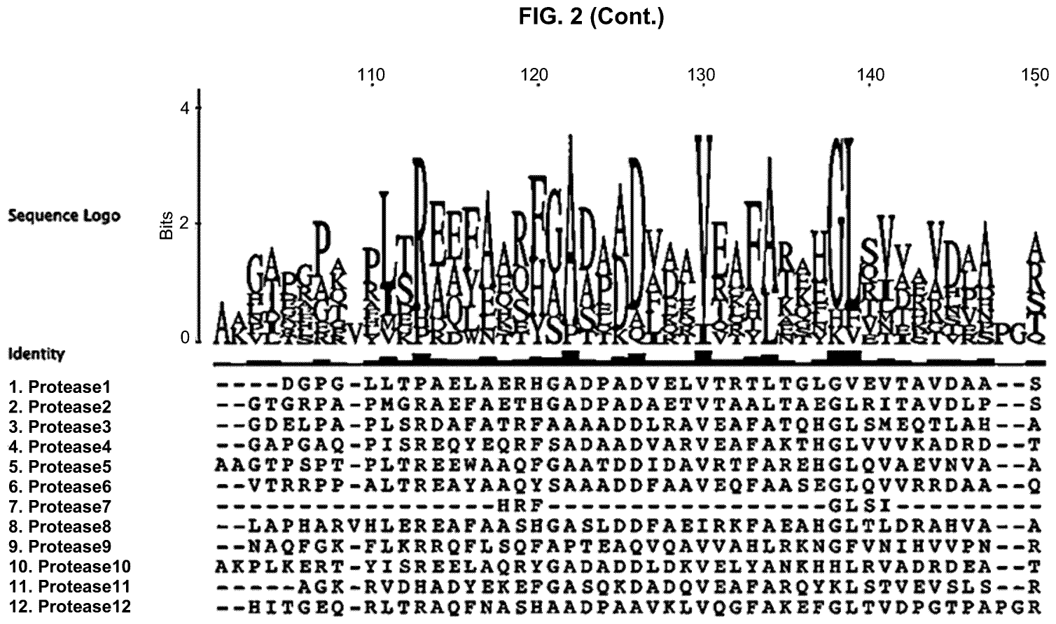

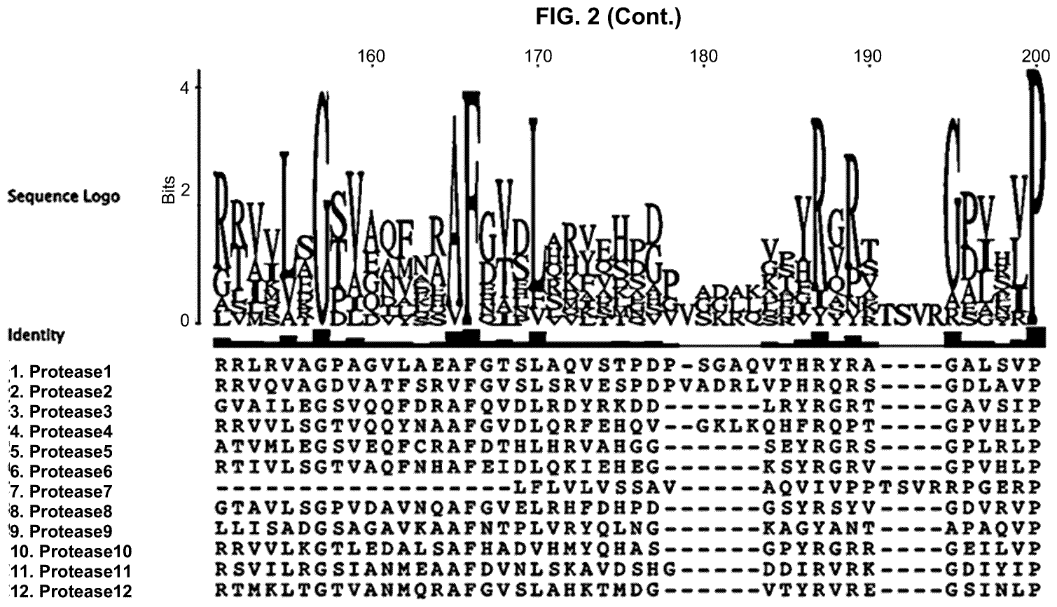

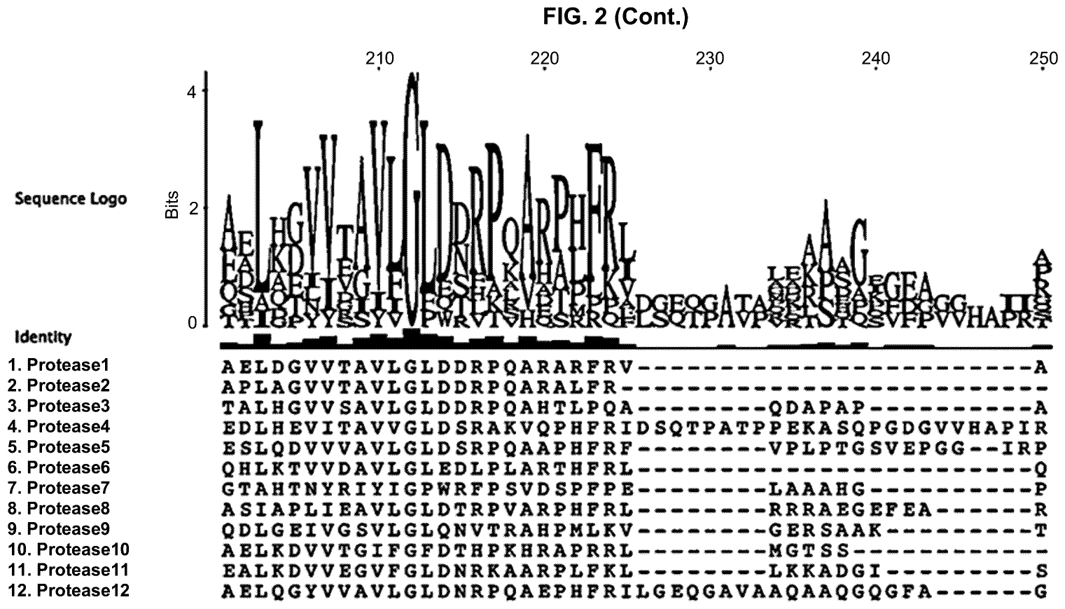

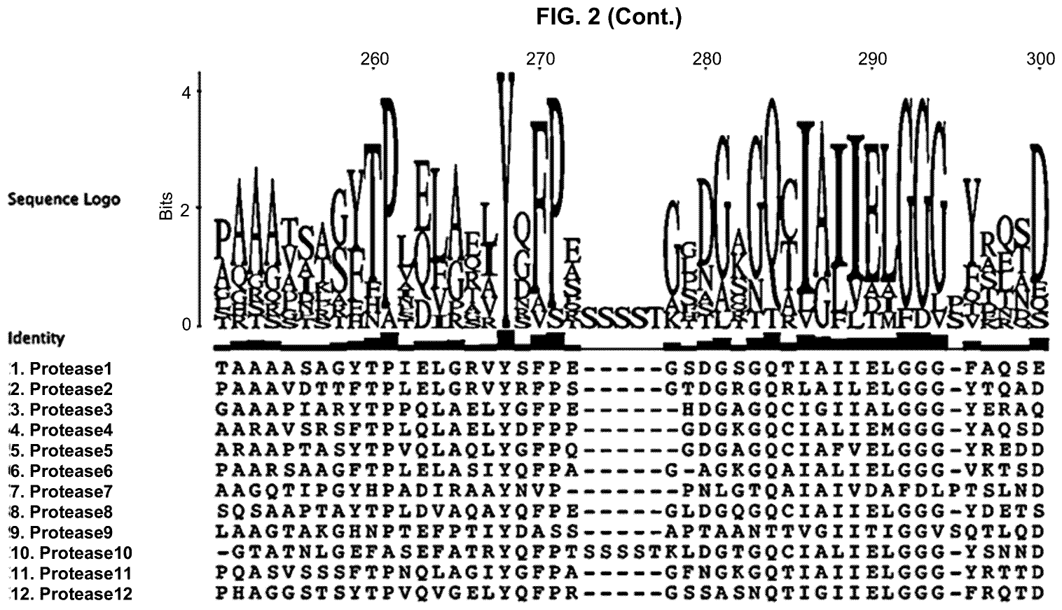

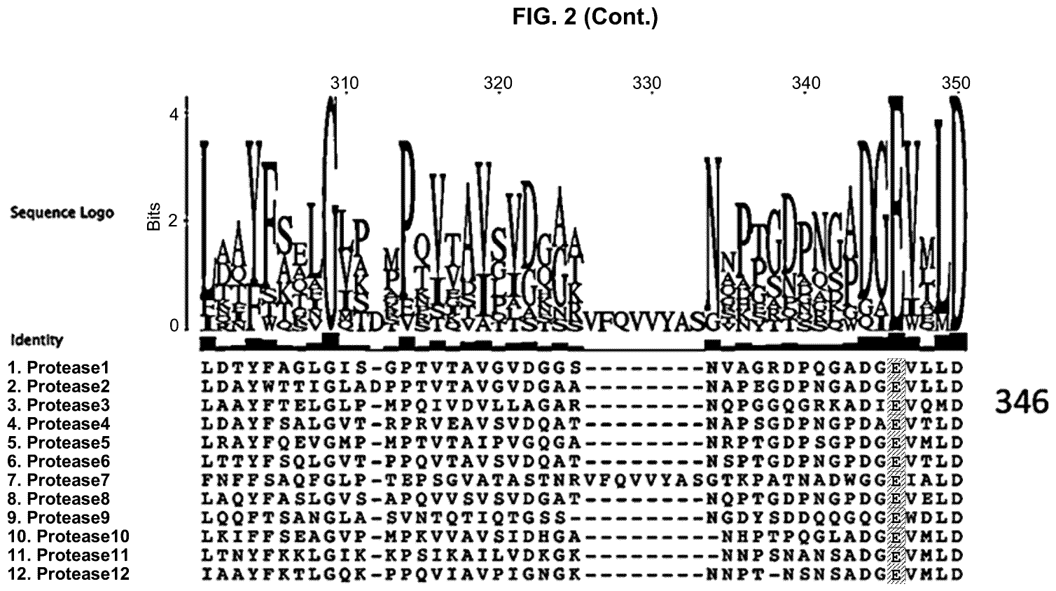

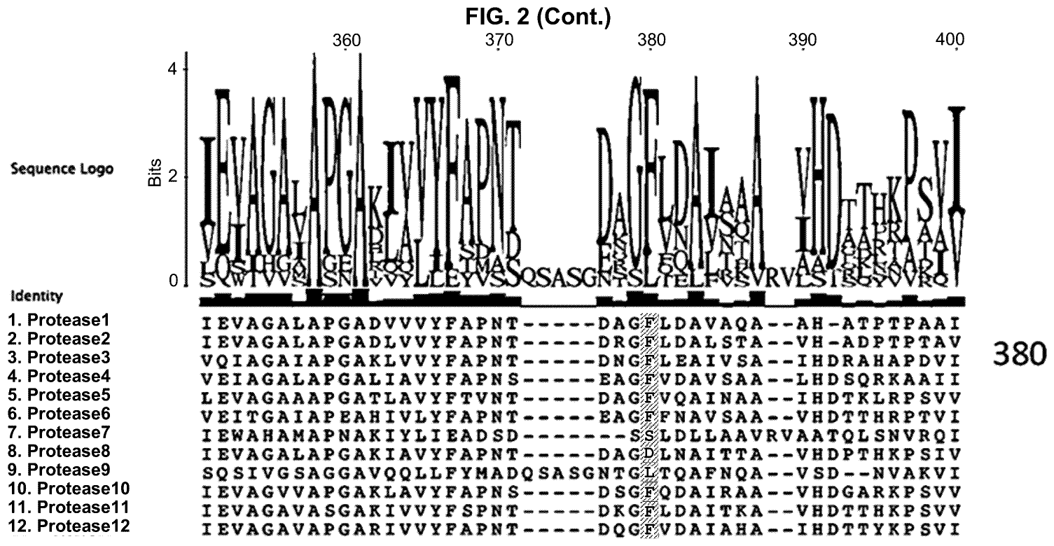

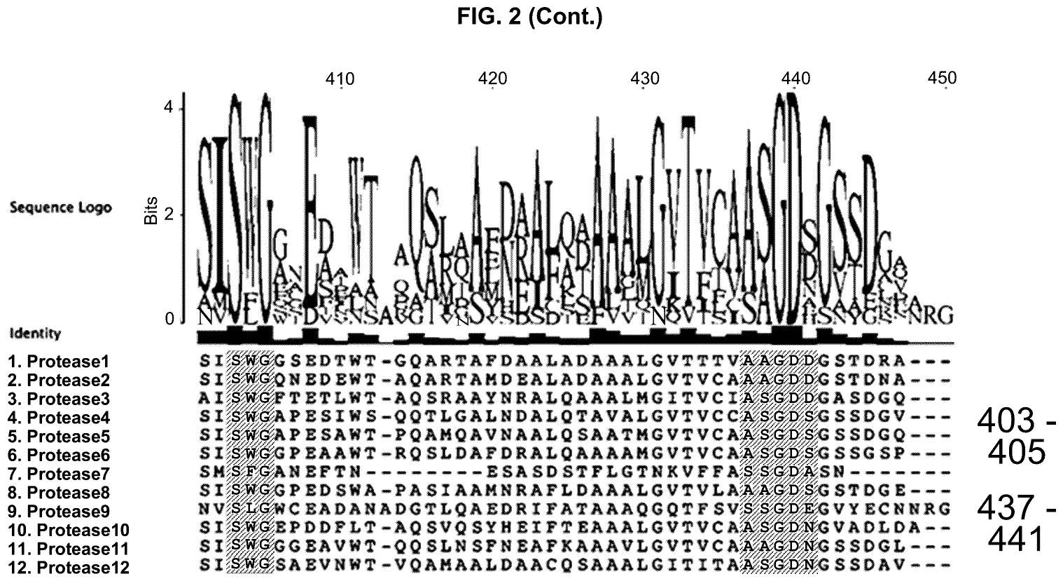

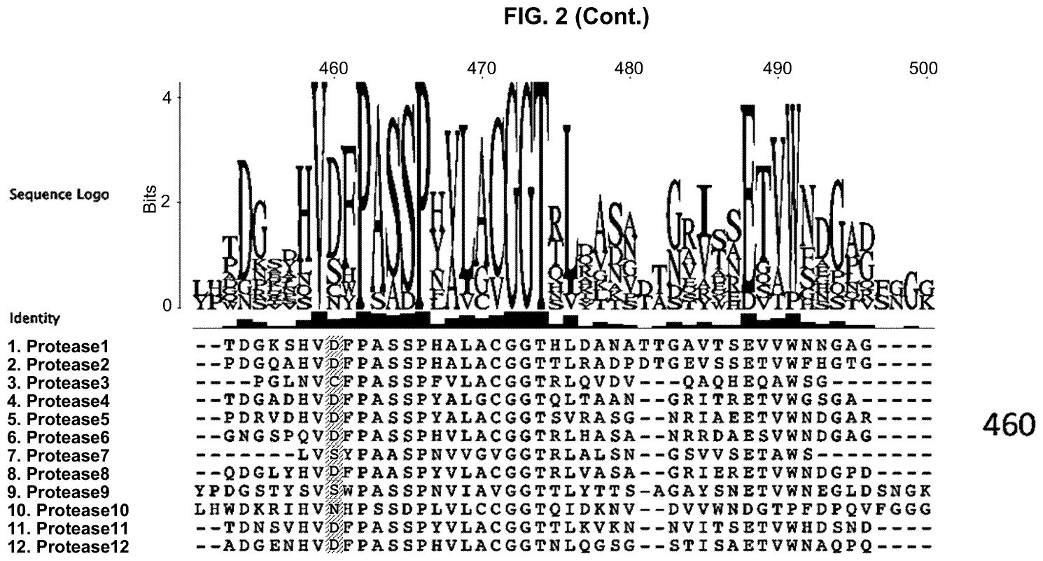

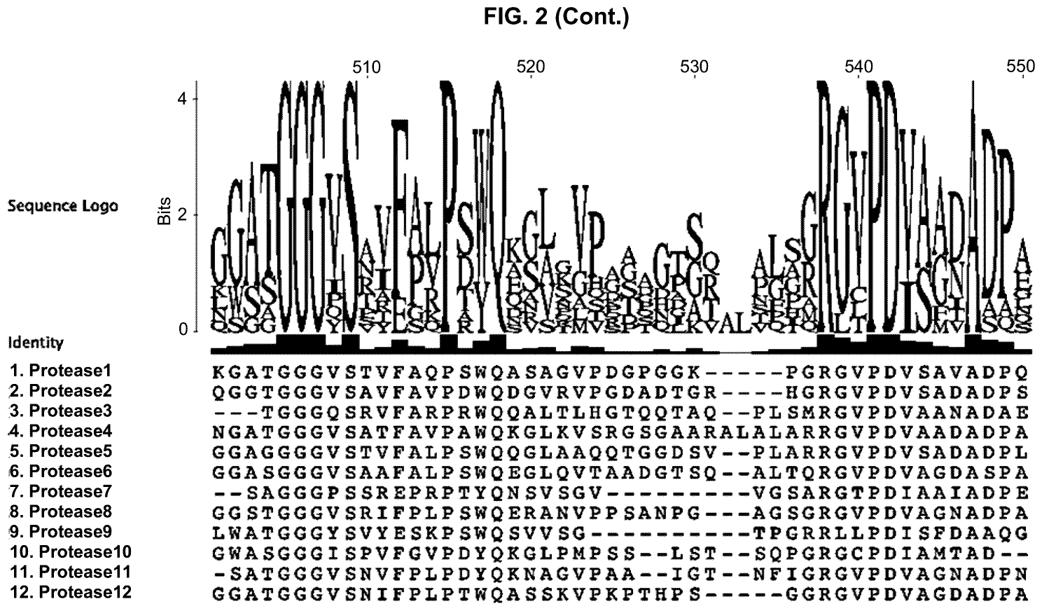

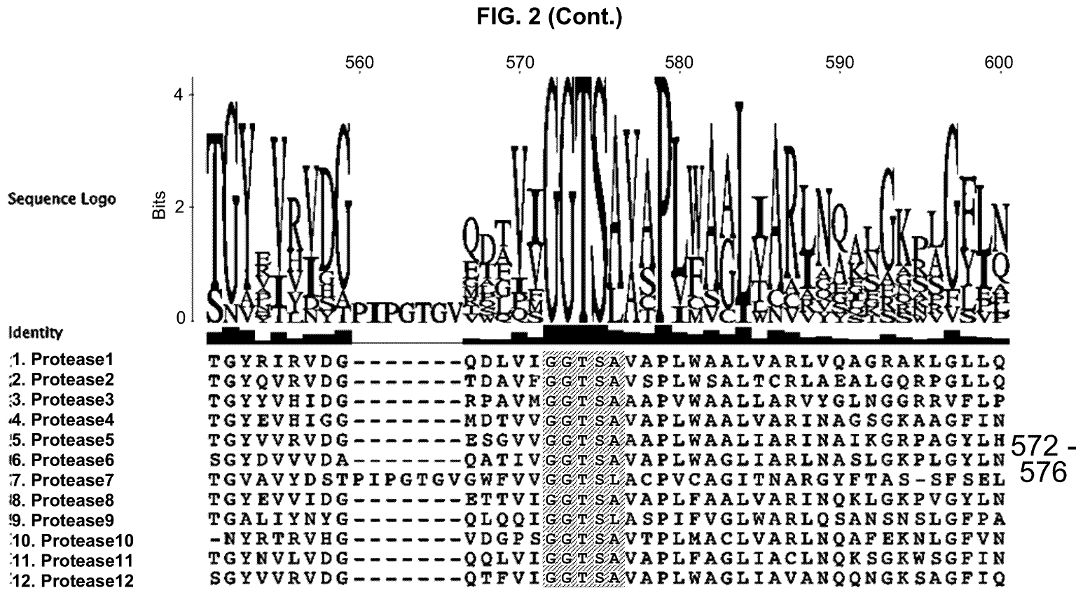

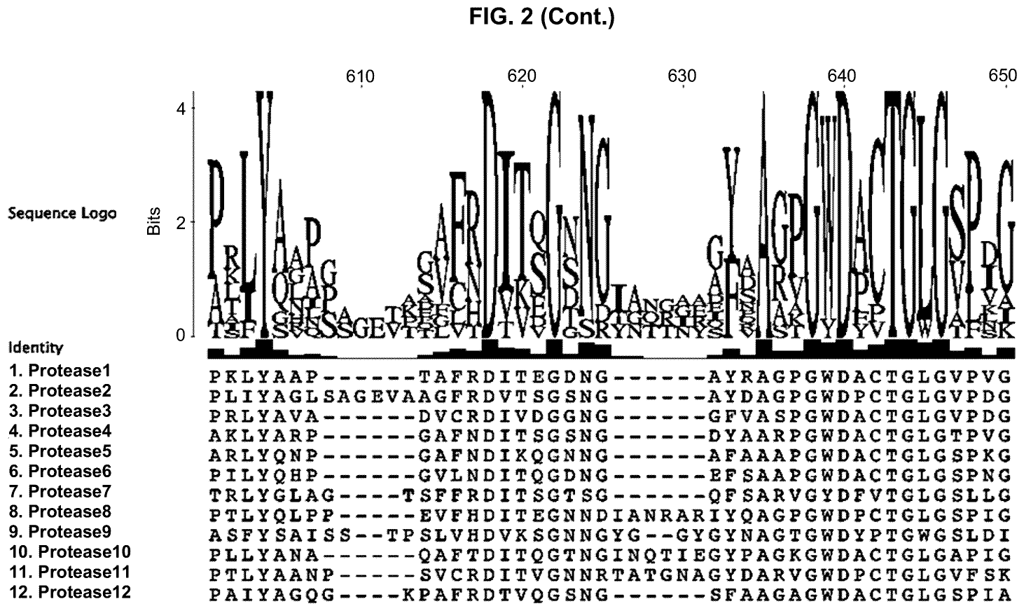

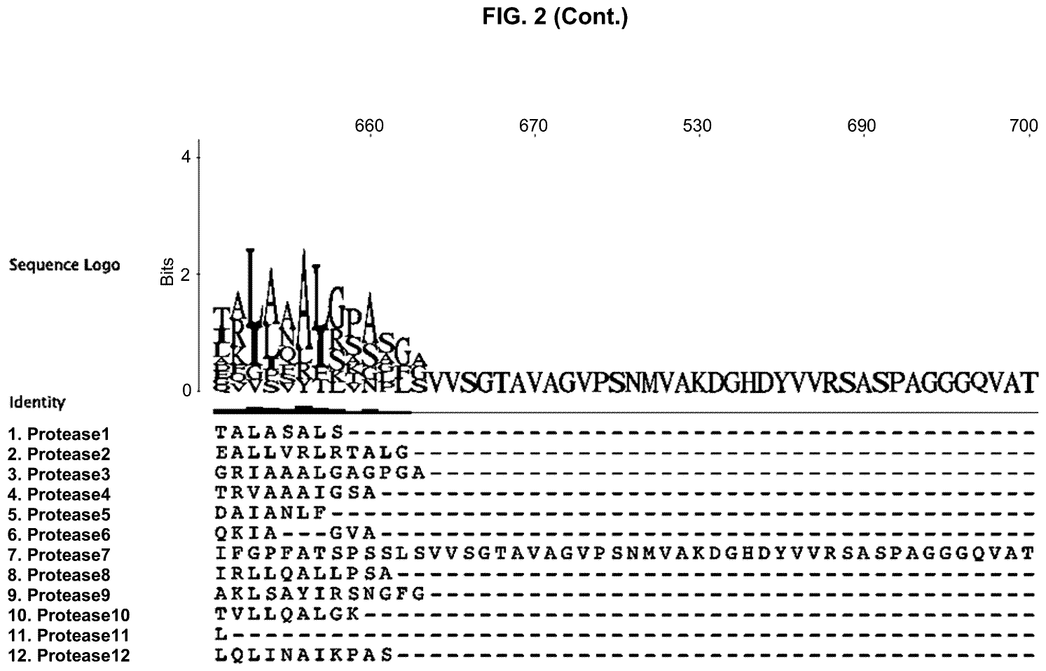

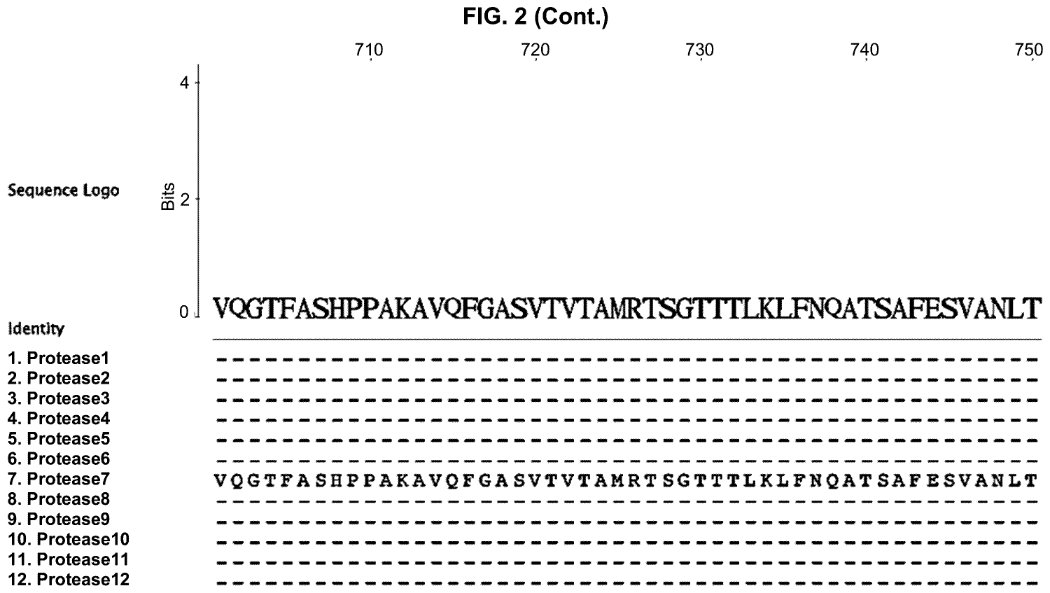

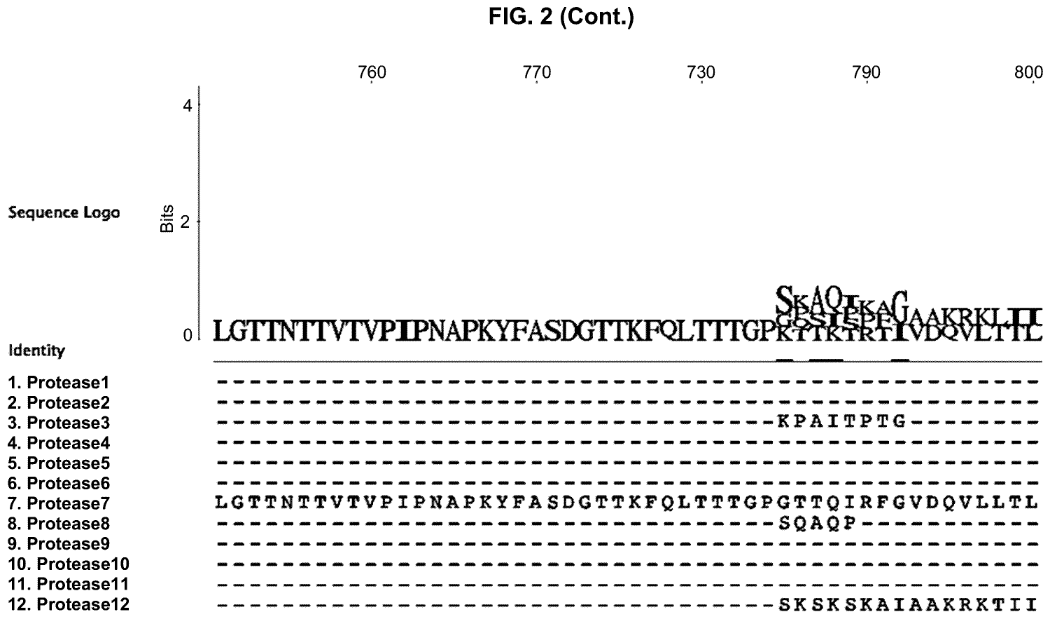

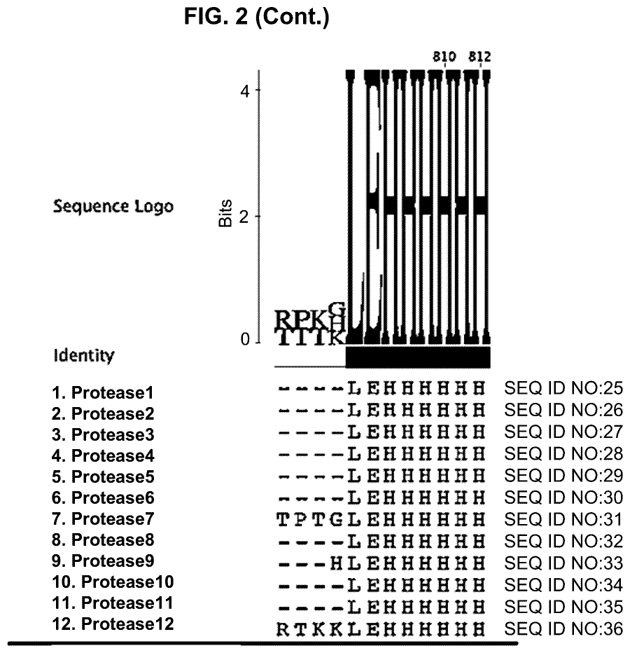

A protease of the disclosure may be further identified by the presence of certain active site residues that align with the active site residues identified in one or more of the exemplified proteases. Active site residues in the exemplified proteases can easily be determined by reference to FIG. 2. In particular, the active site residues of the 12 exemplified proteases are those residues in each protease that correspond to residues 346, 380, 403-405, 437-441, 460, and 572-576 identified in FIGS. 1 and 2. The “active site sequence” of any protease of the disclosure is formed by extracting the amino acids from these positions and concatenating them together. Thus, the active site sequence of each of the 12 exemplified proteases is as follows:

(SEQ ID NO: 38)
Protease 1: EFSWGAAGDDDGGTSA;
(SEQ ID NO: 38)
Protease 2: EFSWGAAGDDDGGTSA;
(SEQ ID NO: 39)
Protease 3: EFSWGASGDDCGGTSA;
(SEQ ID NO: 40)
Protease 4: EFSWGASGDSDGGTSA;
(SEQ ID NO: 40)
Protease 5: EFSWGASGDSDGGTSA;
(SEQ ID NO: 40)
Protease 6: EFSWGASGDSDGGTSA;
(SEQ ID NO: 41)
Protease 7: ELSFGSSGDASGGTSL;
(SEQ ID NO: 42)
Protease 8: EFSWGAAGDSDGGTSA;
(SEQ ID NO: 43)
Protease 9: ELSLGSSGDESGGTSL;
(SEQ ID NO: 44)
Protease 10: EFSWGASGDHNGGTSA;
(SEQ ID NO: 45)
Protease 11: EFSWGAAGDNDGGTSA;
(SEQ ID NO: 46)
Protease 12: EFSWGASGDNDGGTSA.

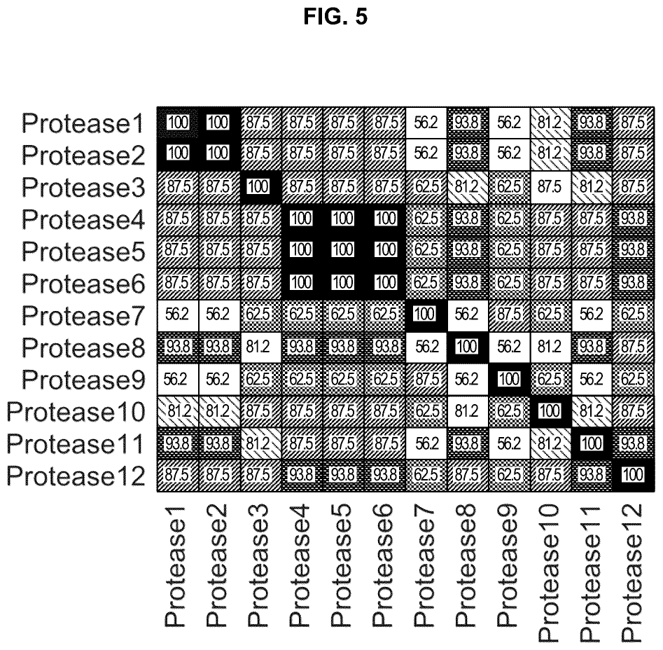

In the typical embodiment, the active site sequences of the proteases of the disclosure are at least substantially identical (as defined above) to the active site sequences of one or more of the exemplified proteases. Thus, for example, a protease of the disclosure can be identified by alignment to SEQ ID NO: 18 (Protease 9) and identifying those residues that align with residues 296, 330, 349, 350, 351, 383, 384, 385, 386, 387, 406, 500, 501, 502, 503, 504 in SEQ ID NO: 18 (the active site sequence). In this example, a protease of the disclosure can be identified as one having an active site sequence at least substantially identical (as described above) to the active site sequence of Protease 9 (SEQ ID NO: 18). A pairwise comparison of the active site sequences of the 12 exemplified proteases is shown in FIG. 5.

In some preferred embodiments of the disclosure, a protease of the disclosure can be identified by both SSE sequence identity and active site sequence identity analyses described above. Thus, a protease of the disclosure can be identified as one having SSE sequences at least substantially identical to the SSE sequences of one or more of the exemplified proteases and an active site sequence at least substantially identical to the active site sequence of one or more of the exemplified proteases.

One of skill will recognize that the proteases of the disclosure may be modified for any of a number of desired properties, such as stability, increased enzymatic activity, and the like. Typically, a modified protease of the disclosure will maintain at least about 90% of the enzymatic activity of the unmodified form, as measured using a standard assay for protease activity. Such assays can also be used to confirm that a protease identified by the sequence and/or structural analyses described above is a protease of the disclosure. A typical assay is performed using sodium dodecyl sulfate-polyacrylamide gel electrophoresis (SDS-PAGE) analysis. The proteolytic activities are determined through monitoring the disappearance of food protein bands on SDS-PAGE gels after an overnight incubation with each protease.¹³⁻¹⁵

The proteases of the disclosure or nucleic acids encoding them are usually derived from microbial sources, such as fungi, bacteria, and the like. Methods for identifying and isolating desired proteins and nucleic acids are well known to those of skill in the art.

The proteases of the disclosure can be made using standard methods well known to those of skill in the art. For example, shorter polypeptides (i.e., oligopeptides) can be made synthetically. For longer polypeptides, recombinant expression can be conveniently used. Recombinant expression in a variety of host cells, including prokaryotic hosts, such as E. coli and eukaryotic cells, such as yeast, is well known in the art. The nucleic acid encoding the desired protease is operably linked to appropriate expression control sequences for each host. Appropriate control sequences useful in any particular expression system are well known to those of skill in the art.

Polynucleotides encoding proteases, recombinant expression vectors, and host cells containing the recombinant expression vectors, can be used to produce the proteases of the disclosure. The methods for making and using these materials to produce recombinant proteins are well are well known to those of skill in the art.

The polynucleotides encoding proteases may be synthesized or prepared by techniques well known in the art. Nucleotide sequences encoding the proteases of the disclosure may be synthesized, and/or cloned, and expressed according to techniques well known to those of ordinary skill in the art. In some embodiments, the polynucleotide sequences will be codon optimized for a particular host cell using standard methodologies. Exemplified polynucleotide sequences codon optimized for expression in E. coli are provided.

Once expressed, the recombinant proteases can be purified according to standard procedures of the art, including ammonium sulfate precipitation, affinity columns, column chromatography, gel electrophoresis and the like. In a typical embodiment, the recombinantly produced protease is expressed as a fusion protein that has a “tag” at one end which facilitates purification of the polypeptide. Suitable tags include epitope tags and affinity tags such as a polyhistidine tag which will bind to metal ions such as nickel or cobalt ions.

For legume source proteins, Protease 1 (SEQ ID NO: 2), Protease 2 (SEQ ID NO: 4), Protease 4 (SEQ ID NO: 8), Protease 5 (SEQ ID NO: 10), Protease 6 (SEQ ID NO: 12), Protease 7 (SEQ ID NO: 14), Protease 8 (SEQ ID NO: 16), Protease 9 (SEQ ID NO: 18), Protease 10 (SEQ ID NO: 20), Protease 11 (SEQ ID NO: 22), and Protease 12 (SEQ ID NO: 24), show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W, F are present.
At position 405, G is present.
At position 437, A, S are present.
At position 438, A, S are present.
At position 439, G is present.
At position 440, D is present.
At position 441, A, E, D, H, N, S are present.
At position 460, S, D, N are present.
At position 572, G is present.
At position 573, G is present.
At position 574, T is present.
At position 575, S is present.
At position 576, A, L are present.

For animal source proteins, Protease 1 (SEQ ID NO: 2), Protease 2 (SEQ ID NO: 4), Protease 4 (SEQ ID NO: 8), Protease 5 (SEQ ID NO: 10), Protease 6 (SEQ ID NO: 12), Protease 7 (SEQ ID NO: 14), Protease 8 (SEQ ID NO: 16), Protease 9 (SEQ ID NO: 18), Protease 11 (SEQ ID NO: 22), Protease 12 (SEQ ID NO: 24), show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W, F are present.
At position 405, G is present.
At position 437, A, S are present.
At position 438, A, S are present.
At position 439, G is present.
At position 440, D is present.
At position 441, A, S, E, D, N are present.
At position 460, S, D are present.
At position 572, G is present.
At position 573, G is present.
At position 574, T is present.
At position 575, S is present.
At position 576, A, L are present.

For non-legume plant source proteins, Protease 1 (SEQ ID NO: 2), Protease 2 (SEQ ID NO: 4), Protease 4 (SEQ ID NO: 8), Protease 5 (SEQ ID NO: 10), Protease 6 (SEQ ID NO: 12), Protease 7 (SEQ ID NO: 14), Protease 8 (SEQ ID NO: 16), Protease 9 (SEQ ID NO: 18), Protease 11 (SEQ ID NO: 22), Protease 12 (SEQ ID NO: 24), show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W, F are present.
At position 405, G is present.
At position 437, A, S are present.
At position 438, A, S are present.
At position 439, G is present.
At position 440, D is present.
At position 441, A, S, E, D, N are present.
At position 460, S, D are present.
At position 572, G is present.
At position 573, G is present.
At position 574, T is present.
At position 575, S is present.
At position 576, A, L are present.
For Each Individual Food Source:

For Mung beans, Protease 1 (SEQ ID NO: 2), Protease 2 (SEQ ID NO: 4), Protease 8 (SEQ ID NO: 16), Protease 9 (SEQ ID NO: 18) show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W are present.
At position 405, G is present.
At position 437, A, S are present.
At position 438, A, S are present.
At position 439, G is present.
At position 440, D is present.
At position 441, S, E, D are present.
At position 460, S, D are present.
At position 572, G is present.
At position 573, G is present.
At position 574, T is present.
At position 575, S is present.
At position 576, A, L are present.

For Green beans, Protease 2 (SEQ ID NO: 4), Protease 6 (SEQ ID NO: 12), Protease 8 (SEQ ID NO: 16), Protease 9 (SEQ ID NO: 18) show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W are present.
At position 405, G is present.
At position 437, A, S are present.
At position 438, A, S are present.
At position 439, G is present.
At position 440, D is present.
At position 441, S, E, D are present.
At position 460, S, D are present.
At position 572, G is present.
At position 573, G is present.
At position 574, T is present.
At position 575, S is present.
At position 576, A, L are present.

For Kidney beans, Protease 2 (SEQ ID NO: 4), Protease 4 (SEQ ID NO: 8), Protease 5 (SEQ ID NO: 10), Protease 6 (SEQ ID NO: 12), Protease 8 (SEQ ID NO: 16), Protease 9 show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W are present.
At position 405, G is present.
At position 437, A, S are present.
At position 438, A, S are present.
At position 439, G is present.
At position 440, D is present.
At position 441, S, E, D are present.
At position 460, S, D are present.
At position 572, G is present.
At position 573, G is present.
At position 574, T is present.
At position 575, S is present.
At position 576, A, L are present.

For Pea, Protease 1 (SEQ ID NO: 2), Protease 2 (SEQ ID NO: 4), Protease 4 (SEQ ID NO: 8), Protease 5 (SEQ ID NO: 10), Protease 6 (SEQ ID NO: 12), Protease 7 (SEQ ID NO: 14), Protease 8 (SEQ ID NO: 16), Protease 9 (SEQ ID NO: 18), Protease 11 (SEQ ID NO: 22), Protease 12 (SEQ ID NO: 24), show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W, F are present.
At position 405, G is present.
At position 437, A, S are present.
At position 438, A, S are present.
At position 439, G is present.
At position 440, D is present.
At position 441, A, S, E, D, N are present.
At position 460, S, D are present.
At position 572, G is present.
At position 573, G is present.
At position 574, T is present.
At position 575, S is present.
At position 576, A, L are present.

For Pinto beans, Protease 2 (SEQ ID NO: 4), Protease 6 (SEQ ID NO: 12), Protease 8 (SEQ ID NO: 16), Protease 9 (SEQ ID NO: 18) show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W are present.
At position 405, G is present.
At position 437, A, S are present.
At position 438, A, S are present.
At position 439, G is present.
At position 440, D is present.
At position 441, S, E, D are present.
At position 460, S, D are present.
At position 572, G is present.
At position 573, G is present.
At position 574, T is present.
At position 575, S is present.
At position 576, A, L are present.

For Black beans, Protease 9 (SEQ ID NO: 18) show activities. Their active site amino acid identities are as follows. The position numbering refers to the corresponding amino acid positions in the alignment shown in FIG. 2.
At position 346, E is present.
At position 380, L is present.
At position 403, S is present.
At position 404, L is present.
At position 405, G is present.
At position 437, S is present.
At position 438, S is present.
At position 439, G is present.
At position 440, D is present.
At position 441, E is present.
At position 460, S is present.
At position 572, G is present.
At position 573, G is present.
At position 574, T is present.
At position 575, S is present.
At position 576, L is present.

For Lentil, Protease 2 (SEQ ID NO: 4), Protease 6 (SEQ ID NO: 12), Protease 8 (SEQ ID NO: 16), Protease 9 (SEQ ID NO: 18) show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W are present.
At position 405, G is present.
At position 437, A, S are present.
At position 438, A, S are present.
At position 439, G is present.
At position 440, D is present.
At position 441, S, E, D are present.
At position 460, S, D are present.
At position 572, G is present.
At position 573, G is present.
At position 574, T is present.
At position 575, S is present.
At position 576, A, L are present.

For Chickpea, Protease 1 (SEQ ID NO: 2), Protease 2 (SEQ ID NO: 4), Protease 4 (SEQ ID NO: 8), Protease 5 (SEQ ID NO: 10), Protease 6 (SEQ ID NO: 12), Protease 8 (SEQ ID NO: 16), Protease 9 (SEQ ID NO: 18), Protease 10 (SEQ ID NO: 20), Protease 11 (SEQ ID NO: 22), Protease 12 (SEQ ID NO: 24), show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W are present.
At position 405, G is present.
At position 437, A, S are present.
At position 438, A, S are present.
At position 439, G is present.
At position 440, D is present.
At position 441, H, S, E, D, N are present.
At position 460, S, D, N are present.
At position 572, G is present.
At position 573, G is present.
At position 574, T is present.
At position 575, S is present.
At position 576, A, L are present.

For Lupine Beans, Protease 1 (SEQ ID NO: 2), Protease 2 (SEQ ID NO: 4), Protease 8 (SEQ ID NO: 16), Protease 11 (SEQ ID NO: 22), Protease 12 (SEQ ID NO: 24), show activities. Their active site amino acid identities are as follows. The position numbering refers to the corresponding amino acid positions in the alignment shown in FIG. 2.
At position 346, E is present.
At position 380, F is present.
At position 403, S is present.
At position 404, W is present.
At position 405, G is present.
At position 437, A is present.
At position 438, A, S are present.
At position 439, G is present.
At position 440, D is present.
At position 441, S, D, N are present.
At position 460, D is present.
At position 572, G is present.
At position 573, G is present.
At position 574, T is present.
At position 575, S is present.
At position 576, A is present.

For Field Peas, Protease 9 (SEQ ID NO: 18) show activities. Their active site amino acid identities are as follows. The position numbering refers to the corresponding amino acid positions in the alignment shown in FIG. 2.
At position 346, E is present.
At position 380, L is present.
At position 403, S is present.
At position 404, L is present.
At position 405, G is present.
At position 437, S is present.
At position 438, S is present.
At position 439, G is present.
At position 440, D is present.
At position 441, E is present.
At position 460, S is present.
At position 572, G is present.
At position 573, G is present.
At position 574, T is present.
At position 575, S is present.
At position 576, L is present.

For Cowpea, Protease 9 (SEQ ID NO: 18) show activities. Their active site amino acid identities are as follows. The position numbering refers to the corresponding amino acid positions in the alignment shown in FIG. 2.
At position 346, E is present.
At position 380, L is present.
At position 403, S is present.
At position 404, L is present.
At position 405, G is present.
At position 437, S is present.
At position 438, S is present.
At position 439, G is present.
At position 440, D is present.
At position 441, E is present.
At position 460, S is present.
At position 572, G is present.
At position 573, G is present.
At position 574, T is present.
At position 575, S is present.
At position 576, L is present.

For Baby Lima, Protease 1 (SEQ ID NO: 2), Protease 2 (SEQ ID NO: 4), Protease 4 (SEQ ID NO: 8), Protease 5 (SEQ ID NO: 10), Protease 6 (SEQ ID NO: 12), Protease 7 (SEQ ID NO: 14), Protease 8 (SEQ ID NO: 16), Protease 9 (SEQ ID NO: 18) show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W, F are present.
At position 405, G is present.
At position 437, A, S are present.
At position 438, A, S are present.
At position 439, G is present.
At position 440, D is present.
At position 441, A, S, E, D are present.
At position 460, S, D are present.
At position 572, G is present.
At position 573, G is present.
At position 574, T is present.
At position 575, S is present.
At position 576, A, L are present.

For Crowder pea, Protease 9 (SEQ ID NO: 18), Protease 11 (SEQ ID NO: 22), Protease 12 (SEQ ID NO: 24), show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W are present.
At position 405, G is present.
At position 437, A, S are present.
At position 438, A, S are present.
At position 439, G is present.
At position 440, D is present.
At position 441, E, N are present.
At position 460, S, D are present.
At position 572, G is present.
At position 573, G is present.
At position 574, T is present.
At position 575, S is present.
At position 576, A, L are present.

For Pink beans, Protease 1 (SEQ ID NO: 2), Protease 2 (SEQ ID NO: 4), Protease 6 (SEQ ID NO: 12), Protease 9 (SEQ ID NO: 18) show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W are present.
At position 405, G is present.
At position 437, A, S are present.
At position 438, A, S are present.
At position 439, G is present.
At position 440, D is present.
At position 441, S, E, D are present.
At position 460, S, D are present.
At position 572, G is present.
At position 573, G is present.
At position 574, T is present.
At position 575, S is present.
At position 576, A, L are present.

For Adzuki beans, Protease 9 (SEQ ID NO: 18) show activities. Their active site amino acid identities are as follows. The position numbering refers to the corresponding amino acid positions in the alignment shown in FIG. 2.
At position 346, E is present.
At position 380, L is present.
At position 403, S is present.
At position 404, L is present.
At position 405, G is present.
At position 437, S is present.
At position 438, S is present.
At position 439, G is present.
At position 440, D is present.
At position 441, E is present.
At position 460, S is present.
At position 572, G is present.
At position 573, G is present.
At position 574, T is present.
At position 575, S is present.
At position 576, L is present.

For Lady cream peas, Protease 9 (SEQ ID NO: 18) show activities. Their active site amino acid identities are as follows. The position numbering refers to the corresponding amino acid positions in the alignment shown in FIG. 2.
At position 346, E is present.
At position 380, L is present.
At position 403, S is present.
At position 404, L is present.
At position 405, G is present.
At position 437, S is present.
At position 438, S is present.
At position 439, G is present.
At position 440, D is present.
At position 441, E is present.
At position 460, S is present.
At position 572, G is present.
At position 573, G is present.
At position 574, T is present.
At position 575, S is present.
At position 576, L is present.

For Cannelinni beans, Protease 2 (SEQ ID NO: 4), Protease 8 (SEQ ID NO: 16), Protease 9 (SEQ ID NO: 18) show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W are present.
At position 405, G is present.
At position 437, A, S are present.
At position 438, A, S are present.
At position 439, G is present.
At position 440, D is present.
At position 441, S, E, D are present.
At position 460, S, D are present.
At position 572, G is present.
At position 573, G is present.
At position 574, T is present.
At position 575, S is present.
At position 576, A, L are present.

For Pigeon Peas, Protease 1 (SEQ ID NO: 2), Protease 2 (SEQ ID NO: 4), Protease 4 (SEQ ID NO: 8), Protease 5 (SEQ ID NO: 10), Protease 6 (SEQ ID NO: 12), Protease 7 (SEQ ID NO: 14), Protease 8 (SEQ ID NO: 16), Protease 9 (SEQ ID NO: 18) show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W, F are present.
At position 405, G is present.
At position 437, A, S are present.
At position 438, A, S are present.
At position 439, G is present.
At position 440, D is present.
At position 441, A, S, E, D are present.
At position 460, S, D are present.
At position 572, G is present.
At position 573, G is present.
At position 574, T is present.
At position 575, S is present.
At position 576, A, L are present.

For Yellow split peas, Protease 1 (SEQ ID NO: 2), Protease 2 (SEQ ID NO: 4), Protease 4 (SEQ ID NO: 8), Protease 5 (SEQ ID NO: 10), Protease 6 (SEQ ID NO: 12), Protease 7 (SEQ ID NO: 14), Protease 8 (SEQ ID NO: 16), Protease 9 (SEQ ID NO: 18) show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W, F are present.
At position 405, G is present.
At position 437, A, S are present.
At position 438, A, S are present.
At position 439, G is present.
At position 440, D is present.
At position 441, A, S, E, D are present.
At position 460, S, D are present.
At position 572, G is present.
At position 573, G is present.
At position 574, T is present.
At position 575, S is present.
At position 576, A, L are present.

For Navy pea, Protease 9 (SEQ ID NO: 18) show activities. Their active site amino acid identities are as follows. The position numbering refers to the corresponding amino acid positions in the alignment shown in FIG. 2.
At position 346, E is present.
At position 380, L is present.
At position 403, S is present.
At position 404, L is present.
At position 405, G is present.
At position 437, S is present.
At position 438, S is present.
At position 439, G is present.
At position 440, D is present.
At position 441, E is present.
At position 460, S is present.
At position 572, G is present.
At position 573, G is present.
At position 574, T is present.
At position 575, S is present.
At position 576, L is present.

For Black-eyed peas, Protease 9 (SEQ ID NO: 18) show activities. Their active site amino acid identities are as follows. The position numbering refers to the corresponding amino acid positions in the alignment shown in FIG. 2.
At position 346, E is present.
At position 380, L is present.
At position 403, S is present.
At position 404, L is present.
At position 405, G is present.
At position 437, S is present.
At position 438, S is present.
At position 439, G is present.
At position 440, D is present.
At position 441, E is present.
At position 460, S is present.
At position 572, G is present.
At position 573, G is present.
At position 574, T is present.
At position 575, S is present.
At position 576, L is present.

For Masdoor Dal (Indian Red lentils), Protease 2 (SEQ ID NO: 4), Protease 9 (SEQ ID NO: 18) show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W are present.
At position 405, G is present.
At position 437, A, S are present.
At position 438, A, S are present.
At position 439, G is present.
At position 440, D is present.
At position 441, E, D are present.
At position 460, S, D are present.
At position 572, G is present.
At position 573, G is present.
At position 574, T is present.
At position 575, S is present.
At position 576, A, L are present.

For Great Northern Beans, Protease 2 (SEQ ID NO: 4), Protease 8 (SEQ ID NO: 16), Protease 9 (SEQ ID NO: 18) show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W are present.
At position 405, G is present.
At position 437, A, S are present.
At position 438, A, S are present.
At position 439, G is present.
At position 440, D is present.
At position 441, S, E, D are present.
At position 460, S, D are present.
At position 572, G is present.
At position 573, G is present.
At position 574, T is present.
At position 575, S is present.
At position 576, A, L are present.

For Cranberry beans, Protease 9 (SEQ ID NO: 18) show activities. Their active site amino acid identities are as follows. The position numbering refers to the corresponding amino acid positions in the alignment shown in FIG. 2.
At position 346, E is present.
At position 380, L is present.
At position 403, S is present.
At position 404, L is present.
At position 405, G is present.
At position 437, S is present.
At position 438, S is present.
At position 439, G is present.
At position 440, D is present.
At position 441, E is present.
At position 460, S is present.
At position 572, G is present.
At position 573, G is present.
At position 574, T is present.
At position 575, S is present.
At position 576, L is present.

For White beans, Protease 1 (SEQ ID NO: 2), Protease 2 (SEQ ID NO: 4), Protease 4 (SEQ ID NO: 8), Protease 5 (SEQ ID NO: 10), Protease 6 (SEQ ID NO: 12), Protease 7 (SEQ ID NO: 14), Protease 8 (SEQ ID NO: 16), Protease 9 (SEQ ID NO: 18) show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W, F are present.
At position 405, G is present.
At position 437, A, S are present.
At position 438, A, S are present.
At position 439, G is present.
At position 440, D is present.
At position 441, A, S, E, D are present.
At position 460, S, D are present.
At position 572, G is present.
At position 573, G is present.
At position 574, T is present.
At position 575, S is present.
At position 576, A, L are present.

For Fava beans, Protease 1 (SEQ ID NO: 2), Protease 2 (SEQ ID NO: 4), Protease 8 (SEQ ID NO: 16), Protease 9 (SEQ ID NO: 18) show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W are present.
At position 405, G is present.
At position 437, A, S are present.
At position 438, A, S are present.
At position 439, G is present.
At position 440, D is present.
At position 441, S, E, D are present.
At position 460, S, D are present.
At position 572, G is present.
At position 573, G is present.
At position 574, T is present.
At position 575, S is present.
At position 576, A, L are present.

For Salmon, Protease 1 (SEQ ID NO: 2), Protease 2 (SEQ ID NO: 4), Protease 8 (SEQ ID NO: 16), Protease 9 (SEQ ID NO: 18) show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W are present.
At position 405, G is present.
At position 437, A, S are present.
At position 438, A, S are present.
At position 439, G is present.
At position 440, D is present.
At position 441, S, E, D are present.
At position 460, S, D are present.
At position 572, G is present.
At position 573, G is present.
At position 574, T is present.
At position 575, S is present.
At position 576, A, L are present.

For Pork, Protease 1 (SEQ ID NO: 2), Protease 2 (SEQ ID NO: 4), Protease 4 (SEQ ID NO: 8), Protease 5 (SEQ ID NO: 10), Protease 6 (SEQ ID NO: 12), Protease 7 (SEQ ID NO: 14), Protease 8 (SEQ ID NO: 16), Protease 9 (SEQ ID NO: 18) show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W, F are present.
At position 405, G is present.
At position 437, A, S are present.
At position 438, A, S are present.
At position 439, G is present.
At position 440, D is present.
At position 441, A, S, E, D are present.
At position 460, S, D are present.
At position 572, G is present.
At position 573, G is present.
At position 574, T is present.
At position 575, S is present.
At position 576, A, L are present.

For Chicken, Protease 1 (SEQ ID NO: 2), Protease 2 (SEQ ID NO: 4), Protease 4 (SEQ ID NO: 8), Protease 8 (SEQ ID NO: 16), Protease 9 (SEQ ID NO: 18) show activities.

Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W are present.
At position 405, G is present.
At position 437, A, S are present.
At position 438, A, S are present.
At position 439, G is present.
At position 440, D is present.
At position 441, S, E, D are present.
At position 460, S, D are present.
At position 572, G is present.
At position 573, G is present.
At position 574, T is present.
At position 575, S is present.
At position 576, A, L are present.

For Turkey, Protease 1 (SEQ ID NO: 2), Protease 2 (SEQ ID NO: 4), Protease 4 (SEQ ID NO: 8), Protease 5 (SEQ ID NO: 10), Protease 6 (SEQ ID NO: 12), Protease 7 (SEQ ID NO: 14), Protease 8 (SEQ ID NO: 16), Protease 9 (SEQ ID NO: 18) show activities.

Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W, F are present.
At position 405, G is present.
At position 437, A, S are present.
At position 438, A, S are present.
At position 439, G is present.
At position 440, D is present.
At position 441, A, S, E, D are present.
At position 460, S, D are present.
At position 572, G is present.
At position 573, G is present.
At position 574, T is present.
At position 575, S is present.
At position 576, A, L are present.

For Beef, Protease 2 (SEQ ID NO: 4), Protease 8 (SEQ ID NO: 16), Protease 9 (SEQ ID NO: 18) show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W are present.
At position 405, G is present.
At position 437, A, S are present.
At position 438, A, S are present.
At position 439, G is present.
At position 440, D is present.
At position 441, S, E, D are present.
At position 460, S, D are present.
At position 572, G is present.
At position 573, G is present.
At position 574, T is present.
At position 575, S is present.
At position 576, A, L are present.

For Flounder, Protease 2 (SEQ ID NO: 4), Protease 8 (SEQ ID NO: 16), Protease 9 (SEQ ID NO: 18), Protease 11 show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W are present.
At position 405, G is present.
At position 437, A, S are present.
At position 438, A, S are present.
At position 439, G is present.
At position 440, D is present.
At position 441, S, E, D, N are present.
At position 460, S, D are present.
At position 572, G is present.
At position 573, G is present.
At position 574, T is present.
At position 575, S is present.
At position 576, A, L are present.

For Yogurt, Protease 9 (SEQ ID NO: 18) show activities. Their active site amino acid identities are as follows. The position numbering refers to the corresponding amino acid positions in the alignment shown in FIG. 2.
At position 346, E is present.
At position 380, L is present.
At position 403, S is present.
At position 404, L is present.
At position 405, G is present.
At position 437, S is present.
At position 438, S is present.
At position 439, G is present.
At position 440, D is present.
At position 441, E is present.
At position 460, S is present.
At position 572, G is present.
At position 573, G is present.
At position 574, T is present.
At position 575, S is present.
At position 576, L is present.

For Asparagus, Protease 1 (SEQ ID NO: 2), Protease 2 (SEQ ID NO: 4), Protease 4 (SEQ ID NO: 8), Protease 5 (SEQ ID NO: 10), Protease 6 (SEQ ID NO: 12), Protease 7 (SEQ ID NO: 14), Protease 8 (SEQ ID NO: 16), Protease 9 (SEQ ID NO: 18), Protease 11 (SEQ ID NO: 22), Protease 12 (SEQ ID NO: 24), show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W, F are present.
At position 405, G is present.
At position 437, A, S are present.
At position 438, A, S are present.
At position 439, G is present.
At position 440, D is present.
At position 441, A, S, E, D, N are present.
At position 460, S, D are present.
At position 572, G is present.
At position 573, G is present.
At position 574, T is present.
At position 575, S is present.
At position 576, A, L are present.

For Whey, Protease 2 (SEQ ID NO: 4), Protease 9 (SEQ ID NO: 18) show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W are present.
At position 405, G is present.
At position 437, A, S are present.
At position 438, A, S are present.
At position 439, G is present.
At position 440, D is present.
At position 441, E, D are present.
At position 460, S, D are present.
At position 572, G is present.
At position 573, G is present.
At position 574, T is present.
At position 575, S is present.
At position 576, A, L are present.

For Casein, Protease 4 (SEQ ID NO: 8), Protease 5 (SEQ ID NO: 10), Protease 6 (SEQ ID NO: 12), Protease 7 (SEQ ID NO: 14), Protease 8 (SEQ ID NO: 16), Protease 9 (SEQ ID NO: 18), Protease 11 (SEQ ID NO: 22) show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W, F are present.
At position 405, G is present.
At position 437, A, S are present.
At position 438, A, S are present.
At position 439, G is present.
At position 440, D is present.
At position 441, A, S, E, N are present.
At position 460, S, D are present.
At position 572, G is present.
At position 573, G is present.
At position 574, T is present.
At position 575, S is present.
At position 576, A, L are present.

For Pea Protein powder, Protease 1 (SEQ ID NO: 2), Protease 2 (SEQ ID NO: 4), Protease 4 (SEQ ID NO: 8), Protease 5 (SEQ ID NO: 10), Protease 6 (SEQ ID NO: 12), Protease 7 (SEQ ID NO: 14), Protease 8 (SEQ ID NO: 16), Protease 9 (SEQ ID NO: 18) show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W, F are present.
At position 405, G is present.
At position 437, A, S are present.
At position 438, A, S are present.
At position 439, G is present.
At position 440, D is present.
At position 441, A, S, E, D are present.
At position 460, S, D are present.
At position 572, G is present.
At position 573, G is present.
At position 574, T is present.
At position 575, S is present.
At position 576, A, L are present.

For Vicillin, Protease 9 (SEQ ID NO: 18) show activities. Their active site amino acid identities are as follows. The position numbering refers to the corresponding amino acid positions in the alignment shown in FIG. 2.
At position 346, E is present.
At position 380, L is present.
At position 403, S is present.
At position 404, L is present.
At position 405, G is present.
At position 437, S is present.
At position 438, S is present.
At position 439, G is present.
At position 440, D is present.
At position 441, E is present.
At position 460, S is present.
At position 572, G is present.
At position 573, G is present.
At position 574, T is present.
At position 575, S is present.
At position 576, L is present.

For Soy, Protease 1 (SEQ ID NO: 2), Protease 2 (SEQ ID NO: 4), Protease 4 (SEQ ID NO: 8), Protease 5 (SEQ ID NO: 10), Protease 6 (SEQ ID NO: 12), Protease 7 (SEQ ID NO: 14), Protease 8 (SEQ ID NO: 16), Protease 9 (SEQ ID NO: 18), Protease 10 (SEQ ID NO: 20), Protease 11 (SEQ ID NO: 22), Protease 12 (SEQ ID NO: 24), show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W, F are present.
At position 405, G is present.
At position 437, A, S are present.
At position 438, A, S are present.
At position 439, G is present.
At position 440, D is present.
At position 441, A, E, D, H, N, S are present.
At position 460, S, D, N are present.
At position 572, G is present.
At position 573, G is present.
At position 574, T is present.
At position 575, S is present.
At position 576, A, L are present.

For Hemp protein powder, Protease 2 (SEQ ID NO: 4), Protease 8 (SEQ ID NO: 16), Protease 9 (SEQ ID NO: 18) show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W are present.
At position 405, G is present.
At position 437, A, S are present.
At position 438, A, S are present.
At position 439, G is present.
At position 440, D is present.
At position 441, S, E, D are present.
At position 460, S, D are present.
At position 572, G is present.
At position 573, G is present.
At position 574, T is present.
At position 575, S is present.
At position 576, A, L are present.

For Broccoli, Protease 1 (SEQ ID NO: 2), Protease 2 (SEQ ID NO: 4), Protease 4 (SEQ ID NO: 8), Protease 5 (SEQ ID NO: 10), Protease 6 (SEQ ID NO: 12), Protease 7 (SEQ ID NO: 14), Protease 8 (SEQ ID NO: 16), Protease 9 (SEQ ID NO: 18), Protease 11 (SEQ ID NO: 22), Protease 12 (SEQ ID NO: 24), show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W, F are present.
At position 405, G is present.
At position 437, A, S are present.
At position 438, A, S are present.
At position 439, G is present.
At position 440, D is present.
At position 441, A, S, E, D, N are present.
At position 460, S, D are present.
At position 572, G is present.
At position 573, G is present.
At position 574, T is present.
At position 575, S is present.
At position 576, A, L are present.

For Quinoa, Protease 2 (SEQ ID NO: 4), Protease 8 (SEQ ID NO: 16) show activities. Their active site amino acid identities are as follows. The position numbering refers to the corresponding amino acid positions in the alignment shown in FIG. 2.
At position 346, E is present.
At position 380, F is present.
At position 403, S is present.
At position 404, W is present.
At position 405, G is present.
At position 437, A is present.
At position 438, A is present.
At position 439, G is present.
At position 440, D is present.
At position 441, S, D are present.
At position 460, D is present.
At position 572, G is present.
At position 573, G is present.
At position 574, T is present.
At position 575, S is present.
At position 576, A is present.

For Buckwheat, Protease 1 (SEQ ID NO: 2), Protease 2 (SEQ ID NO: 4), Protease 4 (SEQ ID NO: 8), Protease 5 (SEQ ID NO: 10), Protease 6 (SEQ ID NO: 12), Protease 7 (SEQ ID NO: 14), Protease 8 (SEQ ID NO: 16), Protease 9 (SEQ ID NO: 18) show activities.

Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W, F are present.
At position 405, G is present.
At position 437, A, S are present.
At position 438, A, S are present.
At position 439, G is present.
At position 440, D is present.
At position 441, A, S, E, D are present.
At position 460, S, D are present.
At position 572, G is present.
At position 573, G is present.
At position 574, T is present.
At position 575, S is present.
At position 576, A, L are present.

For Chia seeds, Protease 2 (SEQ ID NO: 4), Protease 5 (SEQ ID NO: 10), Protease 6 (SEQ ID NO: 12), Protease 8 (SEQ ID NO: 16), Protease 9 (SEQ ID NO: 18), Protease 11 (SEQ ID NO: 22) show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W are present.
At position 405, G is present.
At position 437, A, S are present.
At position 438, A, S are present.
At position 439, G is present.
At position 440, D is present.
At position 441, S, E, D, N are present.
At position 460, S, D are present.
At position 572, G is present.
At position 573, G is present.
At position 574, T is present.
At position 575, S is present.
At position 576, A, L are present.

For Kamut, Protease 1 (SEQ ID NO: 2), Protease 2 (SEQ ID NO: 4), Protease 4 (SEQ ID NO: 8), Protease 5 (SEQ ID NO: 10), Protease 6 (SEQ ID NO: 12), Protease 7 (SEQ ID NO: 14), Protease 8 (SEQ ID NO: 16), Protease 9 (SEQ ID NO: 18), Protease 11 (SEQ ID NO: 22), Protease 12 (SEQ ID NO: 24), show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W, F are present.
At position 405, G is present.
At position 437, A, S are present.
At position 438, A, S are present.
At position 439, G is present.
At position 440, D is present.
At position 441, A, S, E, D, N are present.
At position 460, S, D are present.
At position 572, G is present.
At position 573, G is present.
At position 574, T is present.
At position 575, S is present.
At position 576, A, L are present.

For Rye berries, Protease 1 (SEQ ID NO: 2), Protease 2 (SEQ ID NO: 4), Protease 9 (SEQ ID NO: 18), Protease 11 (SEQ ID NO: 22) show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W are present.
At position 405, G is present.
At position 437, A, S are present.
At position 438, A, S are present.
At position 439, G is present.
At position 440, D is present.
At position 441, E, D, N are present.
At position 460, S, D are present.
At position 572, G is present.
At position 573, G is present.
At position 574, T is present.
At position 575, S is present.
At position 576, A, L are present.

For Amaranth, Protease 1 (SEQ ID NO: 2), Protease 2 (SEQ ID NO: 4), Protease 8 (SEQ ID NO: 16), Protease 9 (SEQ ID NO: 18) show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W are present.
At position 405, G is present.
At position 437, A, S are present.
At position 438, A, S are present.
At position 439, G is present.
At position 440, D is present.
At position 441, S, E, D are present.
At position 460, S, D are present.
At position 572, G is present.
At position 573, G is present.
At position 574, T is present.
At position 575, S is present.
At position 576, A, L are present.

For Barley, Protease 1 (SEQ ID NO: 2), Protease 2 (SEQ ID NO: 4), Protease 4 (SEQ ID NO: 8), Protease 5 (SEQ ID NO: 10), Protease 6 (SEQ ID NO: 12), Protease 7 (SEQ ID NO: 14), Protease 8 (SEQ ID NO: 16), Protease 9 (SEQ ID NO: 18) show activities.

Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W, F are present.
At position 405, G is present.
At position 437, A, S are present.
At position 438, A, S are present.
At position 439, G is present.
At position 440, D is present.
At position 441, A, S, E, D are present.
At position 460, S, D are present.
At position 572, G is present.
At position 573, G is present.
At position 574, T is present.
At position 575, S is present.
At position 576, A, L are present.

For Chicken Egg, Protease 2 (SEQ ID NO: 4), Protease 9 (SEQ ID NO: 18) show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W are present.
At position 405, G is present.
At position 437, A, S are present.
At position 438, A, S are present.
At position 439, G is present.
At position 440, D is present.
At position 441, E, D are present.
At position 460, S, D are present.
At position 572, G is present.
At position 573, G is present.
At position 574, T is present.
At position 575, S is present.
At position 576, A, L are present.

For Spirulina, Protease 1 (SEQ ID NO: 2), Protease 2 (SEQ ID NO: 4), Protease 4 (SEQ ID NO: 8), Protease 5 (SEQ ID NO: 10), Protease 6 (SEQ ID NO: 12), Protease 7 (SEQ ID NO: 14), Protease 8 (SEQ ID NO: 16), Protease 9 (SEQ ID NO: 18) show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W, F are present.
At position 405, G is present.
At position 437, A, S are present.
At position 438, A, S are present.
At position 439, G is present.
At position 440, D is present.
At position 441, A, S, E, D are present.
At position 460, S, D are present.
At position 572, G is present.
At position 573, G is present.
At position 574, T is present.
At position 575, S is present.
At position 576, A, L are present.

For Chlorella, Protease 9 (SEQ ID NO: 18) show activities. Their active site amino acid identities are as follows. The position numbering refers to the corresponding amino acid positions in the alignment shown in FIG. 2.
At position 346, E is present.
At position 380, L is present.
At position 403, S is present.
At position 404, L is present.
At position 405, G is present.
At position 437, S is present.
At position 438, S is present.
At position 439, G is present.
At position 440, D is present.
At position 441, E is present.
At position 460, S is present.
At position 572, G is present.
At position 573, G is present.
At position 574, T is present.
At position 575, S is present.
At position 576, L is present.

For Peanut, Protease 2 (SEQ ID NO: 4), Protease 9 (SEQ ID NO: 18) show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W are present.
At position 405, G is present.
At position 437, A, S are present.
At position 438, A, S are present.
At position 439, G is present.
At position 440, D is present.
At position 441, E, D are present.
At position 460, S, D are present.
At position 572, G is present.
At position 573, G is present.
At position 574, T is present.
At position 575, S is present.
At position 576, A, L are present.

For Sunflower seeds, Protease 2 (SEQ ID NO: 4) show activities. Their active site amino acid identities are as follows. The position numbering refers to the corresponding amino acid positions in the alignment shown in FIG. 2.
At position 346, E is present.
At position 380, F is present.
At position 403, S is present.
At position 404, W is present.
At position 405, G is present.
At position 437, A is present.
At position 438, A is present.
At position 439, G is present.
At position 440, D is present.
At position 441, D is present.
At position 460, D is present.
At position 572, G is present.
At position 573, G is present.
At position 574, T is present.
At position 575, S is present.
At position 576, A is present.

For Almonds, Protease 2 (SEQ ID NO: 4), Protease 8 (SEQ ID NO: 16), Protease 9 (SEQ ID NO: 18) show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W are present.
At position 405, G is present.
At position 437, A, S are present.
At position 438, A, S are present.
At position 439, G is present.
At position 440, D is present.
At position 441, S, E, D are present.
At position 460, S, D are present.
At position 572, G is present.
At position 573, G is present.
At position 574, T is present.
At position 575, S is present.
At position 576, A, L are present.

For Cashews, Protease 2 (SEQ ID NO: 4), Protease 9 (SEQ ID NO: 18) show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W are present.
At position 405, G is present.
At position 437, A, S are present.
At position 438, A, S are present.
At position 439, G is present.
At position 440, D is present.
At position 441, E, D are present.
At position 460, S, D are present.
At position 572, G is present.
At position 573, G is present.
At position 574, T is present.
At position 575, S is present.
At position 576, A, L are present.

For Pistachios, Protease 9 (SEQ ID NO: 18) show activities. Their active site amino acid identities are as follows. The position numbering refers to the corresponding amino acid positions in the alignment shown in FIG. 2.
At position 346, E is present.
At position 380, L is present.
At position 403, S is present.
At position 404, L is present.
At position 405, G is present.
At position 437, S is present.
At position 438, S is present.
At position 439, G is present.
At position 440, D is present.
At position 441, E is present.
At position 460, S is present.
At position 572, G is present.
At position 573, G is present.
At position 574, T is present.
At position 575, S is present.
At position 576, L is present.

For Royal Canin, Protease 8 (SEQ ID NO: 16), Protease 9 (SEQ ID NO: 18) show activities. Their active site amino acid identities are as follows. The position numbering refers to the corresponding amino acid positions in the alignment shown in FIG. 2.
At position 346, E is present.
At position 380, L, F are present.
At position 403, S is present.
At position 404, L, W are present.
At position 405, G is present.
At position 437, A, S are present.
At position 438, A, S are present.
At position 439, G is present.
At position 440, D is present.
At position 441, S, E are present.
At position 460, S, D are present.
At position 572, G is present.
At position 573, G is present.
At position 574, T is present.
At position 575, S is present.
At position 576, A, L are present.

The following shows active site amino acids that are unique to particular proteases:

Active site amino acids that are unique to proteases that are active on Mung beans:
Amino acid “L” at position 404 in the alignment.
Amino acid “E” at position 441 in the alignment.
   Active site amino acids that are unique to proteases that are active on Green beans:
Amino acid “H” at position 441 in the alignment.
Amino acid “L” at position 404 in the alignment.
Amino acid “N” at position 460 in the alignment.
   Active site amino acids that are unique to proteases that are active on Kidney beans:
Amino acid “H” at position 441 in the alignment.
Amino acid “L” at position 404 in the alignment.
Amino acid “N” at position 460 in the alignment.
   Active site amino acids that are unique to proteases that are active on Pea:
Amino acid “H” at position 441 in the alignment.
Amino acid “L” at position 404 in the alignment.
Amino acid “C” at position 460 in the alignment.
Amino acid “A” at position 438 in the alignment.
   Active site amino acids that are unique to proteases that are active on Pinto beans:
Amino acid “H” at position 441 in the alignment.
Amino acid “L” at position 404 in the alignment.
Amino acid “N” at position 460 in the alignment.
   Active site amino acids that are unique to proteases that are active on Black beans:
Amino acid “L” at position 404 in the alignment.
Amino acid “E” at position 441 in the alignment.
   Active site amino acids that are unique to proteases that are active on Lentil:
Amino acid “H” at position 441 in the alignment.
Amino acid “L” at position 404 in the alignment.
Amino acid “N” at position 460 in the alignment.
   Active site amino acids that are unique to proteases that are active on Chickpea:
Amino acid “H” at position 441 in the alignment.
Amino acid “L” at position 404 in the alignment.
Amino acid “D” at position 460 in the alignment.
Amino acid “A” at position 438 in the alignment.
   Active site amino acids that are unique to proteases that are active on Lupine beans:
Amino acid “A” at position 438 in the alignment.
Active site amino acids that are unique to proteases that are active on Field peas
Amino acid “L” at position 404 in the alignment.
Amino acid “E” at position 441 in the alignment.
   Active site amino acids that are unique to proteases that are active on Cowpea:
Amino acid “L” at position 404 in the alignment.
Amino acid “E” at position 441 in the alignment.
   Active site amino acids that are unique to proteases that are active on Baby Lima:
Amino acid “H” at position 441 in the alignment.
Amino acid “L” at position 404 in the alignment.
Amino acid “C” at position 460 in the alignment.
   Active site amino acids that are unique to proteases that are active on Crowder pea:
Amino acid “E” at position 441 in the alignment.
Amino acid “L” at position 404 in the alignment.
   Active site amino acids that are unique to proteases that are active on Pink beans:
Amino acid “H” at position 441 in the alignment.
Amino acid “L” at position 404 in the alignment.
Amino acid “N” at position 460 in the alignment.
   Active site amino acids that are unique to proteases that are active on Adzuki beans:
Amino acid “L” at position 404 in the alignment.
Amino acid “E” at position 441 in the alignment.
   Active site amino acids that are unique to proteases that are active on Lady cream peas:
Amino acid “L” at position 404 in the alignment.
Amino acid “E” at position 441 in the alignment.
   Active site amino acids that are unique to proteases that are active on Canellini beans:
Amino acid “L” at position 404 in the alignment.
Amino acid “E” at position 441 in the alignment.
   Active site amino acids that are unique to proteases that are active on Pigeon peas:
Amino acid “H” at position 441 in the alignment.
Amino acid “L” at position 404 in the alignment.
Amino acid “C” at position 460 in the alignment.
   Active site amino acids that are unique to proteases that are active on Yellow split peas:
Amino acid “H” at position 441 in the alignment.
Amino acid “L” at position 404 in the alignment.
Amino acid “C” at position 460 in the alignment.
   Active site amino acids that are unique to proteases that are active on Navy pea:
Amino acid “L” at position 404 in the alignment.
Amino acid “E” at position 441 in the alignment.
   Active site amino acids that are unique to proteases that are active on Black eyed peas:
Amino acid “L” at position 404 in the alignment.
Amino acid “E” at position 441 in the alignment.
   Active site amino acids that are unique to proteases that are active on Masdoor Dal:
Amino acid “L” at position 404 in the alignment.
Amino acid “E” at position 441 in the alignment.
   Active site amino acids that are unique to proteases that are active on Great Northern Beans:
Amino acid “L” at position 404 in the alignment.
Amino acid “E” at position 441 in the alignment.
   Active site amino acids that are unique to proteases that are active on Cranberry beans:
Amino acid “L” at position 404 in the alignment.
Amino acid “E” at position 441 in the alignment.
   Active site amino acids that are unique to proteases that are active on White beans:
Amino acid “H” at position 441 in the alignment.
Amino acid “L” at position 404 in the alignment.
Amino acid “C” at position 460 in the alignment.
   Active site amino acids that are unique to proteases that are active on Fava beans:
Amino acid “L” at position 404 in the alignment.
Amino acid “E” at position 441 in the alignment.
   Active site amino acids that are unique to proteases that are active on Salmon:
Amino acid “L” at position 404 in the alignment.
Amino acid “E” at position 441 in the alignment.
   Active site amino acids that are unique to proteases that are active on Pork:
Amino acid “H” at position 441 in the alignment.
Amino acid “L” at position 404 in the alignment.
Amino acid “C” at position 460 in the alignment.
   Active site amino acids that are unique to proteases that are active on Chicken:
Amino acid “E” at position 441 in the alignment.
Amino acid “L” at position 404 in the alignment.
   Active site amino acids that are unique to proteases that are active on Turkey
Amino acid “H” at position 441 in the alignment.
Amino acid “L” at position 404 in the alignment.
Amino acid “C” at position 460 in the alignment.
   Active site amino acids that are unique to proteases that are active on Beef:
Amino acid “L” at position 404 in the alignment.
Amino acid “E” at position 441 in the alignment.
   Active site amino acids that are unique to proteases that are active on Flounder:
Amino acid “E” at position 441 in the alignment.
Amino acid “L” at position 404 in the alignment.
   Active site amino acids that are unique to proteases that are active on Yogurt:
Amino acid “L” at position 404 in the alignment.
Amino acid “E” at position 441 in the alignment.
   Active site amino acids that are unique to proteases that are active on Asparagus:
Amino acid “H” at position 441 in the alignment.
Amino acid “L” at position 404 in the alignment.
Amino acid “C” at position 460 in the alignment.
Amino acid “A” at position 438 in the alignment.
   Active site amino acids that are unique to proteases that are active on Whey:
Amino acid “L” at position 404 in the alignment.
Amino acid “E” at position 441 in the alignment.
   Active site amino acids that are unique to proteases that are active on Casein:
Amino acid “H” at position 441 in the alignment.
Amino acid “L” at position 404 in the alignment.
Amino acid “C” at position 460 in the alignment.
   Active site amino acids that are unique to proteases that are active on Pea protein powder:
Amino acid “H” at position 441 in the alignment.
Amino acid “L” at position 404 in the alignment.
Amino acid “C” at position 460 in the alignment.
   Active site amino acids that are unique to proteases that are active on Soy
Amino acid “A” at position 576 in the alignment.
Amino acid “C” at position 460 in the alignment.
Amino acid “L” at position 404 in the alignment.
Amino acid “A” at position 437 in the alignment.
Amino acid “A” at position 438 in the alignment.
Amino acid “H” at position 441 in the alignment.
Amino acid “F” at position 380 in the alignment.
   Active site amino acids that are unique to proteases that are active on Hemp protein powder:
Amino acid “L” at position 404 in the alignment.
Amino acid “E” at position 441 in the alignment.
   Active site amino acids that are unique to proteases that are active on Broccoli:
Amino acid “H” at position 441 in the alignment.
Amino acid “L” at position 404 in the alignment.
Amino acid “C” at position 460 in the alignment.
Amino acid “A” at position 438 in the alignment.
   Active site amino acids that are unique to proteases that are active on Buckwheat:
Amino acid “H” at position 441 in the alignment.
Amino acid “L” at position 404 in the alignment.
Amino acid “C” at position 460 in the alignment.
   Active site amino acids that are unique to proteases that are active on Chia seeds:
Amino acid “H” at position 441 in the alignment.
Amino acid “L” at position 404 in the alignment.
Amino acid “N” at position 460 in the alignment.
   Active site amino acids that are unique to proteases that are active on Kamut:
Amino acid “H” at position 441 in the alignment.
Amino acid “L” at position 404 in the alignment.
Amino acid “C” at position 460 in the alignment.
Amino acid “A” at position 438 in the alignment.
   Active site amino acids that are unique to proteases that are active on Rye berries:
Amino acid “E” at position 441 in the alignment.
Amino acid “L” at position 404 in the alignment.
   Active site amino acids that are unique to proteases that are active on Amaranth:
Amino acid “L” at position 404 in the alignment.
Amino acid “E” at position 441 in the alignment.
   Active site amino acids that are unique to proteases that are active on Barley:
Amino acid “H” at position 441 in the alignment.
Amino acid “L” at position 404 in the alignment.
Amino acid “C” at position 460 in the alignment.
   Active site amino acids that are unique to proteases that are active on Chicken Egg:
Amino acid “L” at position 404 in the alignment.
Amino acid “E” at position 441 in the alignment.
   Active site amino acids that are unique to proteases that are active on Spirulina:
Amino acid “H” at position 441 in the alignment.
Amino acid “L” at position 404 in the alignment.
Amino acid “C” at position 460 in the alignment.
   Active site amino acids that are unique to proteases that are active on Chlorella:
Amino acid “L” at position 404 in the alignment.
Amino acid “E” at position 441 in the alignment.
   Active site amino acids that are unique to proteases that are active on Peanut:
Amino acid “L” at position 404 in the alignment.
Amino acid “E” at position 441 in the alignment.
   Active site amino acids that are unique to proteases that are active on Almonds:
Amino acid “L” at position 404 in the alignment.
Amino acid “E” at position 441 in the alignment.
   Active site amino acids that are unique to proteases that are active on Cashews:
Amino acid “L” at position 404 in the alignment.
Amino acid “E” at position 441 in the alignment.
   Active site amino acids that are unique to proteases that are active on Pistachios:
Amino acid “L” at position 404 in the alignment.
Amino acid “E” at position 441 in the alignment.
   Active site amino acids that are unique to proteases that are active on Royal Canin:
Amino acid “E” at position 441 in the alignment.
Amino acid “L” at position 404 in the alignment.

### Food Supplements and Food Products

Proteases of the disclosure can be used in the manufacture of food supplements (e.g., dietary supplements, nutritional supplements, sports nutrition supplements, digestive aid supplements, and the like) of various dosage forms, including for example, tablet, capsule, powder, granule, pellet, soft gel, hard gel, controlled release form, liquid, syrup, suspension, emulsion, and the like. Any commercially acceptable formulation known to be suitable for use in food products may be used in the food supplements of the present disclosure. Thus, the food supplement of the disclosure may further comprise components such as a bulking agent, a carrier, a sweetener, a coating, a preservative, a binding agent, a desiccant, a lubricating agent, a filler, a solubilizing agent, an emulsifier, a stabilizer, a matrix modifier, and the like.

Examples of bulking agents suitable for use in the present disclosure include gum acacia, gum arabic, xanthan gum, guar gum, and pectin. Example of carriers include maltodextrin, polypropylene, starch, modified starch, gum, proteins, and amino acids. Examples of sweeteners include glucose, fructose, stevia, acesulfame potassium, and erythritol. Examples of coatings include ethyl cellulose, hydroxypropyl methyl cellulose, and shellac. Examples of preservatives include benzoic acid, benzyl alcohol, and calcium acetate. Examples of binding agents include croscarmellose sodium, povidone, and dextrin. Examples of desiccants include silicon dioxide, and calcium silicate. Examples of lubricating agents include magnesium stearate, stearic acid, and silicon dioxide. Examples of fillers include maltodextrin, dextrin, starch, and calcium salts. Examples of solubilizing agents include cyclodextrin, and lecithin. Examples of emulsifiers include vegetable oils, fatty acids and mono-, and di- and triglycerides, such as medium chain triglycerides or their esters. Suitable stabilizers include agar, pectin and lecithin. Suitable matrix modifiers are those with a buffering capacity between pH 1 and pH 6 and known to be suitable for use in food products. Examples include salts of weak organic and inorganic acids, such as flavonoids, flavonols, isoflavones, catechins, gallic acid, monohydrate or dihydrate phosphates, sulfates, ascorbates, amino acids, sodium citrate, citric acid, benzoates, gluconic acid, acetic acid, picolinic acid, nicotinic acid, and phenolic or polyphenolic compounds. One of ordinary skill in the art can readily determine the amount of each ingredient to be added to the food supplement.

As noted above, the present disclosure is based, at least in part, on the discovery of combinations of proteases, or combination of proteases, that are particularly effective in digesting certain target food proteins. The food supplement may be designed to be ingested with the food product comprising the target food protein or may be ingested just before or just after the food product, typically within 2 hours before or after ingesting the food product. Thus, for the purposes of the present disclosure, a protease of the disclosure, or a food supplement comprising the protease, is “ingested with” a food product, if it is ingested simultaneously with the food product or within 2 hours before or after ingestion of the food product. In those cases in which the protease is ingested simultaneously with the food product, the food supplement may not be a separate composition from the food product and the proteases and other food supplement components, if present, will be incorporated into the food product.

The food products used with the food supplements of the disclosure may be any food product comprising the food proteins identified here. Thus, for example, the food product may be an unprocessed plant or animal part (e.g., beans, peas, chicken parts, beef and the like) or may be a processed food product comprising or derived from one or more of the food proteins identified here. For example, the food products may comprise a plant or animal protein isolate or protein concentrate (e.g., soy protein, casein, or whey).

In the typical embodiment, a unit dose of a food supplement of the disclosure will typically comprise from about 0.01 mg/gram food protein or 0.001% (w/w) to about 50 mg/gram food protein or 5% (w/w), usually from about 1 mg/gram food protein or 0.1% (w/w) to 10 mg/gram food protein or 1.0% (w/w), of each protease.

One of skill will appreciate that the compositions of the disclosure, either food supplements or food products, can comprise more than one of the proteases of the disclosure. For example, the compositions may comprise one, two three, four, or more proteases that are effective for a single food product or group of food products.

### EXAMPLES

The following examples are offered to illustrate, but not to limit the claimed disclosure.

To fully realize the protein nutritional values in food, 12 proteolytic enzymes that were predicted to be active under acidic environment (pH 2.0-5.0) have been identified and characterized. These 12 proteases cover a diverse sequence space and multiple sequence alignment analysis reveals that they share an average pairwise sequence identity of 35% o. These enzymes have been recombinantly produced in E. coli and their proteolytic activities have been tested on a total of 57 food substrates. (Table 1)

TABLE 1
List of 57 food sources tested.

| | | | | | | |
|---|---|---|---|---|---|---|
| Mung | Field | Yellow | Pork | Pea | Rye | Cashews |
| beans | Peas | split peas | | Protein | berries | |
| | | | | powder | | |
| Green | Cowpea | Navy pea | Chicken | | Amaranth | Pistachios |
| beans | | | | | | |
| Kidney | Baby | Black | Turkey | Soy | Barley | Royal |
| beans | Lima | eyed peas | | | | Canin |
| Pea | Crowder | Masdoor | Beef | Hemp | Chicken | |
| | pea | Dal | | protein | Egg | |
| | | (Indian | | powder | | |
| | | Red | | | | |
| | | lentils) | | | | |
| Pinto | Pink | Great | Flounder | Broccoli | Spirulina | |
| beans | beans | Northern | | | | |
| | | Beans | | | | |
| Black | Adzuki | Cranberry | Yogurt | Quinoa | Chlorella | |
| beans | beans | beans | | | | |
| Lentil | Lady | White | Asparagus | Buckwheat | Peanut | |
| | cream | beans | | | | |
| | peas | | | | | |
| Chickpea | Cannellini | Fava | Whey | Chia seeds | Sunflower | |
| | beans | beans | | | seeds | |
| Lupine | Pigeon | Salmon | Casein | Kamut | Almonds | |
| Beans | Peas | | | | | |

The digestive properties of each enzyme were examined using SDS-PAGE electrophoretic analysis and a wide range of proteolytic activities were found. Proteolytic activity of each enzyme was determined as follows. The protease activity is measured using sodium dodecyl sulfate-polyacrylamide gel electrophoresis (SDS-PAGE). The digestion assay for each food-protease pair was performed by incubating 2 μM of each individual protease with each food source (Table 2) at 37° C. for 12 hours at pH 4.5 in reaction buffer (100 mM acetate 100 mM NaCl). The samples were subsequently spun down at 4,700 rpm for 10 minutes and heated at 70° C. for 10 minutes in 1× Laemmli buffer. The samples were then loaded onto a 12% polyacrylamide gel for proteolytic products separation and the gel was stained with commassie blue stains for protein bands visualization. Protease activities were determined by monitoring the disappearance of protein bands compared to a negative control sample where no protease was added to the reaction mixture.

TABLE 2
Amount of food protein used in each proteolytic digest reaction.

| | Milligrams of food in |
|---|---|
| Protein Source | 1 ml of reaction buffer |
| Adzuki beans | 200.00 |
| Almonds | 30 |
| Amaranth | 400.00 |
| Asparagus | 600.00 |
| Baby Lima | 200.00 |
| Barley | 800 |
| Beef | 66.00 |
| Black beans | 195.00 |
| Blackeyed peas | 200.00 |
| Broccoli | 528.00 |
| Buckwheat | 672.00 |
| Cannellini beans | 200.00 |
| Casein | 10.00 |
| Cashews | 30 |
| Chia seeds | 30.00 |
| Chicken | 66.00 |
| Chicken Egg | 126.00 |
| Chickpea | 108.00 |
| Chlorella | 15.00 |
| Cowpea | 200.00 |
| Cranberry beans | 200.00 |
| Crowder pea | 200.00 |
| Fava beans | 200.00 |
| Field Peas | 200.00 |
| Flounder | 66.00 |
| Great Northern Beans | 200.00 |
| Green beans | 130.00 |
| Hemp protein powder | 5.00 |
| Kamut | 400.00 |
| Kidney beans | 470.00 |
| Lady cream peas | 200.00 |
| Lentil | 164.00 |
| Lupine beans | 195.00 |
| Masdoor Dal | 400.00 |

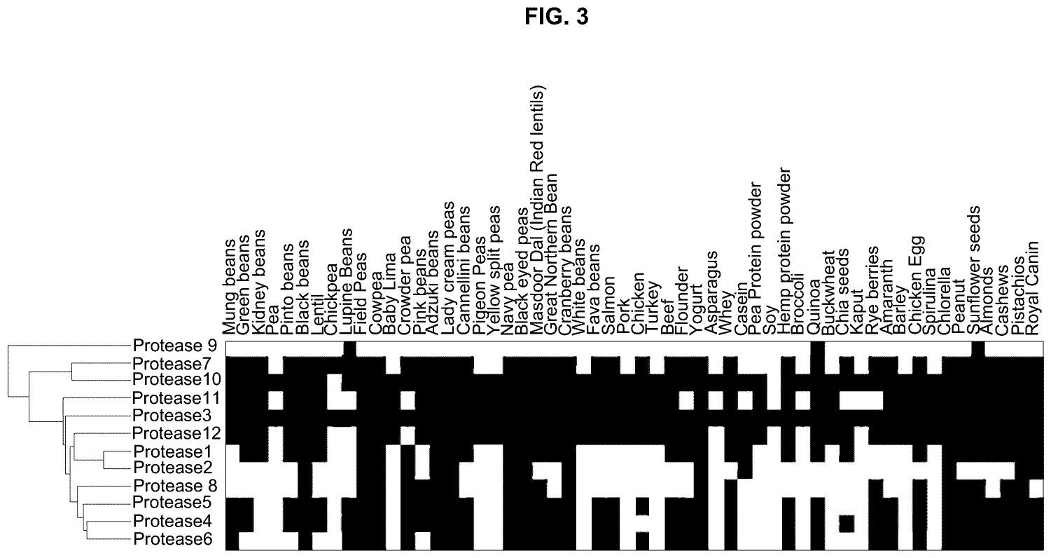

Results showed that these proteolytic enzymes, when added to the food sources tested, degraded the major protein species into smaller peptides with diverse activities and specificities (FIG. 3). Each of these proteases provide unique functions that allow the targeted digestion of the major protein species in each individual food source tested.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### REFERENCES

1. Moughan, P. J., Amino acid availability: aspects of chemical analysis and bioassay methodology. Nutrition Research Reviews 2003, 16 (2), 127-141.
2. Elango, R.; Levesque, C.; Ball, R. O.; Pencharz, P. B., Available versus digestible amino acids-new stable isotope methods. British Journal of Nutrition 2012, 108 (S2), 5306-5314.
3. Mišurcová L., Seaweed digestibility and methods used for digestibility determination. Handbook of Marine Macroalgae: Biotechnology and Applied Phycology 2011, 285-301.
4. Lee, W. T.; Weisell, R.; Albert, J.; Tomé, D.; Kurpad, A. V.; Uauy, R., Research Approaches and Methods for Evaluating the Protein Quality of Human Foods Proposed by an FAO Expert Working Group in 2014-. The Journal of nutrition 2016, 146 (5), 929-932.
5. Millward, D. J.; Layman, D. K.; Tomé, D.; Schaafsma, G., Protein quality assessment: impact of expanding understanding of protein and amino acid needs for optimal health-. The Americanjournal of clinical nutrition 2008, 87 (5), 1576S-1581S.
6. Matthews, D. M.; Adibi, S. A., Peptide absorption. Gastroenterology 1976, 71 (1), 151-161.
7. Sarwar, G.; Peace, R. W.; Botting, H. G.; Brulé, D., Digestibility of protein and amino acids in selected foods as determined by a rat balance method. Plant Foods for Human Nutrition 1989, 39 (1), 23-32.
8. Savoie, L.; Charbonneau, R.; Parent, G., In vitro amino acid digestibility of food proteins as measured by the digestion cell technique. Plant Foods for Human Nutrition 1989, 39 (1), 93-107.
9. Mandalari, G.; Adel-Patient, K.; Barkholt, V.; Baro, C.; Bennett, L.; Bublin, M.; Gaier, S.; Graser, G.; Ladics, G.; Mierzejewska, D., In vitro digestibility of β-casein and β-lactoglobulin under simulated human gastric and duodenal conditions: a multi-laboratory evaluation. Regulatory Toxicology and Pharmacology 2009, 55 (3), 372-381.
10. Pennings, B.; Boirie, Y.; Senden, J. M.; Gijsen, A. P.; Kuipers, H.; van Loon, L. J., Whey protein stimulates postprandial muscle protein accretion more effectively than do casein and casein hydrolysate in older men-. The American journal of clinical nutrition 2011, 93 (5), 997-1005.
11. Koopman, R.; Crombach, N.; Gijsen, A. P.; Walrand, S.; Fauquant, J.; Kies, A. K.; Lemosquet, S.; Saris, W. H.; Boirie, Y.; van Loon, L. J., Ingestion of a protein hydrolysate is accompanied by an accelerated in vivo digestion and absorption rate when compared with its intact protein-. The American journal of clinical nutrition 2009, 90 (1), 106-115.
12. Oben, J.; Kothari, S. C.; Anderson, M. L., An open label study to determine the effects of an oral proteolytic enzyme system on whey protein concentrate metabolism in healthy males. Journal of the International Society of Sports Nutrition 2008, 5 (1), 10.
13. Astwood, James D., John N. Leach, and Roy L. Fuchs. “Stability of food allergens to digestion in vitro.” Nature biotechnology 14.10 (1996): 1269.
14. Takagi, Kayoko, et al. “Comparative study of in vitro digestibility of food proteins and effect of preheating on the digestion.” Biological and Pharmaceutical Bulletin 26.7 (2003): 969-973.
15. Fu, Tong-Jen, Upasana R. Abbott, and Catherine Hatzos. “Digestibility of food allergens and nonallergenic proteins in simulated gastric fluid and simulated intestinal fluid a comparative study.” Journal of agricultural and food chemistry 50.24 (2002): 7154-7160.

1. A method of improving digestion of proteins in a food product by a subject, the method comprising ingesting with the food product, a food supplement which comprises one or more proteases having an amino acid sequence at least substantially identical to an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, and SEQ ID NO: 24.

2. The method of claim 1, wherein the food product comprises:
a) a legume source protein and the food supplement which comprises one or more proteases having at least substantially identical to an amino acid sequence selected from the group consisting of SEQ ID NO: 2, SEQ ID NO: 4, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, and SEQ ID NO: 24; or
b) a non-legume plant source protein and the food supplement which comprises one or more proteases having at least substantially identical to an amino acid sequence selected from the group consisting of SEQ ID NO: 2, SEQ ID NO: 4, SEQ ID NO: 8, SEQ ID NO: 10, SEQ ID NO: 12, SEQ ID NO: 14, SEQ ID NO: 16, SEQ ID NO: 18, SEQ ID NO: 22, and SEQ ID NO: 24; or
c) an animal source protein and the food supplement which comprises one or more proteases having at least substantially identical to an amino acid sequence selected from the group consisting of SEQ ID NO: 2, SEQ ID NO: 4, SEQ ID NO: 8, SEQ ID NO: 10, SEQ ID NO: 12, SEQ ID NO: 14, SEQ ID NO: 16, SEQ ID NO: 18, SEQ ID NO: 22, and SEQ ID NO: 24.

3. The method of claim 1, wherein the food product comprises:
a) mung bean protein and the food supplement which comprises one or more proteases having an amino acid sequence at least substantially identical to an amino acid sequence selected from the group consisting of SEQ ID NO: 18, SEQ ID NO: 2, SEQ ID NO: 16, and SEQ ID NO: 4; or
b) green bean protein and the food supplement which comprises one or more proteases having an amino acid sequence at least substantially identical to an amino acid sequence selected from the group consisting of SEQ ID NO: 18, SEQ ID NO: 12, SEQ ID NO: 16, and SEQ ID NO: 4; or
c) kidney bean protein and the food supplement which comprises one or more proteases having an amino acid sequence at least substantially identical to an amino acid sequence selected from the group consisting of SEQ ID NO: 18, SEQ ID NO: 12, SEQ ID NO: 8, SEQ ID NO: 16, SEQ ID NO: 4, and SEQ ID NO: 10; or
d) pea, broccoli, kamut, or asparagus protein and the food supplement which comprises one or more proteases having an amino acid sequence selected at least substantially identical to an amino acid sequence from the group consisting of SEQ ID NO: 18, SEQ ID NO: 12, SEQ ID NO: 22, SEQ ID NO: 14, SEQ ID NO: 8, SEQ ID NO: 2, SEQ ID NO: 16, SEQ ID NO: 24, SEQ ID NO: 4, and SEQ ID NO: 10; or
e) pinto bean and lentil bean protein and the food supplement which comprises one or more proteases having an amino acid sequence at least substantially identical to an amino acid sequence selected from the group consisting of SEQ ID NO: 18, SEQ ID NO: 12, SEQ ID NO: 16, and SEQ ID NO: 4; or
f) black bean, field pea, cow pea, adzuki bean, lady cream pea, navy pea, black-eyed pea, cranberry bean, yogurt, chlorella, or pistachio protein and the food supplement which comprises a protease having an amino acid sequence at least substantially identical to the amino acid sequence of SEQ ID NO: 18; or
g) chick pea protein and the food supplement which comprises one or more proteases having an amino acid sequence selected at least substantially identical to an amino acid sequence from the group consisting of SEQ ID NO: 18, SEQ ID NO: 12, SEQ ID NO: 22, SEQ ID NO: 8, SEQ ID NO: 2, SEQ ID NO: 16, SEQ ID NO: 24, SEQ ID NO: 4, SEQ ID NO: 20, and SEQ ID NO: 10; or
h) lupine bean protein and the food supplement which comprises one or more proteases having an amino acid sequence selected at least substantially identical to an amino acid sequence from the group consisting of SEQ ID NO: 22, SEQ ID NO: 2, SEQ ID NO: 16, SEQ ID NO: 24, and SEQ ID NO: 4; or
i) baby lima bean protein and the food supplement which comprises one or more proteases having an amino acid sequence selected at least substantially identical to an amino acid sequence from the group consisting of SEQ ID NO: 18, SEQ ID NO: 12, SEQ ID NO: 14, SEQ ID NO: 8, SEQ ID NO: 2, SEQ ID NO: 16, SEQ ID NO: 4, and SEQ ID NO: 10; or
j) crowder pea protein and the food supplement which comprises one or more proteases having an amino acid sequence selected at least substantially identical to an amino acid sequence from the group consisting of SEQ ID NO: 18, SEQ ID NO: 22, and SEQ ID NO: 24; or
k) pink bean protein and the food supplement which comprises one or more proteases having an amino acid sequence selected at least substantially identical to an amino acid sequence from the group consisting of SEQ ID NO: 18, SEQ ID NO: 12, SEQ ID NO: 2, and SEQ ID NO: 4; or
l) cannellini bean protein and the food supplement which comprises one or more proteases having an amino acid sequence selected at least substantially identical to an amino acid sequence from the group consisting of SEQ ID NO: 18, SEQ ID NO: 16, and SEQ ID NO: 4; or
m) pigeon pea, yellow split pea, white bean, pork, pea protein powder, buckwheat, barley, or turkey protein and the food supplement which comprises one or more proteases having an amino acid sequence selected at least substantially identical to an amino acid sequence from the group consisting of SEQ ID NO: 18, SEQ ID NO: 12, SEQ ID NO: 14, SEQ ID NO: 8, SEQ ID NO: 2, SEQ ID NO: 16, SEQ ID NO: 4, and SEQ ID NO: 10; or
n) Indian red lentil bean, whey, peanut, cashew, or chicken egg protein and the food supplement which comprises one or more proteases having an amino acid sequence at least substantially identical to an amino acid sequence selected from the group consisting of SEQ ID NO: 18, and SEQ ID NO: 4; or
o) great northern bean, hemp protein powder, almond, or beef protein and the food supplement which comprises one or more proteases having an amino acid sequence at least substantially identical to an amino acid sequence selected from the group consisting of SEQ ID NO: 18, SEQ ID NO: 16, and SEQ ID NO: 4; or
p) fava bean or salmon protein and the food supplement which comprises one or more proteases having an amino acid sequence at least substantially identical to an amino acid sequence selected from the group consisting of SEQ ID NO: 18, SEQ ID NO: 2, SEQ ID NO: 16, and SEQ ID NO: 4; or
q) chicken protein and the food supplement which comprises one or more proteases having an amino acid sequence at least substantially identical to an amino acid sequence selected from the group consisting of SEQ ID NO: 18, SEQ ID NO: 8, SEQ ID NO: 2, SEQ ID NO: 16, and SEQ ID NO: 4; or
r) flounder protein and the food supplement which comprises one or more proteases having an amino acid sequence at least substantially identical to an amino acid sequence selected from the group consisting of SEQ ID NO: 18, SEQ ID NO: 22, SEQ ID NO: 16, and SEQ ID NO: 4; or
s) casein and the food supplement which comprises one or more proteases having an amino acid sequence selected at least substantially identical to an amino acid sequence from the group consisting of SEQ ID NO: 18, SEQ ID NO: 12, SEQ ID NO: 22, SEQ ID NO: 14, SEQ ID NO: 8, SEQ ID NO: 16, and SEQ ID NO: 10; or
t) quinoa protein and the food supplement which comprises one or more proteases having an amino acid sequence at least substantially identical to an amino acid sequence selected from the group consisting of SEQ ID NO: 16, and SEQ ID NO: 4; or
u) chia seed protein and the food supplement which comprises one or more proteases having an amino acid sequence selected at least substantially identical to an amino acid sequence from the group consisting of SEQ ID NO: 18, SEQ ID NO: 12, SEQ ID NO: 22, SEQ ID NO: 16, SEQ ID NO: 4, and SEQ ID NO: 10; or
v) soy bean protein and the food supplement which comprises one or more proteases having an amino acid sequence at least substantially identical to an amino acid sequence selected from the group consisting of SEQ ID NO: 18, SEQ ID NO: 12, SEQ ID NO: 22, SEQ ID NO: 14, SEQ ID NO: 8, SEQ ID NO: 2, SEQ ID NO: 16, SEQ ID NO: 24, SEQ ID NO: 4, SEQ ID NO: 20, and SEQ ID NO: 10; or
w) rye berry protein and the food supplement which comprises one or more proteases having an amino acid sequence at least substantially identical to an amino acid sequence selected from the group consisting of SEQ ID NO: 18, SEQ ID NO: 22, SEQ ID NO: 2, and SEQ ID NO: 4; or
x) amaranth protein and the food supplement which comprises one or more proteases having an amino acid sequence at least substantially identical to an amino acid sequence selected from the group consisting of SEQ ID NO: 18, SEQ ID NO: 2, SEQ ID NO: 16, and SEQ ID NO: 4; or
y) spirulina protein and the food supplement which comprises one or more proteases having an amino acid sequence at least substantially identical to an amino acid sequence selected from the group consisting of SEQ ID NO: 18, SEQ ID NO: 12, SEQ ID NO: 14, SEQ ID NO: 8, SEQ ID NO: 2, SEQ ID NO: 16, SEQ ID NO: 4, and SEQ ID NO: 10; or
z) sunflower seed protein and the food supplement which comprises a protease having an amino acid sequence at least substantially identical to the amino acid sequence of SEQ ID NO: 4.

4. A method of improving digestion of proteins in a food product by a subject, the method comprising ingesting with the food product, a food supplement which comprises one or more proteases comprising an active site sequence at least substantially identical to the active site sequence in a protease having an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, and SEQ ID NO: 24.

5. The method of claim 4, wherein the food product comprises:
a) a legume source protein and the food supplement which comprises one or more proteases comprising an active site sequence at least substantially identical to the active site sequence in a protease having an amino acid sequence selected from the group consisting of SEQ ID NO: 2, SEQ ID NO: 4, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, and SEQ ID NO: 24; or
b) a non-legume plant source protein and the food supplement which comprises one or more proteases comprising an active site sequence at least substantially identical to the active site sequence in a protease having an amino acid sequence selected from the group consisting of SEQ ID NO: 2, SEQ ID NO: 4, SEQ ID NO: 8, SEQ ID NO: 10, SEQ ID NO: 12, SEQ ID NO: 14, SEQ ID NO: 16, SEQ ID NO: 18, SEQ ID NO: 22, and SEQ ID NO: 24; or
c) an animal source protein and the food supplement which comprises one or more proteases comprising an active site sequence at least substantially identical to the active site sequence in a protease having an amino acid sequence selected from the group consisting of SEQ ID NO: 2, SEQ ID NO: 4, SEQ ID NO: 8, SEQ ID NO: 10, SEQ ID NO: 12, SEQ ID NO: 14, SEQ ID NO: 16, SEQ ID NO: 18, SEQ ID NO: 22, and SEQ ID NO: 24.

6. The method of claim 4, wherein the food product comprises:
a) mung bean protein and the food supplement which comprises one or more proteases comprising an active site sequence at least substantially identical to the active site sequence in a protease having an amino acid sequence selected from the group consisting of SEQ ID NO: 18, SEQ ID NO: 2, SEQ ID NO: 16, and SEQ ID NO: 4; or
b) green bean protein and the food supplement which comprises one or more proteases comprising an active site sequence at least substantially identical to the active site sequence in a protease having an amino acid sequence selected from the group consisting of SEQ ID NO: 18, SEQ ID NO: 12, SEQ ID NO: 16, and SEQ ID NO: 4; or
c) kidney bean protein and the food supplement which comprises one or more proteases comprising an active site sequence at least substantially identical to the active site sequence in a protease having an amino acid sequence selected from the group consisting of SEQ ID NO: 18, SEQ ID NO: 12, SEQ ID NO: 8, SEQ ID NO: 16, SEQ ID NO: 4, and SEQ ID NO: 10; or
d) pea, broccoli, kamut, or asparagus protein and the food supplement which comprises one or more proteases comprising an active site sequence at least substantially identical to the active site sequence in a protease having an amino acid sequence selected from the group consisting of SEQ ID NO: 18, SEQ ID NO: 12, SEQ ID NO: 22, SEQ ID NO: 14, SEQ ID NO: 8, SEQ ID NO: 2, SEQ ID NO: 16, SEQ ID NO: 24, SEQ ID NO: 4, and SEQ ID NO: 10; or
e) pinto bean and lentil bean protein and the food supplement which comprises one or more proteases comprising an active site sequence at least substantially identical to the active site sequence in a protease having an amino acid sequence selected from the group consisting of SEQ ID NO: 18, SEQ ID NO: 12, SEQ ID NO: 16, and SEQ ID NO: 4; or
f) black bean, field pea, cow pea, adzuki bean, lady cream pea, navy pea, black-eyed pea, cranberry bean, yogurt, chlorella, or pistachio protein and the food supplement which comprises one or more proteases comprising an active site sequence at least substantially identical to the active site sequence in a protease having the amino acid sequence of SEQ ID NO: 18; or
g) chick pea protein and the food supplement which comprises one or more proteases comprising an active site sequence at least substantially identical to the active site sequence in a protease having an amino acid sequence selected from the group consisting of SEQ ID NO: 18, SEQ ID NO: 12, SEQ ID NO: 22, SEQ ID NO: 8, SEQ ID NO: 2, SEQ ID NO: 16, SEQ ID NO: 24, SEQ ID NO: 4, SEQ ID NO: 20, and SEQ ID NO: 10; or
h) lupine bean protein and the food supplement which comprises one or more proteases comprising an active site sequence at least substantially identical to the active site sequence in a protease having an amino acid sequence selected from the group consisting of SEQ ID NO: 22, SEQ ID NO: 2, SEQ ID NO: 16, SEQ ID NO: 24, and SEQ ID NO: 4; or
i) baby lima bean protein and the food supplement which comprises one or more proteases comprising an active site sequence at least substantially identical to the active site sequence in a protease having an amino acid sequence selected from the group consisting of SEQ ID NO: 18, SEQ ID NO: 12, SEQ ID NO: 14, SEQ ID NO: 8, SEQ ID NO: 2, SEQ ID NO: 16, SEQ ID NO: 4, and SEQ ID NO: 10; or
j) crowder pea protein and the food supplement which comprises one or more proteases comprising an active site sequence at least substantially identical to the active site sequence in a protease having an amino acid sequence selected from the group consisting of SEQ ID NO: 18, SEQ ID NO: 22, and SEQ ID NO: 24; or
k) pink bean protein and the food supplement which comprises one or more proteases comprising an active site sequence at least substantially identical to the active site sequence in a protease having an amino acid sequence selected from the group consisting of SEQ ID NO: 18, SEQ ID NO: 12, SEQ ID NO: 2, and SEQ ID NO: 4; or
l) cannellini bean protein and the food supplement which comprises one or more proteases comprising an active site sequence at least substantially identical to the active site sequence in a protease having an amino acid sequence selected from the group consisting of SEQ ID NO: 18, SEQ ID NO: 16, and SEQ ID NO: 4; or
m) pigeon pea, yellow split pea, white bean, pork, pea protein powder, buckwheat, barley, or turkey protein and the food supplement which comprises one or more proteases comprising an active site sequence at least substantially identical to the active site sequence in a protease having an amino acid sequence selected from the group consisting of SEQ ID NO: 18, SEQ ID NO: 12, SEQ ID NO: 14, SEQ ID NO: 8, SEQ ID NO: 2, SEQ ID NO: 16, SEQ ID NO: 4, and SEQ ID NO: 10; or
n) Indian red lentil bean, whey, peanut, cashew, or chicken egg protein and the food supplement which comprises one or more proteases comprising an active site sequence at least substantially identical to the active site sequence in a protease having an amino acid sequence selected from the group consisting of SEQ ID NO: 18, and SEQ ID NO: 4; or
o) great northern bean, hemp protein powder, almond, or beef protein and the food supplement which comprises one or more proteases comprising an active site sequence at least substantially identical to the active site sequence in a protease having an amino acid sequence selected from the group consisting of SEQ ID NO: 18, SEQ ID NO: 16, and SEQ ID NO: 4; or
p) fava bean or salmon protein and the food supplement which comprises one or more proteases comprising an active site sequence at least substantially identical to the active site sequence in a protease having an amino acid sequence selected from the group consisting of SEQ ID NO: 18, SEQ ID NO: 2, SEQ ID NO: 16, and SEQ ID NO: 4; or
q) chicken protein and the food supplement which comprises one or more proteases comprising an active site sequence at least substantially identical to the active site sequence in a protease having an amino acid sequence selected from the group consisting of SEQ ID NO: 18, SEQ ID NO: 8, SEQ ID NO: 2, SEQ ID NO: 16, and SEQ ID NO: 4; or
r) flounder protein and the food supplement which comprises one or more proteases comprising an active site sequence at least substantially identical to the active site sequence in a protease having an amino acid sequence selected from the group consisting of SEQ ID NO: 18, SEQ ID NO: 22, SEQ ID NO: 16, and SEQ ID NO: 4; or
s) casein and the food supplement which comprises one or more proteases comprising an active site sequence at least substantially identical to the active site sequence in a protease having an amino acid sequence selected from the group consisting of SEQ ID NO: 18, SEQ ID NO: 12, SEQ ID NO: 22, SEQ ID NO: 14, SEQ ID NO: 8, SEQ ID NO: 16, and SEQ ID NO: 10; or
t) quinoa protein and the food supplement which comprises one or more proteases comprising an active site sequence at least substantially identical to the active site sequence in a protease having an amino acid sequence selected from the group consisting of SEQ ID NO: 16, and SEQ ID NO: 4; or
u) chia seed protein and the food supplement which comprises one or more proteases comprising an active site sequence at least substantially identical to the active site sequence in a protease having an amino acid sequence selected from the group consisting of SEQ ID NO: 18, SEQ ID NO: 12, SEQ ID NO: 22, SEQ ID NO: 16, SEQ ID NO: 4, and SEQ ID NO: 10; or
v) soy bean protein and the food supplement which comprises one or more proteases comprising an active site sequence at least substantially identical to the active site sequence in a protease having an amino acid sequence selected from the group consisting of SEQ ID NO: 18, SEQ ID NO: 12, SEQ ID NO: 22, SEQ ID NO: 14, SEQ ID NO: 8, SEQ ID NO: 2, SEQ ID NO: 16, SEQ ID NO: 24, SEQ ID NO: 4, SEQ ID NO: 20, and SEQ ID NO: 10; or
w) rye berry protein and the food supplement which comprises one or more proteases comprising an active site sequence at least substantially identical to the active site sequence in a protease having an amino acid sequence selected from the group consisting of SEQ ID NO: 18, SEQ ID NO: 22, SEQ ID NO: 2, and SEQ ID NO: 4; or
x) amaranth protein and the food supplement which comprises one or more proteases comprising an active site having an amino acid sequence at least substantially identical to the active site in a protease having an amino acid sequence selected from the group consisting of SEQ ID NO: 18, SEQ ID NO: 2, SEQ ID NO: 16, and SEQ ID NO: 4; or
y) spirulina protein and the food supplement which comprises one or more proteases comprising an active site sequence at least substantially identical to the active site sequence in a protease having an amino acid sequence selected from the group consisting of SEQ ID NO: 18, SEQ ID NO: 12, SEQ ID NO: 14, SEQ ID NO: 8, SEQ ID NO: 2, SEQ ID NO: 16, SEQ ID NO: 4, and SEQ ID NO: 10; or
z) sunflower seed protein and the food supplement which comprises a protease comprising an active site sequence at least substantially identical to the active site sequence in a protease having the amino acid sequence of SEQ ID NO: 4.

7. The method of claim 1, wherein the food supplement is ingested simultaneously with the food product.

8. The method of claim 7, wherein the food supplement is incorporated into the food product.

9. A food supplement or food product comprising one or more proteases having an amino acid sequence at least substantially identical to an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, and SEQ ID NO: 24.

10. A food supplement or food product comprising one or more proteases comprising an active site sequence at least substantially identical to the active site sequence in a protease having an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, and SEQ ID NO: 24.

11. The food supplement or food product of claim 9, further comprising a one or more of a bulking agent, a carrier, a sweetener, a coating, a preservative, a binding agent, a desiccant, a lubricating agent, a filler, a solubilizing agent, an emulsifier, a stabilizer, or a matrix modifier.

12. The food supplement of claim 9, which is in a tablet, capsule, powder, granule, pellet, soft gel, hard gel, controlled release form, liquid, syrup, suspension, or emulsion form.

13. The food supplement or food product of claim 9 comprising
a) one or more proteases having at least substantially identical to an amino acid sequence selected from the group consisting of SEQ ID NO: 2, SEQ ID NO: 4, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, and SEQ ID NO: 24 and optionally comprises a legume source protein; or
b) one or more proteases having at least substantially identical to an amino acid sequence selected from the group consisting of SEQ ID NO: 2, SEQ ID NO: 4, SEQ ID NO: 8, SEQ ID NO: 10, SEQ ID NO: 12, SEQ ID NO: 14, SEQ ID NO: 16, SEQ ID NO: 18, SEQ ID NO: 22, and SEQ ID NO: 24 and optionally comprises a non-legume plant source protein; or
c) one or more proteases having at least substantially identical to an amino acid sequence selected from the group consisting of SEQ ID NO: 2, SEQ ID NO: 4, SEQ ID NO: 8, SEQ ID NO: 10, SEQ ID NO: 12, SEQ ID NO: 14, SEQ ID NO: 16, SEQ ID NO: 18, SEQ ID NO: 22, and SEQ ID NO: 24 and optionally comprises an animal source protein.

14. The food supplement or food product of claim 9 comprising
a) one or more proteases having an amino acid sequence at least substantially identical to an amino acid sequence selected from the group consisting of SEQ ID NO: 18, SEQ ID NO: 2, SEQ ID NO: 16, and SEQ ID NO: 4 and optionally comprises mung bean protein; or
b) one or more proteases having an amino acid sequence at least substantially identical to an amino acid sequence selected from the group consisting of SEQ ID NO: 18, SEQ ID NO: 12, SEQ ID NO: 16, and SEQ ID NO: 4 and optionally comprises green bean protein; or
c) one or more proteases having an amino acid sequence at least substantially identical to an amino acid sequence selected from the group consisting of SEQ ID NO: 18, SEQ ID NO: 12, SEQ ID NO: 8, SEQ ID NO: 16, SEQ ID NO: 4, and SEQ ID NO: 10 and optionally comprises kidney bean protein; or
d) one or more proteases having an amino acid sequence at least substantially identical to an amino acid sequence selected from the group consisting of SEQ ID NO: 18, SEQ ID NO: 12, SEQ ID NO: 22, SEQ ID NO: 14, SEQ ID NO: 8, SEQ ID NO: 2, SEQ ID NO: 16, SEQ ID NO: 24, SEQ ID NO: 4, and SEQ ID NO: 10 and optionally comprises pea, broccoli, kamut, or asparagus protein, or
e) one or more proteases having an amino acid sequence at least substantially identical to an amino acid sequence selected from the group consisting of SEQ ID NO: 18, SEQ ID NO: 12, SEQ ID NO: 16, and SEQ ID NO: 4 and optionally comprises pinto bean or lentil bean protein; or
f) a protease having an amino acid sequence at least substantially identical to the amino acid sequence of SEQ ID NO: 18 and optionally comprises black bean, field pea, cow pea, adzuki bean, lady cream pea, navy pea, black-eyed pea, cranberry bean, yogurt, chlorella, or pistachio protein; or
g) one or more proteases having an amino acid sequence at least substantially identical to an amino acid sequence selected from the group consisting of SEQ ID NO: 18, SEQ ID NO: 12, SEQ ID NO: 22, SEQ ID NO: 8, SEQ ID NO: 2, SEQ ID NO: 16, SEQ ID NO: 24, SEQ ID NO: 4, SEQ ID NO: 20, and SEQ ID NO: 10 and optionally comprises chick pea protein; or
h) one or more proteases having an amino acid sequence at least substantially identical to an amino acid sequence selected from the group consisting of SEQ ID NO: 22, SEQ ID NO: 2, SEQ ID NO: 16, SEQ ID NO: 24, and SEQ ID NO: 4 and optionally comprises lupine bean protein; or
i) one or more proteases having an amino acid sequence at least substantially identical to an amino acid sequence selected from the group consisting of SEQ ID NO: 18, SEQ ID NO: 12, SEQ ID NO: 14, SEQ ID NO: 8, SEQ ID NO: 2, SEQ ID NO: 16, SEQ ID NO: 4, and SEQ ID NO: 10 and optionally comprises baby lima bean protein; or
j) one or more proteases having an amino acid sequence at least substantially identical to an amino acid sequence selected from the group consisting of SEQ ID NO: 18, SEQ ID NO: 22, and SEQ ID NO: 24, and optionally comprises crowder pea protein; or
k) one or more proteases having an amino acid sequence at least substantially identical to an amino acid sequence selected from the group consisting of SEQ ID NO: 18, SEQ ID NO: 12, SEQ ID NO: 2, and SEQ ID NO: 4 and optionally comprises pink bean protein; or
l) one or more proteases having an amino acid sequence at least substantially identical to an amino acid sequence selected from the group consisting of SEQ ID NO: 18, SEQ ID NO: 16, and SEQ ID NO: 4 and optionally comprises cannellini bean protein; or
m) one or more proteases having an amino acid sequence at least substantially identical to an amino acid sequence selected from the group consisting of SEQ ID NO: 18, SEQ ID NO: 12, SEQ ID NO: 14, SEQ ID NO: 8, SEQ ID NO: 2, SEQ ID NO: 16, SEQ ID NO: 4, and SEQ ID NO: 10 and optionally comprises pigeon pea, yellow split pea, white bean, pork, pea protein powder, buckwheat, barley, or turkey protein; or
n) one or more proteases having an amino acid sequence at least substantially identical to an amino acid sequence selected from the group consisting of SEQ ID NO: 18, and SEQ ID NO: 4 and optionally comprises Indian red lentil bean, whey, peanut, cashew, or chicken egg protein; or
o) one or more proteases having an amino acid sequence at least substantially identical to an amino acid sequence selected from the group consisting of SEQ ID NO: 18, SEQ ID NO: 16, and SEQ ID NO: 4 and optionally comprises great northern bean, hemp protein powder, almond, or beef protein; or
p) one or more proteases having an amino acid sequence at least substantially identical to an amino acid sequence selected from the group consisting of SEQ ID NO: 18, SEQ ID NO: 2, SEQ ID NO: 16, and SEQ ID NO: 4 and optionally comprises fava bean or salmon protein; or
q) one or more proteases having an amino acid sequence at least substantially identical to an amino acid sequence selected from the group consisting of SEQ ID NO: 18, SEQ ID NO: 8, SEQ ID NO: 2, SEQ ID NO: 16, and SEQ ID NO: 4 and optionally comprises chicken protein; or
r) one or more proteases having an amino acid sequence at least substantially identical to an amino acid sequence selected from the group consisting of SEQ ID NO: 18, SEQ ID NO: 22, SEQ ID NO: 16, and SEQ ID NO: 4 and optionally comprises flounder protein; or
s) one or more proteases having an amino acid sequence at least substantially identical to an amino acid sequence selected from the group consisting of SEQ ID NO: 18, SEQ ID NO: 12, SEQ ID NO: 22, SEQ ID NO: 14, SEQ ID NO: 8, SEQ ID NO: 16, and SEQ ID NO: 10 and optionally comprises casein; or
t) one or more proteases having an amino acid sequence at least substantially identical to an amino acid sequence selected from the group consisting of SEQ ID NO: 16, and SEQ ID NO: 4 and optionally comprises quinoa protein; or
u) one or more proteases having an amino acid sequence at least substantially identical to an amino acid sequence selected from the group consisting of SEQ ID NO: 18, SEQ ID NO: 12, SEQ ID NO: 22, SEQ ID NO: 16, SEQ ID NO: 4, and SEQ ID NO: 10 and optionally comprises chia seed protein; or
v) one or more proteases having an amino acid sequence at least substantially identical to an amino acid sequence selected from the group consisting of SEQ ID NO: 18, SEQ ID NO: 12, SEQ ID NO: 22, SEQ ID NO: 14, SEQ ID NO: 8, SEQ ID NO: 2, SEQ ID NO: 16, SEQ ID NO: 24, SEQ ID NO: 4, SEQ ID NO: 20, and SEQ ID NO: 10 and optionally comprises soy bean protein; or
w) one or more proteases having an amino acid sequence at least substantially identical to an amino acid sequence selected from the group consisting of SEQ ID NO: 18, SEQ ID NO: 22, SEQ ID NO: 2, and SEQ ID NO: 4 and optionally comprises rye berry protein; or
x) one or more proteases having an amino acid sequence at least substantially identical to an amino acid sequence selected from the group consisting of SEQ ID NO: 18, SEQ ID NO: 2, SEQ ID NO: 16, and SEQ ID NO: 4 and optionally comprises amaranth protein; or
y) one or more proteases having an amino acid sequence at least substantially identical to an amino acid sequence selected from the group consisting of SEQ ID NO: 18, SEQ ID NO: 12, SEQ ID NO: 14, SEQ ID NO: 8, SEQ ID NO: 2, SEQ ID NO: 16, SEQ ID NO: 4, and SEQ ID NO: 10 and optionally comprises spirulina protein; or
z) a protease having an amino acid sequence at least substantially identical to the amino acid sequence of SEQ ID NO: 4 and optionally comprises sunflower seed protein.

15. The food supplement or food product of claim 9, comprising
a) one or more proteases comprising an active site sequence at least substantially identical to the active site sequence in a protease having an amino acid sequence selected from the group consisting of SEQ ID NO: 18, SEQ ID NO: 2, SEQ ID NO: 16, and SEQ ID NO: 4 and optionally comprises mung bean protein; or
b) one or more proteases comprising an active site sequence at least substantially identical to the active site sequence in a protease having an amino acid sequence selected from the group consisting of SEQ ID NO: 18, SEQ ID NO: 12, SEQ ID NO: 16, and SEQ ID NO: 4 and optionally comprises green bean protein; or
c) one or more proteases comprising an active site sequence at least substantially identical to the active site sequence in a protease having an amino acid sequence selected from the group consisting of SEQ ID NO: 18, SEQ ID NO: 12, SEQ ID NO: 8, SEQ ID NO: 16, SEQ ID NO: 4, and SEQ ID NO: 10 and optionally comprises kidney bean protein; or
d) one or more proteases comprising an active site sequence at least substantially identical to the active site sequence in a protease having an amino acid sequence selected from the group consisting of SEQ ID NO: 18, SEQ ID NO: 12, SEQ ID NO: 22, SEQ ID NO: 14, SEQ ID NO: 8, SEQ ID NO: 2, SEQ ID NO: 16, SEQ ID NO: 24, SEQ ID NO: 4, and SEQ ID NO: 10 and optionally comprises pea, broccoli, kamut, or asparagus protein; or
e) one or more proteases comprising an active site sequence at least substantially identical to the active site sequence in a protease having an amino acid sequence selected from the group consisting of SEQ ID NO: 18, SEQ ID NO: 12, SEQ ID NO: 16, and SEQ ID NO: 4 and optionally comprises pinto bean or lentil bean protein; or
f) a protease comprising an active site an active site sequence at least substantially identical to the active site sequence in a protease having the amino acid sequence of SEQ ID NO: 18 and optionally comprises black bean, field pea, cow pea, adzuki bean, lady cream pea, navy pea, black-eyed pea, cranberry bean, yogurt, chlorella, or pistachio protein; or
g) one or more proteases comprising an active site sequence at least substantially identical to the active site sequence in a protease having an amino acid sequence selected from the group consisting of SEQ ID NO: 18, SEQ ID NO: 12, SEQ ID NO: 22, SEQ ID NO: 8, SEQ ID NO: 2, SEQ ID NO: 16, SEQ ID NO: 24, SEQ ID NO: 4, SEQ ID NO: 20, and SEQ ID NO: 10 and optionally comprises chick pea protein; or
h) one or more proteases comprising an active site sequence at least substantially identical to the active site sequence in a protease having an amino acid sequence selected from the group consisting of SEQ ID NO: 22, SEQ ID NO: 2, SEQ ID NO: 16, SEQ ID NO: 24, and SEQ ID NO: 4 and optionally comprises lupine bean protein; or
i) one or more proteases having an active site sequence at least substantially identical to the active site sequence in a protease having an amino acid sequence selected from the group consisting of SEQ ID NO: 18, SEQ ID NO: 12, SEQ ID NO: 14, SEQ ID NO: 8, SEQ ID NO: 2, SEQ ID NO: 16, SEQ ID NO: 4, and SEQ ID NO: 10 and optionally comprises baby lima bean protein; or
j) one or more proteases comprising an active site sequence at least substantially identical to the active site sequence in a protease having an amino acid sequence selected from the group consisting of SEQ ID NO: 18, SEQ ID NO: 22, and SEQ ID NO: 24, and optionally comprises crowder pea protein; or
k) one or more proteases comprising an active site sequence at least substantially identical to the active site sequence in a protease having an amino acid sequence selected from the group consisting of SEQ ID NO: 18, SEQ ID NO: 12, SEQ ID NO: 2, and SEQ ID NO: 4 and optionally comprises pink bean protein; or
l) one or more proteases comprising an active site sequence at least substantially identical to the active site sequence in a protease having an amino acid sequence selected from the group consisting of SEQ ID NO: 18, SEQ ID NO: 16, and SEQ ID NO: 4 and optionally comprises cannellini bean protein; or
m) one or more proteases comprising an active site sequence at least substantially identical to the active site sequence in a protease having an amino acid sequence selected from the group consisting of SEQ ID NO: 18, SEQ ID NO: 12, SEQ ID NO: 14, SEQ ID NO: 8, SEQ ID NO: 2, SEQ ID NO: 16, SEQ ID NO: 4, and SEQ ID NO: 10 and optionally comprises pigeon pea, yellow split pea, white bean, pork, pea protein powder, buckwheat, barley, or turkey protein; or
n) one or more proteases comprising an active site sequence at least substantially identical to the active site sequence in a protease having an amino acid sequence selected from the group consisting of SEQ ID NO: 18, and SEQ ID NO: 4 and optionally comprises Indian red lentil bean, whey, peanut, cashew, or chicken egg protein; or
o) one or more proteases comprising an active site sequence at least substantially identical to the active site sequence in a protease having an amino acid sequence selected from the group consisting of SEQ ID NO: 18, SEQ ID NO: 16, and SEQ ID NO: 4 and optionally comprises great northern bean, hemp protein powder, almond, or beef protein; or
p) one or more proteases comprising an active site sequence at least substantially identical to the active site sequence in a protease having an amino acid sequence selected from the group consisting of SEQ ID NO: 18, SEQ ID NO: 2, SEQ ID NO: 16, and SEQ ID NO: 4 and optionally comprises fava bean or salmon protein; or
q) one or more proteases comprising an active site sequence at least substantially identical to the active site sequence in a protease having an amino acid sequence selected from the group consisting of SEQ ID NO: 18, SEQ ID NO: 8, SEQ ID NO: 2, SEQ ID NO: 16, and SEQ ID NO: 4 and optionally comprises chicken protein; or
r) one or more proteases comprising an active site sequence at least substantially identical to the active site sequence a protease having an amino acid sequence selected from the group consisting of SEQ ID NO: 18, SEQ ID NO: 22, SEQ ID NO: 16, and SEQ ID NO: 4 and optionally comprises flounder protein; or
s) one or more proteases comprising an active site sequence at least substantially identical to the active site sequence in a protease having an amino acid sequence selected from the group consisting of SEQ ID NO: 18, SEQ ID NO: 12, SEQ ID NO: 22, SEQ ID NO: 14, SEQ ID NO: 8, SEQ ID NO: 16, and SEQ ID NO: 10 and optionally comprises casein; or
t) one or more proteases comprising an active site sequence at least substantially identical to the active site sequence in a protease having an amino acid sequence selected from the group consisting of SEQ ID NO: 16, and SEQ ID NO: 4 and optionally comprises quinoa protein; or
u) one or more proteases an active site sequence at least substantially identical to the active site sequence in a protease having an amino acid sequence selected from the group consisting of SEQ ID NO: 18, SEQ ID NO: 12, SEQ ID NO: 22, SEQ ID NO: 16, SEQ ID NO: 4, and SEQ ID NO: 10 and optionally comprises chia seed protein; or
v) one or more proteases an active site sequence at least substantially identical to the active site sequence in a protease having an amino acid sequence selected from the group consisting of SEQ ID NO: 18, SEQ ID NO: 12, SEQ ID NO: 22, SEQ ID NO: 14, SEQ ID NO: 8, SEQ ID NO: 2, SEQ ID NO: 16, SEQ ID NO: 24, SEQ ID NO: 4, SEQ ID NO: 20, and SEQ ID NO: 10 and optionally comprises soy bean protein; or
w) one or more proteases comprising an active site sequence at least substantially identical to the active site sequence in a protease having an amino acid sequence selected from the group consisting of SEQ ID NO: 18, SEQ ID NO: 22, SEQ ID NO: 2, and SEQ ID NO: 4 and optionally comprises rye berry protein; or
x) one or more proteases comprising an active site sequence at least substantially identical to the active site sequence in a protease having an amino acid sequence selected from the group consisting of SEQ ID NO: 18, SEQ ID NO: 2, SEQ ID NO: 16, and SEQ ID NO: 4 and optionally comprises amaranth protein; or
y) one or more proteases comprising an active site sequence at least substantially identical to the active site sequence in a protease having an amino acid sequence selected from the group consisting of SEQ ID NO: 18, SEQ ID NO: 12, SEQ ID NO: 14, SEQ ID NO: 8, SEQ ID NO: 2, SEQ ID NO: 16, SEQ ID NO: 4, and SEQ ID NO: 10 and optionally comprises spirulina protein; or
z) a protease comprising an active an active site sequence at least substantially identical to the active site sequence in a protease having the amino acid sequence of SEQ ID NO: 4 and optionally comprises sunflower seed protein.

16. A method of making the food supplement of claim 9, the method comprising mixing one or more proteases having an amino acid sequence at least substantially identical to an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, and SEQ ID NO: 24, with one or more of a bulking agent, a carrier, a sweetener, a coating, a preservative, a binding agent, a desiccant, a lubricating agent, a filler, a solubilizing agent, an emulsifier, a stabilizer, or a matrix modifier.

17. The method of claim 16, wherein the proteases are recombinantly produced.

18. The method of claim 17, wherein the proteases are recombinantly produced in E. coli.

19. The method of claim 18, wherein the proteases are recombinantly produced using an expression cassette comprising a nucleic acid sequence at least substantially identical to an open reading from SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, and SEQ ID NO: 23.
